SEQUENCE LISTING (41 sequences)

SEQ ID NO: 1 - protein, 490 aa, synthetic construct
  VARIANT 11: note = X at position 11 is different from V; in an embodiment, is an amino acid residue with a hydrophobic side chain (A, I, L, M, F, Y or W); and in yet another embodiment, is a L.
  VARIANT 19: note = X at position 19 is any amino acid residue; in an embodiment, is different from M; in another embodiment, is an amino acid residue with a hydrophobic side chain (A, V, L, I, F, Y or W); and in yet another embodiment, is I.
  VARIANT 22: note = X at position 22 is any amino acid residue; in an embodiment is different from R; in another embodiment, is an amino acid residue with a positively-charged side chain (K or H); and in yet another embodiment, is K.
  VARIANT 33: note = X at position 33 is any amino acid residue; in an embodiment is different from G; in another embodiment, is an amino acid residue with a negatively-charged side chain (D or E); and in yet another embodiment, is D.
  VARIANT 41: note = X at position 41 is different from H; in an embodiment, is an amino acid residue with a polar uncharged side chain (S, T, N or Q); and in yet another embodiment, is Q
  VARIANT 87: note = X at position 87 is different from G; in an embodiment, is an amino acid residue with a negatively-charged side chain (D or E); and in yet another embodiment, is D.
  VARIANT 129: note = X at position 129 is different from F; in an embodiment, is an amino acid residue with a hydrophobic side chain (A, V, I, L, M, Y or W); and in yet another embodiment, is Y.
  VARIANT 136: note = X at position 136 is different from L; in an embodiment, is an amino acid residue with a hydrophobic side chain (A, V, I, M, F, Y or W); and in yet another embodiment, is F.
  VARIANT 173: note = X at position 173 is any amino acid residue; in an embodiment is different from R; in another embodiment, is an amino acid residue with a hydrophobic side chain (A, V, I, L, M, F, Y or W); and in yet another embodiment, is I.
  VARIANT 265: note = X at position 265 is different from R, in an embodiment, is an amino acid residue with a positively-charged side chain (K or H); and in yet another embodiment, is K.
  VARIANT 269: note = X at position 269 is different from T; in an embodiment, is an amino acid residue with a hydrophobic side chain (A, V, I, L, M, F, Y or W); and in yet another embodiment, is L. X at position 284 is any amino acid residue; in an embodiment, is different from A; in an embodiment, is an amino acid residue with a negatively-charged side chain (D or E); and in yet another embodiment, is D.
  VARIANT 284: note = X at position 284 is any amino acid residue; in an embodiment, is different from A; in an embodiment, is an amino acid residue with a negatively-charged side chain (D or E); and in yet another embodiment, is D.
  VARIANT 287: note = X at position 287 is different from A; in an embodiment, is an amino acid residue with a hydrophobic side chain (V, I, L, M, F, Y or W); and in yet another embodiment, is V.
  VARIANT 295: note = X at position 295 is different from R; in an embodiment, is P.
  VARIANT 313: note = X at position 313 is any amino acid residue; in an embodiment is different from P; in an embodiment, is an amino acid residue with a polar uncharged side chain (S, T, N or Q); and in yet another embodiment, is S.
  VARIANT 314: note = X at position 314 is different from M; in an embodiment, is an amino acid residue with a hydrophobic side chain (A, V, I, L, F, Y or W); in yet another embodiment, is I or L; in yet another embodiment, is I.
  VARIANT 327: note = X at position 327 is different from Q; in an embodiment, is an amino acid residue with a hydrophobic side chain (A, V, I, L, M, F, Y or W); in yet another embodiment, is L.
  VARIANT 374: note = X at position 374 is different from Q; in an embodiment, is an amino acid residue with a positively-charged side chain (R, K or H); and in yet another embodiment, is H.
  VARIANT 376: note = X at position 376 is different from A; in an embodiment, is an amino acid residue with a hydrophobic side chain (V, I, L, M, F, Y or W); in yet another embodiment, is V.
  VARIANT 384: note = X at position 384 is different from N; in an embodiment, is an amino acid residue with a hydrophobic side chain (A, V, I, L, M, F, Y or W); and in yet another embodiment, is I.
  VARIANT 389: note = X at position 389 is different from G; in an embodiment, is an amino acid residue with a negatively-charged side chain (D or E); in yet another embodiment is D.
  VARIANT 424: note = X at position 424 is different from P; in an embodiment, is an amino acid residue with a hydrophobic side chain (A, V, I, L, M, F, Y or W); and in yet another embodiment, is L.
  VARIANT 430: note = X at position 430 is different from G; in an embodiment, is C.
  VARIANT 468: note = X at position 468 is any amino acid residue; in an embodiment, is different from E; in another embodiment is an amino acid residue with a positively-charged side chain (R, K or H); and in yet another embodiment, is K.
  VARIANT 482: note = X at position 482 is any amino acid residue; in an embodiment is different from P; in an embodiment, is an amino acid residue with a polar uncharged side chain (S, T, N or Q); and in yet another embodiment, is S.
  VARIANT 487: note = X at position 487 is absent or present; when present, X at position 487 is different from A; in an embodiment, is an amino acid residue with a hydrophobic side chain (A, V, I, L, M, F, Y or W); and in yet another embodiment, is V.
  VARIANT 488: note = X at position 488 is absent or present; when present, X at position 488 is any amino acid residue; in an embodiment, is K.
  VARIANT 489: note = X at position 489 is absent or present; when present, X at position 489 is any amino acid residue; in an embodiment, is A.
  VARIANT 490: note = X at position 490 is absent or present; when present, X at position 490 is any amino acid residue; in an embodiment, is G.
FPVRRTDLGQXQGLAGDVXSFXGIPYAAPPVGXLRWKPPQXARPWAGVRPATQFGSDCFGAAYLRKGSLAPGVSEDCLYLNVWAPSXAKPGQYPVMVWVYGGGFAGGTAAMPYYDGEALARQGVVVVTXNYRTNIXGFFAHPGLSRESPTGTSGNYGLLDILAALRWVQSNAXAFGGDPGRVTVFGESAGASAIGLLLTSPLSKGLFRGAILESPGLTRPLATLADSAASGERLDADLSRLRSTDPATLMARADAARPASRDLRXPRPXGPIVDGHVLPQTDSXAIXAGQLAPVXVLIGTNADEGRAFLGRAXXETPADYQAYLEAXFGDQAAAVAACYPLDGRATPKEMVARIFGDNQFNRGVSAFSEALVRXGXPVWRYQFXGNTEXGRAPATHGAEIPYVFGVFKLDELGLFDWPPEGPTXADRALXQLMSSAWVRFAKNGDPAGDALTWPAYSTGKSTMTFGPXGRAAVVSPGPSIPXCADGXXXX

SEQ ID NO: 2 - protein, 490 aa, synthetic construct
  VARIANT 19: note = X at position 19 is any amino acid residue; in an embodiment is different from M; in another embodiment, is an amino acid residue with a hydrophobic side chain (A, V, L, I, F, Y or W); and in yet another embodiment, is I.
  VARIANT 33: note = X at position 33 is any amino acid residue; in an embodiment, is different from G; in another embodiment, is an amino acid residue with a negatively-charged side chain (D or E); and in yet another embodiment, is D.
  VARIANT 87: note = X at position 87 is different from G; in an embodiment, is an amino acid residue with a negatively-charged side chain (D or E); and in yet another embodiment, is D.
  VARIANT 136: note = X at position 136 is different from L; in an embodiment, is an amino acid residue with a hydrophobic side chain (A, V, I, M, F, Y or W); and in yet another embodiment, is F.
  VARIANT 265: note = X at position 265 is different from R, in an embodiment, is an amino acid residue with a positively-charged side chain (K or H); and in yet another embodiment, is K.
  VARIANT 269: note = X at position 269 is different from T; in an embodiment, is an amino acid residue with a hydrophobic side chain (A, V, I, L, M, F, Y or W); and in yet another embodiment, is L.
  VARIANT 284: note = X at position 284 is different from A; in an embodiment, is an amino acid residue with a negatively-charged side chain (D or E); and in yet another embodiment, is D.
  VARIANT 295: note = X at position 295 is different from R; in an embodiment, is P.
  VARIANT 314: note = X at position 314 is different from M; in an embodiment, is an amino acid residue with a hydrophobic side chain (A, V, I, L, F, Y or W); in yet another embodiment, is I or L; in yet another embodiment, is I.
  VARIANT 327: note = X at position 327 is different from Q; in an embodiment, is an amino acid residue with a hydrophobic side chain (A, V, I, L, M, F, Y or W); in yet another embodiment, is L.
  VARIANT 374: note = X at position 374 is different from Q; in an embodiment, is an amino acid residue with a positively-charged side chain (R, K or H); and in yet another embodiment, is H.
  VARIANT 376: note = X at position 376 is different from A; in an embodiment, is an amino acid residue with a hydrophobic side chain (V, I, L, M, F, Y or W); in yet another embodiment, is V.
  VARIANT 384: note = X at position 384 is different from N; in an embodiment, is an amino acid residue with a hydrophobic side chain (A, V, I, L, M, F, Y or W); and in yet another embodiment, is I.
  VARIANT 389: note = X at position 389 is different from G; in an embodiment, is an amino acid residue with a negatively-charged side chain (D or E); in yet another embodiment is D.
  VARIANT 430: note = X at position 430 is different from G; in an embodiment, is C.
  VARIANT 468: note = X at position 468 is different from E; in an embodiment is an amino acid residue with a positively-charged side chain (R, K or H); and in yet another embodiment, is K.
  VARIANT 482: note = X at position 482 is different from P; in an embodiment, is an amino acid residue with a polar uncharged side chain (S, T, N or Q); and in yet another embodiment, is S.
  VARIANT 487: note = X at position 487 is absent or present; when present, X at position 487 is different from A; in an embodiment, is an amino acid residue with a hydrophobic side chain (A, V, I, L, M, F, Y or W); and in yet another embodiment, is V.
  VARIANT 488: note = X at position 488 is absent or present; when present, X at position 488 is any amino acid residue; in an embodiment, is K.
  VARIANT 489: note = X at position 489 is absent or present; when present, X at position 489 is any amino acid residue; in an embodiment, is A.
  VARIANT 490: note = X at position 490 is absent or present; when present, X at position 490 is any amino acid residue; in an embodiment, is G.
FPVRRTDLGQVQGLAGDVXSFRGIPYAAPPVGXLRWKPPQHARPWAGVRPATQFGSDCFGAAYLRKGSLAPGVSEDCLYLNVWAPSXAKPGQYPVMVWVYGGGFAGGTAAMPYYDGEALARQGVVVVTFNYRTNIXGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRXPRPXGPIVDGHVLPQTDSXAIAAGQLAPVXVLIGTNADEGRAFLGRAPXETPADYQAYLEAXFGDQAAAVAACYPLDGRATPKEMVARIFGDNQFNRGVSAFSEALVRXGXPVWRYQFXGNTEXGRAPATHGAEIPYVFGVFKLDELGLFDWPPEGPTPADRALXQLMSSAWVRFAKNGDPAGDALTWPAYSTGKSTMTFGPEGRAAVVSPGPSIPXCADGXXXX

SEQ ID NO: 3 - protein, 490 aa, Caulobacter sp.
FPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRRPRPTGPIVDGHVLPQTDSAAIAAGQLAPVRVLIGTNADEGRAFLGRAPMETPADYQAYLEAQFGDQAAAVAACYPLDGRATPKEMVARIFGDNQFNRGVSAFSEALVRQGAPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADGAKAG

SEQ ID NO: 4 - protein, 490 aa, synthetic construct; identical to SEQ ID NO: 3

SEQ ID NO: 5 - protein, 486 aa, synthetic construct
FPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRRPRPTGPIVDGHVLPQTDSAAIAAGQLAPVRVLIGTNADEGRAFLGRAPMETPADYQAYLEAQFGDQAAAVAACYPLDGRATPKEMVARIFGDNQFNRGVSAFSEALVRQGAPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADG

SEQ ID NO: 6 - protein, 498 aa, synthetic construct
AGAATATDFPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRRPRPTGPIVDGHVLPQTDSAAIAAGQLAPVRVLIGTNADEGRAFLGRAPMETPADYQAYLEALFGDQAAAVAACYPLDGRATPKEMVARIFGDNQFNRGVSAFSEALVRQGVPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAIVSPGPSIPPCADGVKAG

SEQ ID NO: 7 - protein, 498 aa, synthetic construct
AGAATATDFPVRRTDLGQVQGLAGEVMSFXGIPYAAPPVGGLRWKPPQHAIPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVRVLIGTNADEGRAFLGRAPMETPADYQAYLEAQFGDQAAAVAACYPLDGRATPKEMVARIFGDNQFNRGVSAFSEALVRHGAPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADGAKAG

SEQ ID NO: 8 - protein, 490 aa, synthetic construct
FPVRRTDLGQVQGLAGEVMSFRGIPYAAPPVGGLRWKPPQHAIPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVRVLIGTNADEGRAFLGRAPMETPADYQAYLEALFGDQAAAVAACYPLDGRATPKEMVARIFGDNQFNRGVSAFSEALVRQGAPVWRYQFIGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADGAKAG

SEQ ID NO: 9 - protein, 490 aa, synthetic construct
FPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVRVLIGTNADEGRAFLGRAPMETPADYQAYLEALFGDQAAAVAACYPLDGRATPKEMVARIFGDNQFNRGVSAFSEALVRHGVPVWRYQFIGNTEGGRAPATHGAEIPYVFGVFKLDELGLFDWPPEGPTPADRALCQLMSSAWVRFAKNGDPAGDALTWPAYSTGKSTMTFGPEGRAAVVSPGPSIPPCADGVKAG

SEQ ID NO: 10 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVRVLIGTNADEGRAFLGRAPMETPADYQAYLEALFGDQAAAVAACYPLDGRATPKEMVARIFGDNQFNRGVSAFSEALVRHGVPVWRYQFIGNTEGGRAPATHGAEIPYVFGVFKLDELGLFDWPPEGPTPADRALCQLMSSAWVRFAKNGDPAGDALTWPAYSTGKSTMTFGPEGRAAVVSPGPSIPPCADGVKAG

SEQ ID NO: 11 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVSPGPSIPPCADGVKAG

SEQ ID NO: 12 - protein, 490 aa, synthetic construct
FPVRRTDLGQVQGLAGDVMSFKGIPYAAPPVGGLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPEGRAAVVSPGPSIPPCADGVKAG

SEQ ID NO: 13 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRASIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVSPGPSIPPCADGVKAG

SEQ ID NO: 14 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVSPGPSIPSCADGVKAG

SEQ ID NO: 15 - protein, 490 aa, synthetic construct
FPVRRTDLGQVQGLAGDVMSFKGIPYAAPPVGGLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSXAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVSPGPSIPSCADGVKAG

SEQ ID NO: 16 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVPVLIGTNADEGRAFLGRASIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVSPGPSIPPCADGVKAG

SEQ ID NO: 17 - protein, 490 aa, synthetic construct
FPVRRTDLGQVQGLAGDVMSFRGIPYAAPPVGGLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVXPGPSIPPCADXVKAG

SEQ ID NO: 18 - protein, 490 aa, synthetic construct
FPVRRTDLGQVQGLAGDVMSFKGIPYAAPPVGGLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVSPGPSIPSCADGVKAG

SEQ ID NO: 19 - protein, 490 aa, synthetic construct
FPVRRTDLGQVQGLAGDVISFKGIPYAAPPVGDLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPEGRAAVVSPGPSIPSCADGVKAG

SEQ ID NO: 20 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRASMETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVSPGPSIPPCADGVKAG

SEQ ID NO: 21 - protein, 490 aa, synthetic construct
FPVRRTDLGQVQGLAGDVISFKGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPEGRAAVVSPGPSIPSCADGVKAG

SEQ ID NO: 22 - protein, 490 aa, synthetic construct
FPVRRTDLGQVQGLAGDVISFKGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIVAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVSPGPSIPPCADGVKAG

SEQ ID NO: 23 - protein, 490 aa, synthetic construct
FPVRRTDLGQVQGLAGDVISFKGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSXAIAAGQLAPVPVLIGTNADEGRAFLGRAPLETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPEGRAAVVSPGPSIPSCADGVKAG

SEQ ID NO: 24 - protein, 490 aa, synthetic construct
FPVRRTDLGQVQGLAGDVMSFKGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIVAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVSPGPSIPPCADXVKAG

SEQ ID NO: 25 - protein, 490 aa, synthetic construct; identical to SEQ ID NO: 14

SEQ ID NO: 26 - protein, 490 aa, synthetic construct; identical to SEQ ID NO: 14

SEQ ID NO: 27 - protein, 490 aa, synthetic construct; identical to SEQ ID NO: 13

SEQ ID NO: 28 - protein, 490 aa, synthetic construct; identical to SEQ ID NO: 13

SEQ ID NO: 29 - protein, 490 aa, synthetic construct; identical to SEQ ID NO: 14

SEQ ID NO: 30 - protein, 490 aa, synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAIAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRASIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTPADRALCQLMSSAWVRFAKNGDPAGDALTWPAYSTGKSTMTFGPKGRAAVVXPGPSIPPCADGVKAG

SEQ ID NO: 31 - protein, 490 aa, synthetic construct; identical to SEQ ID NO: 11

SEQ ID NO: 32 - protein, 490 aa, synthetic construct; identical to SEQ ID NO: 14

SEQ ID NO: 33 - protein, 490 aa, synthetic construct
  VARIANT 19: note = X at position 19 is any amino acid residue; in an embodiment, is different from M; in another embodiment, is an amino acid residue with a hydrophobic side chain (A, V, L, I, F, Y or W); and in yet another embodiment, is I.
  VARIANT 22: note = X at position 22 is any amino acid residue; in an embodiment is different from R; in another embodiment, is an amino acid residue with a positively-charged side chain (K or H); and in yet another embodiment, is K.
  VARIANT 33: note = X at position 33 is any amino acid residue; in an embodiment is different from G; in another embodiment, is an amino acid residue with a negatively-charged side chain (D or E); and in yet another embodiment, is D.
  VARIANT 173: note = X at position 173 is any amino acid residue; in an embodiment is different from R; in another embodiment, is an amino acid residue with a hydrophobic side chain (A, V, I, L, M, F, Y or W); and in yet another embodiment, is I.
  VARIANT 284: note = X at position 284 is any amino acid residue; in an embodiment, is different from A; in an embodiment, is an amino acid residue with a negatively-charged side chain (D or E); and in yet another embodiment, is D.
  VARIANT 313: note = X at position 313 is any amino acid residue; in an embodiment is different from P; in an embodiment, is an amino acid residue with a polar uncharged side chain (S, T, N or Q); and in yet another embodiment, is S.
  VARIANT 468: note = X at position 468 is any amino acid residue; in an embodiment, is different from E; in another embodiment is an amino acid residue with a positively-charged side chain (R, K or H); and in yet another embodiment, is K.
  VARIANT 482: note = X at position 482 is any amino acid residue; in an embodiment is different from P; in an embodiment, is an amino acid residue with a polar uncharged side chain (S, T, N or Q); and in yet another embodiment, is S.
FPVRRTDLGQVQGLAGDVXSFXGIPYAAPPVGXLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNAXAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSXAIAAGQLAPVPVLIGTNADEGRAFLGRAXIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPXGRAAVVSPGPSIPXCADGVKAG

SEQ ID NO: 34 - protein, 87 aa, synthetic construct
MQLLRCFSIFSVIASVLAQELTTICEQIPSPTLESTPYSLSTTTILANGKAMQGVFEYYKSVTFVSNCDSHPSTTSKDSPINTQYVF

SEQ ID NO: 35 - protein, 502 aa, Caulobacter sp.
GASAAGAATATDFPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRRPRPTGPIVDGHVLPQTDSAAIAAGQLAPVRVLIGTNADEGRAFLGRAPMETPADYQAYLEAQFGDQAAAVAACYPLDGRATPKEMVARIFGDNQFNRGVSAFSEALVRQGAPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADGAKAG

SEQ ID NO: 36 - protein, 490 aa, synthetic construct
FPVRRTDLGQVQGLAGEVMSFRGIPYAAPPVGGLRWKPPQHAIPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVRVLIGTNADEGRAFLGRAPMETPADYQAYLEALFGDQAAAVAACYPLDGRATPKEMVARIFGDNQFNRGVSAFSEALVRQGVPVWRYQFIGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADGAKAG

SEQ ID NO: 37 - protein, 488 aa, synthetic construct
TDFPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRRPRPTGPIVDGHVLPQTDSAAIAAGQLAPVRVLIGTNADEGRAFLGRAPMETPADYQAYLEAQFGDQAAAVAACYPLDGRATPKEMVARIFGDNQFNRGVSAFSEALVRQGAPVWRYQFNGNTEGGRAPATHGAEIPYVFGVFKLDELGLFDWPPEGPTPADRALGQLMSSAWVRFAKNGDPAGDALTWPAYSTGKSTMTFGPEGRAAVVSPGPSIPPCADG

SEQ ID NO: 38 - protein, 490 aa, synthetic construct
FPVRRTDLGQVQGLAGDVMSFRGIPYAAPPVGGLRWKPPQHARPWAGVRPATQFGSDCFGAAYLRKGSLAPGVSEDCLYLNVWAPSGAKPGQYPVMVWVYGGGFAGGTAAMPYYDGEALARQGVVVVTFNYRTNILGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSAAIAAGQLAPVPVLIGTNADEGRAFLGRAPMETPADYQAYLEALFGDQAAAVAACYPLDGRATPKEMVARIFGDNQFNRGVSAFSEALVRHGVPVWRYQFIGNTEGGRAPATHGAEIPYVFGVFKLDELGLFDWPPEGPTPADRALCQLMSSAWVRFAKNGDPAGDALTWPAYSTGKSTMTFGPEGRAAVVSPGPSIPPCADGVKAG

SEQ ID NO: 39 - protein, 89 aa, Saccharomyces cerevisiae
MRFPSIFTAVLFAASSALAAPANTTTEDETAQIPAEAVIDYSDLEGDFDAAALPLSNSTNNGLSSTNTTIASIAAKEEGVQLDKREAEA

SEQ ID NO: 40 - protein, 502 aa, synthetic construct; identical to SEQ ID NO: 35

SEQ ID NO: 41 - protein, 34 aa, synthetic construct
MVDAKKVLSVTAGFVGAAGLATLATGANTVSAST

(19) United States
(12) Patent Application Publication
(10) Pub. No.: US 2025/0346873 A1
(43) Pub. Date: Nov. 13, 2025

(54) RECOMBINANT EXPRESSION OF FUMONISIN ESTERASE, COMPOSITIONS AND USES THEREOF

(71) Applicant: DANSTAR FERMENT AG, Zug (CH)

(72) Inventors: Allan FROEHLICH, Lebanon, NH (US); Shelly CHENG, Lebanon, NH (US); Erin WISWALL, Lebanon, NH (US); John MCBRIDE, Lebanon, NH (US)

(21) Appl. No.: 18/832,323

(22) PCT Filed: Jan. 26, 2023
(86) PCT No.: PCT/IB2023/050690

§ 371 Date: Jul. 23, 2024

Related U.S. Application Data
(60) Provisional application No. 63/303,394, filed on Jan. 26, 2022.

(51) Int. Cl. C12N 9/18

(52) U.S. Cl. CPC C12N 9/18; C12Y 301/01087

(57) ABSTRACT

The present disclosure concerns polypeptide having fumonisin esterase activity exhibiting increased fumonisin esterase activity, when measured at a temperature of at least 37° C., when compared to the control polypeptide comprising the amino acid of SEQ ID NO: 3. The polypeptides of the present disclosure include one or more amino acid variations which contribute to the increase fumonisin esterase activity. The present disclosure also includes compositions comprising the polypeptide having fumonisin esterase activity, methods for detoxifying a fumonisin mycotoxin as well as processes for making the compositions comprising the polypeptide having fumonisin esterase activity.

### CROSS-REFERENCE TO RELATED APPLICATION(S) AND DOCUMENT(S)

This application claims priority from U.S. provisional patent application 63/303,394 filed on Jan. 26, 2022 and herewith incorporated in its entirety. This application also comprises a sequence listing in electronic form which is also incorporated in its entirety.

### TECHNOLOGICAL FIELD

The present disclosure concerns polypeptides having fumonisin esterase activity which can be expressed in a recombinant microbial host cell.

### BACKGROUND

Fumonisins are secondary metabolites produced in cereals by pathogenic fungi, namely Fusarium verticillioides, Fusarium proliferatum, and related species. In addition, Aspergillus nigri also produces fumonisins in crops such as peanuts, maize and grapes. They predominantly contaminate corn and corn-based products and have also been detected on multiple grain-based products including oats, wheat, barley, as well as grapes. More than 15 homologues of fumonisins have been identified and characterized as fumonisins A, B, C and P. Of the fumonisins B, FB1, FB2 and FB3 are the most abundant, with FB1 being the most toxic form that can coexist with other forms of fumonisins, i.e. FB2 and FB3 and accounts for over 75% of contamination events. FB1 consists of a diester with propane-1,2,3-tricarballylic acid (TCA) and 2-amino-12,16-dimethyl-3,5,10,14,15-pentahydroxyleicosane where the hydroxyl (OH—) groups in the C-14 and C-15 position are involved with the carboxyl (—COOH) groups of the TCA to form an ester.

Enzymatic degradation of FB1 is the most efficient way to reduce its toxicity. The two tricarballylic acid moieties of FB1 can be sequentially cleaved off by esterases, which results in two intermediate products, partially hydrolyzed FB1-1 and 2 (namely pHFB1-1 and pHFB1-2, respectively). The final product, hydrolyzed FB1 (HFB1), is less harmful to animals than FB1. Fumonisin esterases do not necessarily exhibit enzymatic activity across a broad range of temperatures and pH which limits downstream processing options and applications.

There is thus a need for polypeptides or enzymes that exhibit suitable fumonisin remediation activity in various temperature and pH ranges, while being able to be administered or spread in several forms, i.e., purified, but also associated to the microbial host cell to cover all ranges of applications.

### SUMMARY

The present disclosure provides polypeptides exhibiting fumonisin esterase activity, which is increased, when measured at a temperature of at least 37° C., when compared to the control polypeptide comprising the amino acid of SEQ ID NO: 3.

According to a first aspect, the present disclosure provides a polypeptide having fumonisin esterase activity and comprising the amino acid sequence of SEQ ID NO: 1, wherein the amino residues at positions 487, 488, 489 and 490 are independently present or absent; and wherein at least one of: the amino acid residue at position 11 is different from a valine residue; the amino acid residue at position 19 is different from a methionine residue; the amino acid residue at position 22 is different from an arginine residue; the amino acid residue at position 33 is different from a glycine residue; the amino acid residue at position 41 is different from a histidine residue; the amino acid residue at position 87 is different from a glycine residue; the amino acid residue at position 129 is different from a phenylalanine residue; the amino acid residue at position 136 is different from a leucine residue; the amino acid residue at position 173 is different from an arginine residue; the amino acid residue at position 265 is different from an arginine residue; the amino acid residue at position 269 is different from a threonine residue; the amino acid residue at position 284 is different from an alanine residue; the amino acid residue at position 287 is different from an alanine residue; the amino acid residue at position 295 is different from an arginine residue; the amino acid residue at position 313 is different from a proline residue; the amino acid residue at position 314 is different from a methionine residue; the amino acid residue at position 327 is different from a glutamine residue; the amino acid residue at position 374 is different from a glutamine residue; the amino acid residue at position 376 is different from an alanine residue; the amino acid residue at position 384 is different from an asparagine residue; the amino acid residue at position 389 is different from a glycine residue; the amino acid residue at position 424 is different from a proline residue; the amino acid residue at position 430 is different from a glycine residue; the amino acid residue at position 468 is different from a glutamic acid residue; the amino acid residue at position 482 is different from a proline residue; or the amino acid residue at position 487, when present, is different from an alanine residue; and wherein the polypeptide has increased residual fumonisin esterase activity, when measured after a heat challenge at a temperature of at least 37° C. for at least 1 minute, when compared to the control polypeptide comprising the amino acid of SEQ ID NO: 3. In an embodiment, the polypeptide of claim 1, wherein at least one of: the amino acid residue at position 11 comprises a hydrophobic side chain; the amino acid residue at position 19 comprises a hydrophobic side chain; the amino acid residue at position 22 comprises a positively-charged side chain; the amino acid residue at position 33 comprises a negatively-charged side chain; the amino acid residue at position 41 comprises a polar uncharged side chain; the amino acid residue at position 87 comprises a negatively-charged side chain; the amino acid residue at position 129 comprises a hydrophobic side chain; the amino acid residue at position 136 comprises a hydrophobic side chain; the amino acid residue at position 173 comprises a hydrophobic side chain; the amino acid residue at position 265 comprises a positively-charged side chain; the amino acid residue at position 269 comprises a hydrophobic side chain; the amino acid residue at position 284 comprises a negatively-charged side chain; the amino acid residue at position 287 comprises a hydrophobic side chain; the amino acid residue at position 313 comprises a polar uncharged side chain; the amino acid residue at position 314 comprises a hydrophobic side chain; the amino acid residue at position 327 comprises a hydrophobic side chain; the amino acid residue at position 374 comprises a positively-charged side chain; the amino acid residue at position 376 comprises a hydrophobic side chain; the amino acid residue at position 384 comprises a hydrophobic side chain; the amino acid residue at position 389 comprises a negatively-charged side chain; the amino acid residue at position 424 comprises a hydrophobic side chain; the amino acid residue at position 468 comprises a positively-charged side chain; the amino acid residue at position 482 comprises a polar uncharged side chain; or the amino acid residue at position 487, when present, comprises a hydrophobic side chain. In an embodiment, in the polypeptide, wherein at least one of: the amino acid residue at position 11 is a leucine residue; the amino acid residue at position 19 is an isoleucine residue; the amino acid residue at position 22 is a lysine residue; the amino acid residue at position 33 is an aspartic acid residue; the amino acid residue at position 41 is a glutamine residue; the amino acid residue at position 87 is an aspartic acid residue; the amino acid residue at position 129 is a tyrosine residue; the amino acid residue at position 136 is a phenylalanine residue; the amino acid residue at position 173 is an isoleucine residue; the amino acid residue at position 265 is a lysine residue; the amino acid residue at position 269 is a leucine residue; the amino acid residue at position 284 is an aspartic acid residue; the amino acid residue at position 287 is a valine residue; the amino acid residue at position 295 is a proline residue; the amino acid residue at position 313 is a serine residue; the amino acid residue at position 314 is a leucine or an isoleucine residue; the amino acid residue at position 327 is a leucine residue; the amino acid residue at position 374 is a histidine residue; the amino acid residue at position 376 is a valine residue; the amino acid residue at position 384 is an isoleucine residue; the amino acid residue at position 389 is an aspartic acid residue; the amino acid residue at position 424 is a leucine residue; the amino acid residue at position 430 is a cysteine residue; the amino acid residue at position 468 is a lysine residue; the amino acid residue at position 482 is a serine residue; or the amino acid residue at position 487, when present, is a valine residue. In a further embodiment, in the polypeptide, the amino acid residue at position 314 is an isoleucine residue. In a further embodiment, the polypeptide has the amino acid sequence of one of SEQ ID NO: 6 to 32 or 40. In yet another embodiment, the polypeptide comprises the amino acid sequence of SEQ ID NO: 2, wherein the amino residues at positions 487, 488, 489 and 490 are independently present or absent; and wherein at least one of: the amino acid residue at position 19 is different from a methionine residue; the amino acid residue at position 33 is different from a glycine residue; the amino acid residue at position 87 is different from a glycine residue; the amino acid residue at position 136 is different from a leucine residue; the amino acid residue at position 265 is different from an arginine residue; the amino acid residue at position 269 is different from a threonine residue; the amino acid residue at position 284 is different from an alanine residue; the amino acid residue at position 295 is different from an arginine residue; the amino acid residue at position 314 is different from a methionine residue; the amino acid residue at position 327 is different from a glutamine residue; the amino acid residue at position 374 is different from a glutamine residue; the amino acid residue at position 376 is different from an alanine residue; the amino acid residue at position 384 is different from an asparagine residue; the amino acid residue at position 389 is different from a glycine residue; the amino acid residue at position 430 is different from a glycine residue; the amino acid residue at position 482 is different from a proline residue; or the amino acid residue at position 487, when present is different from an alanine residue. In an embodiment, in the polypeptide, at least one of: the amino acid residue at position 19 comprises a hydrophobic side chain; the amino acid residue at position 33 comprises a negatively-charged side chain; the amino acid residue at position 87 comprises a negatively-charged side chain; the amino acid residue at position 136 comprises a hydrophobic side chain; the amino acid residue at position 265 comprises a positively-charged side chain; the amino acid residue at position 269 comprises a hydrophobic side chain; the amino acid residue at position 284 comprises a negatively-charged side chain; the amino acid residue at position 314 comprises a hydrophobic side chain; the amino acid residue at position 327 comprises a hydrophobic side chain; the amino acid residue at position 374 comprises a positively-charged side chain; the amino acid residue at position 376 comprises a hydrophobic side chain; the amino acid residue at position 384 comprises a hydrophobic side chain; the amino acid residue at position 389 comprises a negatively-charged side chain; the amino acid residue at position 482 comprises a polar uncharged side chain; or the amino acid residue at position 487, when present, comprises a hydrophobic side chain. In yet another embodiment, in the polypeptide, at least one of: the amino acid residue at position 19 is an isoleucine residue; the amino acid residue at position 33 is an aspartic acid residue; the amino acid residue at position 87 is an aspartic acid residue; the amino acid residue at position 136 is a phenylalanine residue; the amino acid residue at position 265 is a lysine residue; the amino acid residue at position 269 is a leucine residue; the amino acid residue at position 284 is an aspartic acid residue; the amino acid residue at position 295 is a proline residue; the amino acid residue at position 314 is a leucine or an isoleucine residue; the amino acid residue at position 327 is a leucine residue; the amino acid residue at position 374 is a histidine residue; the amino acid residue at position 376 is a valine residue; the amino acid residue at position 384 is an isoleucine residue; the amino acid residue at position 389 is an aspartic acid residue; the amino acid residue at position 430 is a cysteine residue; the amino acid residue at position 482 is a serine residue; or the amino acid residue at position 487, when present, is a valine residue. In still another embodiment, the amino acid residue at position 314 is an isoleucine residue. In yet another embodiment, the polypeptide comprises the amino acid sequence of SEQ ID NO: 33 and the amino acid residue at position 19 is a methionine or an isoleucine residue; the amino acid residue at position 22 is a lysine or an arginine residue; the amino acid residue at position 33 is a glycine or a glutamic acid residue; the amino acid residue at position 173 is an arginine or an isoleucine residue; the amino acid residue at position 284 is an alanine or an aspartic acid residue; the amino acid residue at position 313 is a proline or a serine residue; the amino acid residue at position 468 is a glutamic acid or a lysine residue; and the amino acid residue at position 482 is a proline or a serine residue. In still a further embodiment, the polypeptide has the one of the amino acid sequences of SEQ ID NO: 11, 12, 13 or 14.

According to a second aspect, the present disclosure provides a variant of the polypeptide as described herein (i) having at least 70% identity with the amino acid sequence of SEQ ID NO: 1, and (ii) exhibiting increased residual fumonisin esterase activity, when measured after a heat challenge at a temperature of at least 37° C. for at least 1 minute, when compared to the control polypeptide comprising the amino acid of SEQ ID NO: 3.

According to a third aspect, the present disclosure provides a fragment of the polypeptide and/or the variant as described herein (i) having at least 70% identity with the amino acid sequence of SEQ ID NO: 1 and (ii) exhibiting increased residual fumonisin esterase activity, when measured after a heat challenge at a temperature of at least 37° C. for at least 1 minute, when compared to the control polypeptide comprising the amino acid of SEQ ID NO: 3.

According to a fourth aspect, the present disclosure provides a recombinant microbial host cell comprising a heterologous nucleic acid molecule encoding the polypeptide having fumonisin esterase activity, the variant or the fragment as described herein. In an embodiment, the heterologous nucleic acid molecule encoding the polypeptide having fumonisin esterase activity as described herein, allows the expression of an intracellular form of the polypeptide having fumonisin esterase activity; a tethered form of the polypeptide having fumonisin esterase activity; or a secreted form of the polypeptide having fumonisin esterase activity. In an embodiment, the recombinant microbial host cell as described herein is a yeast host cell, a fungal host cell or a bacterial host cell.

According to a fifth aspect, the present disclosure provides a composition comprising (i) at least one polypeptide having fumonisin esterase activity, at least one variant and/or at least one fragment as described herein and (ii) a further component. In an embodiment, the further component is or comprises a mineral and/or an organic binder. In another embodiment, the further component is or comprises a polypeptide having the ability to detoxify a mycotoxin. In another embodiment, the further component is or comprises a further polypeptide having fumonisin esterase activity, a further variant and/or a further fragment as described herein. In another embodiment, the further component is or comprises a microbe or a component of a microbe. In yet another embodiment, the microbe is the recombinant microbial host cell or a component of the recombinant microbial host cell as described herein. In another embodiment, the further component is or comprises a food ingredient, a feed ingredient, a beverage ingredient and/or a biomass to produce biofuel.

According to a sixth aspect, the present disclosure provides a method of detoxifying a fumonisin mycotoxin, the method comprising contacting the polypeptide having fumonisin esterase activity, the variant and/or the fragment as described herein, the recombinant microbial host cell and/or the composition as described herein with the fumonisin mycotoxin so as to cause the hydrolysis of at least one tricarballylic acid moiety from the fumonisin mycotoxin. In an embodiment, the fumonisin mycotoxin is suspected to be present in the composition and the method further comprises fermenting the composition. In an embodiment, the fumonisin mycotoxin is suspected to be present in the composition and the method further comprises administering the polypeptide having fumonisin esterase activity, the variant or the fragment as described herein, the recombinant microbial host cell and/or the composition as described herein, to a subject. In another embodiment, the subject is an animal. In yet another embodiment, the subject is a human. In yet another embodiment, the subject is a ruminant, a swine, an equine, a canine, a feline, a fish, a bird, or an aquatic animal. In an embodiment, the fumonisin mycotoxin is suspected to be present in a food ingredient, a feed ingredient and/or a beverage ingredient and the method further comprises making a food product, a feed product and/or a beverage. In yet another embodiment, the food ingredient, the feed ingredient and/or the beverage ingredient is or comprises corn. In yet another embodiment, the feed product is or comprises silage, hay, straw, grains, grain by-products, legumes, cottonseed meal, vegetables, milk and/or milk by-products. In yet another embodiment, the food product is or comprises a flour.

According to a seventh aspect, the present disclosure provides a process for making a composition comprising the polypeptide having fumonisin esterase activity, the variant and/or the fragment described herein, the process comprising a) propagating the recombinant microbial host cell as described herein to obtain a propagated recombinant microbial host cell and the polypeptide having fumonisin esterase activity, the variant and/or the fragment; b) the process optionally comprises separating the propagated microbial host cell from the polypeptide having fumonisin esterase activity, the variant or the fragment from at least one component of the propagated recombinant microbial host cell to obtain a separated fraction enriched in the polypeptide having fumonisin esterase activity, the variant or the fragment; inactivating the propagated microbial host cell to obtain an inactivated fraction; lysing the propagated microbial host cell to obtain a lysed fraction; drying, at least in part, the propagated microbial host cell, the separated fraction, the inactivated fraction or the lysed fraction to obtain a dried fraction; c) the process optionally comprises substantially purifying the polypeptide having fumonisin esterase activity, the variant or the fragment from the separated fraction, the inactivated fraction, the lysed fraction or the dried fraction to obtain a purified fraction; and d) the process comprises formulating the propagated recombinant microbial host cell, the optionally separated fraction, the optionally inactivated fraction, the optionally lysed fraction, the optionally dried fraction, or the optionally substantially purified fraction into the composition. In an embodiment, the optionally dried fraction of step b) is obtained by roller-drying, spray-drying, freeze-drying, electrospray-drying or fluid-bed drying of the separated fraction, the inactivated fraction, the lysed fraction or the dried fraction microbial host cell. In another embodiment, the propagated recombinant microbial cell, the separated fraction, the inactivated fraction, the lysed fraction, the optionally dried fraction, or the substantially purified fraction into the composition of step d) are pelleted, optionally in combination with a feed ingredient. In yet another embodiment, the feed pellet of step d), obtained with the dissociated fraction, the inactivated fraction, the lysed fraction, the optionally dried fraction, or the substantially purified fraction into the composition is coated. In yet another embodiment, step d) further comprises applying a heat treatment.

### BRIEF DESCRIPTION OF THE DRAWINGS

FIG. 1 shows a schematic representation of fumonisin (FB1) degradation by the polypeptides having a fumonisin esterase activity (FE). FB1 is partially hydrolyzed into pHFB1-1 or pHFB1-2, each hydrolysis releasing a tricarballylic acids (TCA) moiety. The completion of the hydrolysis reaction ultimately gives the hydrolyzed fumonisin (HFB) and two TCA moieties.

FIG. 2A compares the FE activity associated intracellular, tethered, and secreted FE-expressing strains. The results are presented as the amount of each of FB1 (black bars), pHFB1-1 (gray bars), pHFB1-2 (white bars) and HFB1 (dotted bars) (presented as LCMS peak area×100000) in function of the strains characterized (M10580 (negative control), M22810 (intracellular FE), M22929 (tethered FE) and M20435 (secreted FE)).

FIG. 2B compares the amount, as determined by LCMS, of FB1 (black bars), pHFB1-1 (grey bars), pHFB1-2 (white bars) and HFB1 (dotted bars) present in the supernatant associated with strains M20435 as well as transformants T8612-1, -2, -3 and -4, compared to a control (buffer only).

FIGS. 3A and 3B compare the amount, as determined by LCMS, of FB1 (first bars), pHFB1-1 (second bars), pHFB1-2 (third bars) and HFB1 (fourth bars) in an assay combining FB1 with culture supernatants from S. cerevisiae strains secreting different fumonisin esterase polypeptides. (A) The supernatant of strain M10580 (a control strain that does not express a polypeptide having fumonisin esterase activity) was compared to the supernatant of a strain expressing 102101F5, 112502A10 or 113002H1 following treatment of the supernatants for 5 min at temperatures between 56.1 and 64° C. (B) The supernatant of strain M10580 (a control strain that does not express a polypeptide having fumonisin esterase activity) was compared to the supernatant of strain M23401 strain as well as to a strain expressing 112502A10 following treatment of the supernatants for 5 min at temperatures between 52.9 and 63.8° C.

FIGS. 4A and 4B compare the amount (A) as well as the activity (B) of the polypeptides having fumonisin esterase activity in the supernatant of various strains. (A) Results are shown as the amount, as determined by Western blot, of the polypeptide having fumonisin esterase activity (in mg/mL) as a function of strain used (M20435 and M23401). (B) Results are shown for the amount, as determined by LCMS, of FB1 (black bars), pHFB1-1 (grey bars), pHFB1-2 (white bars) and HFB1 (dotted bars) present in an assay combining FB1 with culture supernatants associated with strains M10580, M20435 and M23401.

FIGS. 5A and 5B compare the amount, as determined by Western blot, of polypeptides having FE activity (A) as well as the % of residual fumonisin esterase activity, as determined by LCMS, after a heat challenge (B) present in the supernatant of various strains. (A) Results are shown as the amount of the polypeptide having fumonisin esterase activity (in mg/ml) as a function of strain used (M23283, M23401, M23399 and M23849). (B) Results are shown as % of residual fumonisin esterase activity as a function of the temperature of the heat challenge and the type of strains used (M20435 (expressing the wt FE3), M23399 and M23849).

FIG. 6 shows the amounts of FB1 and hFB1 quantified in high-moisture corn silage samples after FE (SEQ IS NO: 14) treatment. Samples A (A1, A2 and A3) represent negative control with no FE added, whereas Samples B (B1, B2 and B3) and C (C1, C2 and C3) correspond to a treatment with 40 and 80 U FE/kg of corn silage, respectively. The results are shown as the ppm of FB1 (stapled bars) and hFB1 (grey bars) for each Sample.

FIGS. 7A and 7B represent an extracted ion chromatograms for FB1, FB2, FB3, HFB1, HFB2 and HFB3 in (A) Sample A2 (negative control), and in (B) Sample B2 (treated by FE of SEQ ID NO: 14, 40 U/kg).

FIG. 8 provides a schematic representation for making an inactivated product comprising polypeptides having fumonisin esterase activity. Strain M27750 (expressing a polypeptide having the amino acid sequence of SEQ ID NO: 14) was propagated at 20 L scale using methods that would be deployed at a larger commercial scale to generate yeast biomass and enable FE secretion into the supernatant. A separator (centrifuge) was used to separate the yeast biomass (commercial yeast) from the liquid supernatant (commercial first beer storage) containing the FE enzyme. The yeast biomass was washed to remove any residual supernatant from the propagation and then the cells were lysed. The resulting yeast cell debris was pelleted by centrifugation and the released internal cytoplasmic contents found in the supernatant were used in a TCA assay to measure FE activity released via the cell lysis. The histogram presents the FE enzymatic activity (U/g of DCW) in function of individual propagations (H140621, H150621, or H160621) or as an average of these propagations.

FIG. 9 shows the relative amount of FB1, pHFB1, and HFB1 in corn mash fermentations at 66 hours. Results are shown as percentage of FB1 remaining in the mash relative to the No FE-producing yeast added sample in function of the type of fermenting S. cerevisiae strain used (M2390 (wild-type biofuel strain) and/or M23192 (engineered to secrete the polypeptide having the amino acid sequence of SEQ ID NO: 14)) or the amount of spray dried FE enzyme added, as well as the presence or absence of fumonisin. A=M2390 only, no fumonisin added; B=100% M2390 with fumonisin; C=99% M2390, 1% M23192, fumonisin added; D=98% M2390, 2% M23192, fumonisin added; E=95% M2390, 5% M23192, fumonisin added; F=90% M2390, 10% M23192, fumonisin added; G=M2390 only, fumonisin added, 31 mg of purified enzyme added; H=M2390 only, fumonisin added, 15 mg of purified enzyme added; I=M2390 only, fumonisin added, 7 mg of purified enzyme added; J=M2390 only, fumonisin added, 7 mg of purified enzyme added.

FIG. 10 compares the amount of FE activity remaining following polypeptide having a fumonisin esterase activity (having the amino acid sequence of SEQ ID NO: 14) inclusion in feed pellets produced at 60, 70, or 80° C. The results are presented as TCA activity (U per Kg of pig meal). Same preparation that did not undergo pelleting is included as a control (No pelleting).

FIG. 11 compares the amount, as determined by LCMS, of FB1 (black bars), pHFB1-1 (grey bars), pHFB1-2 (white bars) and HFB1 (dotted bars) present in the culture medium and in the supernatant associated with L. casei strains 4657 (control) and 4616 (SEQ ID NO: 40, FE3 variant), both compared to uncultured medium only (MRS+FUM).

### DETAILED DESCRIPTION

### Polypeptides Having Fumonisin Esterase Activity, Associated Variants and Fragments

The present disclosure relates to polypeptides exhibiting fumonisin esterase activity to allow the detoxification of fumonisins, especially fumonisins bearing at least one or two tricarballylic ester substituents. Such polypeptides possess, compared to the native enzyme, an increased activity due to i) their optimized secretion rate and ii) their increased stability to cope with the pH and temperature conditions encountered in a wide range of applications and processing conditions. In some embodiments, the polypeptides exhibiting fumonisin esterase activity of the present disclosure are intended to be expressed in a recombinant microbial host cell. The polypeptides can be provided from a recombinant microbial host cell or a composition or a product obtained from the recombinant microbial host cell.

The polypeptides of the present disclosure have fumonisin esterase activity and are carboxylic ester hydrolases, also referred as acylhydrolase or esterases. Polypeptides having esterase activity (EC 3.1.1) catalyze the hydrolysis of at least one ester linkage from the considered substrate. Polypeptides having fumonisin esterase activity include, as a substrate, fumonisin. In some embodiments, polypeptides having fumonisin esterase activity include, as a substrate, a fumonisin bearing at least one tricarballylic ester substituent. In some other embodiments, polypeptides having fumonisin esterase activity include, as a substrate, a fumonisin produced by the mold fungus Fusarium sp. as well as derivatives and degradation products thereof, yet in particular to fumonisins A1-2 (FA1-2), fumonisins B1-4 (FB1-4), fumonisins C1, 2, 4 (FC1, FC2, FC4) and to partially hydrolyzed fumonisins pHA1-2, pHFB1-4 and pHFC1-2. In some preferred embodiments, polypeptides having fumonisin esterase activity include, as a substrate, fumonisins B1, B2 and B3 and their partially hydrolyzed counterparts pHB1, pHB2 and pHB3, respectively.

In one embodiment, the present disclosure provides polypeptides exhibiting fumonisin esterase activity which is increased when compared to the control polypeptide comprising the amino acid sequence of SEQ ID NO: 3. The polypeptides of the present disclosure include at least one amino acid modification (substitution, deletion, addition) when compared to the amino acid sequence of SEQ ID NO: 3. In an embodiment, the polypeptides having fumonisin esterase activity which is increased when compared to the control polypeptide comprising the amino acid of SEQ ID NO: 3 is a variant of the polypeptides comprising the amino acid sequences of SEQ ID NO: 1, SEQ ID NO: 2 or SEQ ID NO: 33 and/or a fragment of the polypeptides comprising the amino acid sequences of SEQ ID NO: 1, SEQ ID NO: 2 or SEQ ID NO: 33.

The present disclosure also provides a variant of the polypeptides having the amino acid sequence of SEQ ID NO: 1, 2 or 33. As used in the context of the present disclosure, a “variant” includes at least one amino acid difference when compared to the amino acid sequence of SEQ ID NO: 1, 2 or 33. It is understood that the variants and fragments described herein include the substitutions described for SEQ ID NO: 1, 2 or 33. The present disclosure also provides a fragment of the polypeptides having the amino acid sequence of SEQ ID NO: 1, 2 or 33 or its associated variants. As used in the context of the present disclosure, a “fragment” includes at least one deleted amino acid residues when compared to the amino acid sequence of SEQ ID NO: 1, 2 or 33 or its associated fragments. In some embodiments, the variants and the fragments can also have at least 70%, 80%, 85%, 90%, 95%, 96%, 97%, 98% or 99% identity to the amino acid sequence of SEQ ID NO: 1, 2 or 33 (including the full-length of the amino acid sequence of SEQ ID NO: 1, 2 or 33). The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant fumonisin esterase polypeptides described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions of an amino acid by another one belonging to same category determined by its side chain: within amino acids presenting hydrophobic side chain (Alanine, Valine, Isoleucine, Leucine, Methionine, Phenylalanine, Tyrosine or Tryptophan); within amino acids presenting positively charged side chain (Arginine, Histidine or Lysine); negatively charged side chain (Aspartic acid or Glutamic acid) and polar-uncharged side chain (Serine, Threonine, Asparagine or Glutamine); or substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant fumonisin esterase polypeptide can also be a conservative variant or an allelic variant. As used herein, a conservative variant refers to alterations in the amino acid sequence that do not adversely affect the biological function(s) of the polypeptide having fumonisin esterase activity (e.g., detoxification of fumonisin). A substitution, insertion or deletion is said to adversely affect the polypeptide when the altered sequence prevents or disrupts a biological function associated with the polypeptide (e.g., the ester linkage hydrolysis of a fumonisin substrate). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the fumonisin esterase.

A fragment polypeptide can correspond to the polypeptides having fumonisin esterase activity described herein to which the signal peptide sequence has been removed. In other embodiments, the fragment polypeptide having fumonisin esterase activity can be, for example, a truncation of one or more, two or more, three or more, four or more amino acid residues at the N-terminal extremity of the non-truncated polypeptide or variant. In other embodiments, the fragment polypeptide having fumonisin esterase activity can be, for example, a truncation of one or more, two or more, three or more, four or more, five or more, six or more, seven or more, eight or more, nine or more, ten or more, eleven or more, twelve or more amino acid residues at the C-terminal extremity of the non-truncated polypeptide or variant. In other embodiments, the fragment polypeptide having fumonisin esterase activity can be, for example, a truncation of both termini of the polypeptide having fumonisin esterase activity or variant, wherein one or more, two or more, three or more, four or more amino acid residues are truncated at the N-terminal extremity and wherein one or more, two or more, three or more, four or more, five or more, six or more, seven or more, eight or more, nine or more, ten or more, eleven or more, twelve or more amino acid residues are truncated at the C-terminal extremity, when compared to the non-truncated polypeptide or variant. Alternatively or in combination, the fragment can be generated from removing one or more internal amino acid residues. In an embodiment, the fragment of the polypeptide having fumonisin esterase activity has at least 100, 150, 200, 250, 300, 350, 400, 450 or more consecutive amino acids of the variant polypeptide having increased fumonisin esterase activity.

In an embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1. In the polypeptides having the amino acid sequence of SEQ ID NO: 1, the amino acid residues at positions 487, 488, 489 and 490 are independently present or absent. In some embodiments of the polypeptides having the amino acid sequence of SEQ ID NO: 1, the amino acid residue at position 487 is present. In some embodiments of the polypeptides having the amino acid sequence of SEQ ID NO: 1, the amino acid residue at position 487 is absent. In some embodiments of the polypeptides having the amino acid sequence of SEQ ID NO: 1, the amino acid residue at position 488 is present. In some embodiments of the polypeptides having the amino acid sequence of SEQ ID NO: 1, the amino acid residue at position 488 is absent. In some embodiments of the polypeptides having the amino acid sequence of SEQ ID NO: 1, the amino acid residue at position 489 is present. In some embodiments of the polypeptides having the amino acid sequence of SEQ ID NO: 1, the amino acid residue at position 489 is absent. In some embodiments of the polypeptides having the amino acid sequence of SEQ ID NO: 1, the amino acid residue at position 490 is present. In some embodiments of the polypeptides having the amino acid sequence of SEQ ID NO: 1, the amino acid residue at position 490 is absent. In yet additional embodiments, the amino acid residue at positions 487, 488, 489 and 490 are present.

In the polypeptides having the amino acid sequence of SEQ ID NO: 1, some positions can be substituted by one or more amino acid residues. More specifically, in the polypeptides having the amino acid sequence of SEQ ID NO: 1, at least one of the following amino acid residue substitutions is present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least two of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least three of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least four of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least five of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least six of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least seven of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least eight of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least nine of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least ten of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least eleven of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least twelve of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least thirteen of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least fourteen of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least fifteen of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least sixteen of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, at least seventeen of any the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; or the amino acid residue at position 487, when present, is different from an alanine (A) residue. In some embodiments, in the polypeptides of the having the amino acid sequence of SEQ ID NO: 1, the following amino acid residue substitutions are present: the amino acid residue at position 11 is different from a valine (V) residue; the amino acid residue at position 41 is different from a histidine (H) residue; the amino acid residue at position 87 is different from a glycine (G) residue; the amino acid residue at position 129 is different from a phenylalanine (F) residue; the amino acid residue at position 136 is different from a leucine (L) residue; the amino acid residue at position 265 is different from an arginine (R) residue; the amino acid residue at position 269 is different from a threonine (T) residue; the amino acid residue at position 287 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 424 is different from a proline (P) residue; the amino acid residue at position 430 is different from a glycine (G) residue; and the amino acid residue at position 487, when present, is different from an alanine (A) residue.

In a further embodiment, the polypeptides comprising or consisting essentially of the amino acid sequence of SEQ ID NO: 1, at least one of the following amino acid residue substitutions is present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet another embodiment, the polypeptides comprising or consisting essentially of the amino acid sequence of SEQ ID NO: 1, at least two of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still another embodiment, the polypeptides comprising or consisting essentially of the amino acid sequence of SEQ ID NO: 1, at least three of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet a further embodiment, the polypeptides comprising or consisting essentially of the amino acid sequence of SEQ ID NO: 1, at least four of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still another embodiment, the polypeptides comprising or consisting essentially of the amino acid sequence of SEQ ID NO: 1, at least five of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet another embodiment, the polypeptides comprising or consisting essentially of the amino acid sequence of SEQ ID NO: 1, at least six of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In another embodiment, the polypeptides comprising or consisting essentially of the amino acid sequence of SEQ ID NO: 1, at least seven of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still a further embodiment, the polypeptides comprising or consisting essentially of the amino acid sequence of SEQ ID NO: 1, the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; and the amino acid residue at position 482 is different from a proline (P) residue.

In an embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2. In the polypeptides having the amino acid sequence of SEQ ID NO: 2, the amino acid residues at positions 487, 488, 489 and 490 are independently present or absent. In some embodiments of the polypeptides having the amino acid sequence of SEQ ID NO: 2, the amino acid residue at position 487 is present. In some embodiments of the polypeptides having the amino acid sequence of SEQ ID NO: 2, the amino acid residue at position 487 is absent. In some embodiments of the polypeptides having the amino acid sequence of SEQ ID NO: 2, the amino acid residue at position 488 is present. In some embodiments of the polypeptides having the amino acid sequence of SEQ ID NO: 2, the amino acid residue at position 488 is absent. In some embodiments of the polypeptides having the amino acid sequence of SEQ ID NO: 2, the amino acid residue at position 489 is present. In some embodiments of the polypeptides having the amino acid sequence of SEQ ID NO: 2, the amino acid residue at position 489 is absent. In some embodiments of the polypeptides having the amino acid sequence of SEQ ID NO: 2, the amino acid residue at position 490 is present. In some embodiments of the polypeptides having the amino acid sequence of SEQ ID NO: 2, the amino acid residue at position 490 is absent. In yet additional embodiments, the amino acid residue at positions 487, 488, 489 and 490 are present.

In the polypeptides having the amino acid sequence of SEQ ID NO: 2, some positions can be substituted by one or more amino acid residues. More specifically, in the polypeptides having the amino acid sequence of SEQ ID NO: 2, at least one of the following amino acid residue substitutions is present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least two of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least three of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least four of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least five of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least six of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least seven of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least eight of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least nine of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least ten of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least eleven of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least twelve of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least thirteen of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, at least fourteen of any the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; or the amino acid residue at position 487, when present, is different from an alanine (A) residue. In some embodiments, in the polypeptides of the having the amino acid sequence of SEQ ID NO: 2, the following amino acid residue substitutions are present: the amino acid residue at position 87 is different from a glycine (G) residue; the amino acid residue at position 136 is different from a leucine (L) residue; the amino acid residue at position 265 is different from an arginine (R) residue; the amino acid residue at position 269 is different from a threonine (T) residue; the amino acid residue at position 284 is different from an alanine (A) residue; the amino acid residue at position 295 is different from an arginine (R) residue; the amino acid residue at position 314 is different from a methionine (M) residue; the amino acid residue at position 327 is different from a glutamine (Q) residue; the amino acid residue at position 374 is different from a glutamine (Q) residue; the amino acid residue at position 376 is different from an alanine (A) residue; the amino acid residue at position 384 is different from an asparagine (N) residue; the amino acid residue at position 389 is different from a glycine (G) residue; the amino acid residue at position 430 is different from a glycine (G) residue; the amino acid residue at position 482 is different from a proline (P) residue; and the amino acid residue at position 487, when present, is different from an alanine (A) residue.

In a further embodiment, the polypeptides comprising or consisting essentially of the amino acid sequence of SEQ ID NO: 2, at least one of the following amino acid residue substitutions is present: The amino acid residue at position 19 is different from a methionine (M) residue or the amino acid residue at position 33 is different from a glycine (G) residue. In still a further embodiment, the polypeptides comprising or consisting essentially of the amino acid sequence of SEQ ID NO: 2, the following amino acid residue substitutions are present: The amino acid residue at position 19 is different from a methionine (M) residue and the amino acid residue at position 33 is different from a glycine (G) residue.

In an embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 33. In the polypeptides having the amino acid sequence of SEQ ID NO: 33, some positions can be substituted by one or more amino acid residues. More specifically, in the polypeptides having the amino acid sequence of SEQ ID NO: 33, at least one of the following amino acid residue substitutions is present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet another embodiment, the polypeptides comprising or consisting essentially of the amino acid sequence of SEQ ID NO: 33, at least two of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still another embodiment, the polypeptides comprising or consisting essentially of the amino acid sequence of SEQ ID NO: 33, at least three of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet a further embodiment, the polypeptides comprising or consisting essentially of the amino acid sequence of SEQ ID NO: 33, at least four of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still another embodiment, the polypeptides comprising or consisting essentially of the amino acid sequence of SEQ ID NO: 33, at least five of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In yet another embodiment, the polypeptides comprising or consisting essentially of the amino acid sequence of SEQ ID NO: 33, at least six of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In another embodiment, the polypeptides comprising or consisting essentially of the amino acid sequence of SEQ ID NO: 33, at least seven of the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; or the amino acid residue at position 482 is different from a proline (P) residue. In still a further embodiment, the polypeptides comprising or consisting essentially of the amino acid sequence of SEQ ID NO: 33, the following amino acid residue substitutions are present: the amino acid residue at position 19 is different from a methionine (M) residue; the amino acid residue at position 22 is different from an arginine (R) residue; the amino acid residue at position 33 is different from a glycine (G) residue; the amino acid residue at position 173 is different from an arginine (R) residue; the amino acid residue at position 284 is different from an alanine (A) residue; the amino acid residue at position 313 is different from a proline (P) residue; the amino acid residue at position 468 is different from a glutamic acid (E) residue; and the amino acid residue at position 482 is different from a proline (P) residue.

In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least one of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least two of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least three of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least four of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least five of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least six of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least seven of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least eight of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least nine of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least 10 of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least 11 of any of the amino acid residue at positions 11, 19, 129, 136, 173, 269, 287, 314, 327, 376, 384, 424 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein the amino acid residue at positions 11, 19, 129, 136, 173, 269, 287, 314, 327, 376, 384, 424 and 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In an embodiment, the polypeptides of the present disclosure include, at position 11, an amino acid residue with a hydrophobic side chain (e.g., A, I, L, M, F, Y or W); and in yet another embodiment, a L residue. In an embodiment, the polypeptides of the present disclosure include, at position 19, an amino acid residue with a hydrophobic side chain (e.g., A, V, I, L, F, Y or W); and in yet another embodiment, a I residue. In an embodiment, the polypeptides of the present disclosure include, at position 129, an amino acid residue with a hydrophobic side chain (e.g., A, V, I, L, M, Y or W); and in yet another embodiment, a Y residue. In another embodiment, the polypeptides of the present disclosure include, at position 136, an amino acid residue with a hydrophobic side chain (e.g., A, V, I, M, F, Y or W); and in yet another embodiment, a F residue. In an embodiment, the polypeptides of the present disclosure include, at position 173, an amino acid residue with a hydrophobic side chain (e.g., A, V, I, L, M, F, Y or W); and in yet another embodiment, a I residue. In an embodiment, the polypeptides of the present disclosure include, at position 269, an amino acid residue with a hydrophobic side chain (e.g., A, V, I, L, M, F, Y or W); and in yet another embodiment, a L residue. In an embodiment, the polypeptides of the present disclosure include, at position 287, an amino acid residue with a hydrophobic side chain (e.g., V, I, L, M, F, Y or W); and in yet another embodiment, is a V residue. In an embodiment, the polypeptides of the present disclosure include, at position 314, an amino acid residue with a hydrophobic side chain (e.g., A, V, I, L, F, Y or W); in yet another embodiment, is a I or L residue; and in yet another embodiment, is a I residue. In an embodiment, the polypeptides of the present disclosure include, at position 327, an amino acid residue with a hydrophobic side chain (e.g., A, V, I, L, M, F, Y or W); in yet another embodiment, is a L residue. In an embodiment, the polypeptides of the present disclosure include, at position 376, an amino acid residue with a hydrophobic side chain (e.g., V, I, L, M, F, Y or W); in yet another embodiment, is a V residue. In an embodiment, the polypeptides of the present disclosure include, at position 384, an amino acid residue with a hydrophobic side chain (e.g., A, V, I, L, M, F, Y or W); and in yet another embodiment, is a I residue. In an embodiment, the polypeptides of the present disclosure include, at position 424, an amino acid residue with a hydrophobic side chain (e.g., A, V, I, L, M, F, Y or W); and in yet another embodiment, a L residue. In an embodiment, the polypeptides of the present disclosure include, at position 487, an amino acid residue with a hydrophobic side chain (e.g., A, V, I, L, M, F, Y or W); and in yet another embodiment, is a V residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least one of the amino acid residue at positions 22, 265, 374 and/or 468 comprises a positively charged side chain (Arginine, Histidine or Lysine, noted R, H and K, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least two of any of the amino acid residue at positions 22, 265, 374 and/or 468 comprises a positively charged side chain (Arginine, Histidine or Lysine, noted R, H and K, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least three of any of the amino acid residue at positions 22, 265, 374 and/or 468 comprises a positively charged side chain (Arginine, Histidine or Lysine, noted R, H and K, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, in which amino acid residue at positions 22, 265, 374 and 468 comprises a positively charged side chain (Arginine, Histidine or Lysine, noted R, H and K, respectively). In an embodiment, the polypeptides of the present disclosure include, at position 22, an amino acid residue with a positively charged side chain (e.g., K or H); and in yet another embodiment, a K residue. In an embodiment, the polypeptides of the present disclosure include, at position 265, an amino acid residue with a positively charged side chain (e.g., K or H); and in yet another embodiment, a K residue. In an embodiment, the polypeptides of the present disclosure include, at position 374, an amino acid residue with a positively charged side chain (e.g., R, K or H); and in yet another embodiment, a H residue. In an embodiment, the polypeptides of the present disclosure include, at position 468, an amino acid residue with a positively charged side chain (e.g., R, K or H); and in yet another embodiment, a K residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least one of the amino acid residue at positions 33, 87, 284 and/or 389 comprises a negatively charged side chain (Aspartic acid or Glutamic acid, noted D and E, respectively); In an embodiment, the polypeptides of the present disclosure include, at position 33, an amino acid residue with a negatively charged side chain (e.g., D or E); and in yet another embodiment, a D residue. In an embodiment, the polypeptides of the present disclosure include, at position 87, an amino acid residue with a negatively charged side chain (e.g., D or E); and in yet another embodiment, a D residue. In an embodiment, the polypeptides of the present disclosure include, at position 284, an amino acid residue with a negatively charged side chain (e.g., D or E); and in yet another embodiment, a D residue. In an embodiment, the polypeptides of the present disclosure include, at position 389, an amino acid residue with a negatively charged side chain (e.g., D or E); and in yet another embodiment, a D residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 1, wherein at least one of the amino acid residue at positions 41, 313 and/or 482 comprises a polar-uncharged side chain (Serine, Threonine, Asparagine or Glutamine, noted S, T, N and Q, respectively); In an embodiment, the polypeptides of the present disclosure include, at position 41, an amino acid residue with a polar-uncharged side chain (e.g., S, T, N or Q); and in yet another embodiment, a Q residue. In an embodiment, the polypeptides of the present disclosure include, at position 313, an amino acid residue with a polar-uncharged side chain (e.g., S, T, N or Q); and in yet another embodiment, a S residue. In an embodiment, the polypeptides of the present disclosure include, at position 482, an amino acid residue with a polar-uncharged side chain (e.g., S, T, N or Q); and in yet another embodiment, a S residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein at least one of the amino acid residue at positions 19, 136, 269, 314, 327, 376, 384, and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In an embodiment, the polypeptides of the present disclosure include, at position 19, an amino acid residue with a hydrophobic side chain (e.g., A, V, I, L, F, Y or W); and in yet another embodiment, a I residue. In another embodiment, the polypeptides of the present disclosure include, at position 136, an amino acid residue with a hydrophobic side chain (e.g., A, V, I, M, F, Y or W); and in yet another embodiment, a F residue. In an embodiment, the polypeptides of the present disclosure include, at position 269, an amino acid residue with a hydrophobic side chain (e.g., A, V, I, L, M, F, Y or W); and in yet another embodiment, a L residue. In an embodiment, the polypeptides of the present disclosure include, at position 314, an amino acid residue with a hydrophobic side chain (e.g., A, V, I, L, F, Y or W); in yet another embodiment, is a I or L residue; and in yet another embodiment, is a I residue. In an embodiment, the polypeptides of the present disclosure include, at position 327, an amino acid residue with a hydrophobic side chain (e.g., A, V, I, L, M, F, Y or W); in yet another embodiment, is a L residue. In an embodiment, the polypeptides of the present disclosure include, at position 376, an amino acid residue with a hydrophobic side chain (e.g., V, I, L, M, F, Y or W); in yet another embodiment, is a V residue. In an embodiment, the polypeptides of the present disclosure include, at position 384, an amino acid residue with a hydrophobic side chain (e.g., A, V, I, L, M, F, Y or W); and in yet another embodiment, is a I residue. In an embodiment, the polypeptides of the present disclosure include, at position 487, an amino acid residue with a hydrophobic side chain (e.g., A, V, I, L, M, F, Y or W); and in yet another embodiment, is a V residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein at least one of the amino acid residue at positions 265, 374 and/or 468 comprises a positively charged side chain (Arginine, Histidine or Lysine, noted R, H and K, respectively); In an embodiment, the polypeptides of the present disclosure include, at position 265, an amino acid residue with a positively charged side chain (e.g., K or H); and in yet another embodiment, a K residue. In an embodiment, the polypeptides of the present disclosure include, at position 374, an amino acid residue with a positively charged side chain (e.g., R, K or H); and in yet another embodiment, a H residue. In an embodiment, the polypeptides of the present disclosure include, at position 468, an amino acid residue with a positively charged side chain (e.g., R, K or H); and in yet another embodiment, a K residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein at least one of the amino acid residue at positions 33, 87, 284 and/or 389 comprises a negatively charged side chain (Aspartic acid or Glutamic acid, noted D and E, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein at least two of any one of the amino acid residue at positions 33, 87, 284 and/or 389 comprises a negatively charged side chain (Aspartic acid or Glutamic acid, noted D and E, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein at least three of any one of the amino acid residue at positions 33, 87, 284 and/or 389 comprises a negatively charged side chain (Aspartic acid or Glutamic acid, noted D and E, respectively). In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, the amino acid residue at positions 33, 87, 284 and 389 comprises a negatively charged side chain (Aspartic acid or Glutamic acid, noted D and E, respectively). In an embodiment, the polypeptides of the present disclosure include, at position 33, an amino acid residue with a negatively charged side chain (e.g., D or E); and in yet another embodiment, a D residue. In an embodiment, the polypeptides of the present disclosure include, at position 87, an amino acid residue with a negatively charged side chain (e.g., D or E); and in yet another embodiment, a D residue. In an embodiment, the polypeptides of the present disclosure include, at position 284, an amino acid residue with a negatively charged side chain (e.g., D or E); and in yet another embodiment, a D residue. In an embodiment, the polypeptides of the present disclosure include, at position 389, an amino acid residue with a negatively charged side chain (e.g., D or E); and in yet another embodiment, a D residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 2, wherein the amino acid residue at position 482 comprises a polar-uncharged side chain (Serine, Threonine, Asparagine or Glutamine, noted S, T, N and Q, respectively); In an embodiment, the polypeptides of the present disclosure include, at position 482, an amino acid residue with a polar-uncharged side chain (e.g., S, T, N or Q); and in yet another embodiment, a S residue.

In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 33, wherein at least one of the amino acid residue at positions 19, 173 and/or 487 when present, comprises a hydrophobic side chain (Alanine, Valine, Isoleucine, Leucine, Methionine, Phenylalanine, Tyrosine or Tryptophan, noted A, V, I, L, M, F, Y and W, respectively). In an embodiment, the polypeptides of the present disclosure include, at position 19, an amino acid residue with a hydrophobic side chain (e.g., A, V, I, L, F, Y or W); and in yet another embodiment, a I residue. In an embodiment, the polypeptides of the present disclosure include, at position 173, an amino acid residue with a hydrophobic side chain (e.g., A, V, I, L, M, F, Y or W); and in yet another embodiment, a I residue. In an embodiment, the polypeptides of the present disclosure include, at position 487, an amino acid residue with a hydrophobic side chain (e.g., A, V, I, L, M, F, Y or W); and in yet another embodiment, is a V residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 33, wherein at least one of the amino acid residue at positions 22 and/or 468 comprises a positively charged side chain (Arginine, Histidine or Lysine, noted R, H and K, respectively); In an embodiment, the polypeptides of the present disclosure include, at position 22, an amino acid residue with a positively charged side chain (e.g., K or H); and in yet another embodiment, a K residue. In an embodiment, the polypeptides of the present disclosure include, at position 468, an amino acid residue with a positively charged side chain (e.g., R, K or H); and in yet another embodiment, a K residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 33, wherein at least one of the amino acid residue at positions 33 and/or 284 comprises a negatively charged side chain (Aspartic acid or Glutamic acid, noted D and E, respectively); In an embodiment, the polypeptides of the present disclosure include, at position 33, an amino acid residue with a negatively charged side chain (e.g., D or E); and in yet another embodiment, a D residue. In an embodiment, the polypeptides of the present disclosure include, at position 284, an amino acid residue with a negatively charged side chain (e.g., D or E); and in yet another embodiment, a D residue. In another embodiment, the polypeptides exhibiting fumonisin esterase activity which is increased when compared to the control polypeptide comprising the amino acid of SEQ ID NO: 3 comprise or consist essentially of the amino acid sequence of SEQ ID NO: 33, wherein at least one of the amino acid residue at positions 313 and/or 482 comprises a polar-uncharged side chain (Serine, Threonine, Asparagine or Glutamine, noted S, T, N and Q, respectively); In an embodiment, the polypeptides of the present disclosure include, at position 313, an amino acid residue with a polar-uncharged side chain (e.g., S, T, N or Q); and in yet another embodiment, a S residue. In an embodiment, the polypeptides of the present disclosure include, at position 482, an amino acid residue with a polar-uncharged side chain (e.g., S, T, N or Q); and in yet another embodiment, a S residue.

In a specific embodiment, the polypeptides having esterase activity which is increased when compared to the control polypeptide comprising the amino acid of SEQ ID NO: 3 comprise or consist essentially of the amino acid sequence of SEQ ID NO: 6 to 32. In an embodiment, the polypeptides of the present disclosure comprises or consists essentially of the amino acid sequence of SEQ ID NO: 6. In an embodiment, the polypeptides of the present disclosure comprises or consists essentially of the amino acid sequence of SEQ ID NO: 7. In an embodiment, the polypeptides of the present disclosure comprises or consists essentially of the amino acid sequence of SEQ ID NO: 8. In an embodiment, the polypeptides of the present disclosure comprises or consists essentially of the amino acid sequence of SEQ ID NO: 9. In an embodiment, the polypeptides of the present disclosure comprises or consists essentially of the amino acid sequence of SEQ ID NO: 10. In an embodiment, the polypeptides of the present disclosure comprises or consists essentially of the amino acid sequence of SEQ ID NO: 11. In an embodiment, the polypeptides of the present disclosure comprises or consists essentially of the amino acid sequence of SEQ ID NO: 12. In an embodiment, the polypeptides of the present disclosure comprises or consists essentially of the amino acid sequence of SEQ ID NO: 13. In an embodiment, the polypeptides of the present disclosure comprises or consists essentially of the amino acid sequence of SEQ ID NO: 14. In an embodiment, the polypeptides of the present disclosure comprises or consists essentially of the amino acid sequence of SEQ ID NO: 15. In an embodiment, the polypeptides of the present disclosure comprises or consists essentially of the amino acid sequence of SEQ ID NO: 16. In an embodiment, the polypeptides of the present disclosure comprises or consists essentially of the amino acid sequence of SEQ ID NO: 17. In an embodiment, the polypeptides of the present disclosure comprises or consists essentially of the amino acid sequence of SEQ ID NO: 18. In an embodiment, the polypeptides of the present disclosure comprises or consists essentially of the amino acid sequence of SEQ ID NO: 19. In an embodiment, the polypeptides of the present disclosure comprises or consists essentially of the amino acid sequence of SEQ ID NO: 20. In an embodiment, the polypeptides of the present disclosure comprises or consists essentially of the amino acid sequence of SEQ ID NO: 21. In an embodiment, the polypeptides of the present disclosure comprises or consists essentially of the amino acid sequence of SEQ ID NO: 22. In an embodiment, the polypeptides of the present disclosure comprises or consists essentially of the amino acid sequence of SEQ ID NO: 23. In an embodiment, the polypeptides of the present disclosure comprises or consists essentially of the amino acid sequence of SEQ ID NO: 24. In an embodiment, the polypeptides of the present disclosure comprises or consists essentially of the amino acid sequence of SEQ ID NO: 25. In an embodiment, the polypeptides of the present disclosure comprises or consists essentially of the amino acid sequence of SEQ ID NO: 26. In an embodiment, the polypeptides of the present disclosure comprises or consists essentially of the amino acid sequence of SEQ ID NO: 27. In an embodiment, the polypeptides of the present disclosure comprises or consists essentially of the amino acid sequence of SEQ ID NO: 28. In an embodiment, the polypeptides of the present disclosure comprises or consists essentially of the amino acid sequence of SEQ ID NO: 29. In an embodiment, the polypeptides of the present disclosure comprises or consists essentially of the amino acid sequence of SEQ ID NO: 30. In an embodiment, the polypeptides of the present disclosure comprises or consists essentially of the amino acid sequence of SEQ ID NO: 31. In an embodiment, the polypeptides of the present disclosure comprises or consists essentially of the amino acid sequence of SEQ ID NO: 32. In an embodiment, the polypeptides of the present disclosure comprises or consists essentially of the amino acid sequence of SEQ ID NO: 40.

### Polypeptides Activity

In an embodiment, the polypeptide variants or fragments of the present disclosure have increased fumonisin esterase activity, when compared to the parent polypeptide. The term “activity” as used herein, refers to the number of moles of substrate converted to product by an enzyme preparation per unit time under specific conditions. In the context of the present disclosure, the polypeptides having fumonisin esterase activity convert fumonisin bearing at least one tricarballylic acids (TCA) moiety into their less toxic counterparts, namely partially hydrolyzed or hydrolyzed fumonisins. Fumonisin esterase activity is expressed as enzyme units (U)/mg of protein or as enzyme units (U)/mL of enzyme solution. The enzyme unit is defined as umol of TCA released per min (umol/min) in 20 mM Tris pH 8 with 100 uM fumonisin. In the context of this disclosure, the measured fumonisin esterase activity of the polypeptides can relate to i) the rate of secretion of the enzyme from the host cell and/or ii) its stability in various pH ranges and temperatures. Thus, the higher the rate of secretion of the enzyme, the more substrate it can convert and the more active it is. Similarly, the more stable the enzyme is (i.e., over a wide range of pH or temperature), the more substrate it will be able to convert in given reactive conditions and the more active it will be considered. In an embodiment, the polypeptide variants or fragments of the present disclosure have increased fumonisin esterase activity, when compared to the parent polypeptide of SEQ ID NO: 3. In another embodiment, the polypeptide variants or fragments of the present disclosure have increased fumonisin esterase secretion rate and/or increased stability, when compared to the parent polypeptide of SEQ ID NO: 3.

In an embodiment, the polypeptide variants or fragments of the present disclosure have increased fumonisin esterase secretion rate, when compared to the control polypeptide of SEQ ID NO: 3. In one embodiment, the secretion rate of the polypeptides is at least as high as of the parental one of SEQ ID NO: 3, when measured in same conditions. In one another embodiment, the secretion rate of the polypeptides is increased by at least 1.5, at least 2, at least 2.5, at least 3, at least 3.5, at last 4, at least 4.5, at least 5, at least 5.5, at least 6 or at least 6.5 times compared to the control polypeptide of SEQ ID NO: 3.

In an embodiment, the polypeptides (including associated variants or fragments) of the present disclosure have higher and/or more stable fumonisin esterase activity, when compared to the parent polypeptide. In another embodiment, the polypeptide variants or fragments of the present disclosure have higher residual fumonisin esterase activity after having been submitted to a heat challenge (e.g., higher thermostability), when compared to the parent polypeptide. In the context of the present disclosure, the term “thermostability” refers to the measured activity of the polypeptide variant, fragment or parent polypeptide when the polypeptide variant, fragment or parent polypeptide while it is being exposed and/or after it has been exposed to thermal challenge, also referred as heat shock in the Examples. In one embodiment, the residual fumonisin esterase activity is measured after a heat challenge performed at a temperature of at least 37° C., at least 40° C., at least 45° C., at least 46° C., at least 47° C., at least 48° C., at least 49° C., at least 50° C., at least 51° C. at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., or at least 70° C. or more. In another embodiment, the residual fumonisin esterase activity is measured after a heat challenge lasting at least 1, 2, 3, 4, 5, 6, 7, 8, 9, 10 minute or more. In one other embodiment, the residual activity of the polypeptides of the present disclosure is increased by at least 1.5, at least 2, at least 2.5, at least 3, or at least 3.5 times, when compared to the residual activity to the control polypeptide comprising the amino acid of SEQ ID NO: 3. In still another embodiment, the residual activity of the polypeptides of the present disclosure is increased by at least 10, 15, 20, 25, 30, 35, 40, 45, 50, 55, 60, 65, 70, 75, 80, 90% or more, when compared to the residual activity to the control polypeptide comprising the amino acid of SEQ ID NO: 3. In still another embodiment, the thermostability of the polypeptides of the present disclosure is increased by at least 1, 2, 3, 4, 5, 6, 7, 8, 9, 10, 11, 12, 13, 14, 15, 16, 17, 18, 19, 20° C. or more, when compared to the residual activity to the control polypeptide comprising the amino acid of SEQ ID NO: 3. As it is known in the art, thermostability can be measured by determining the T50 (e.g., the temperature at which 50% of the activity is lost) and/or the Tm (e.g., the melting point of the polypeptide or the temperature at which the polypeptide denatures). In a specific embodiment, the T50 of the polypeptides of the present disclosure is increased by at least 1, 2, 3, 4, 5, 6, 7, 8, 9, 10, 11, 12, 13, 14, 15, 16, 17, 18, 19, 20° C. or more, when compared to the T50 of the control polypeptide comprising the amino acid of SEQ ID NO: 3. In still another embodiment, the Tm of the polypeptides of the present disclosure is increased by at least 1, 2, 3, 4, 5, 6, 7, 8, 9, 10, 11, 12, 13, 14, 15, 16, 17, 18, 19, 20° C. or more, when compared to the Tm of the control polypeptide comprising the amino acid of SEQ ID NO: 3.

### Recombinant Host Cells

The polypeptides described herein can independently be provided in an isolated, synthetic or recombinant form (derived from the recombinant microbial host cell described herein) or derived from a recombinant microbial host cell expressing the polypeptides having fumonisin esterase activity. The recombinant microbial cell thus includes at least one genetic modification. In the context of the present disclosure, when recombinant microbial cell is qualified as “having a genetic modification” or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and are the results of in vitro manipulations of the recombinant host cell. When the genetic modification is the addition of a heterologous nucleic acid molecule, such addition can be made once or multiple times at the same or different integration sites. When the genetic modification is the modification of an endogenous nucleic acid molecule, it can be made in one or more copies of the targeted gene.

When expressed in a recombinant microbial host cell, the heterologous polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant microbial host cell. “Heterologous” also includes a native coding region, or portion thereof, that is introduced into the source organism in a form that is different from the corresponding native gene, e.g., not in its natural location in the organism's genome. The heterologous nucleic acid molecule is purposively introduced into the recombinant host cell. Thus, for example, an heterologous element could be derived from a different strain of host cell, or from an organism of a different taxonomic group (e.g., different domain, kingdom, phylum, class, order, family, genus, or species, or any subgroup within one of these classifications).

When a heterologous nucleic acid molecule is present in the recombinant microbial host cell, it can be integrated in the host cell's genome. The term “integrated” as used herein refers to genetic elements that are placed, through molecular biology techniques, into the genome of a microbial host cell. For example, genetic elements can be placed into the chromosomes of the microbial host cell as opposed to in a vector such as a plasmid carried by the host cell. Methods for integrating genetic elements into the genome of a host cell are well known in the art and include homologous recombination. The heterologous nucleic acid molecule can be present in one or more copies in the microbial host cell's genome. For example, the heterologous nucleic acid molecule can be present in 1, 2, 3, 4, 5, 6, 7, 8 or more copies in the microbial host cell's genome. Alternatively, the heterologous nucleic acid molecule can be independently replicating from the microbes' genome. In such embodiment, the nucleic acid molecule can be stable and self-replicating.

In the context of the present disclosure, a “microbial host cell” can be a bacterial host cell, a yeast host cell or a fungal host cell. The term “microbial host cell” necessarily excludes animal (including mammalian) and insect cells.

In the context of the present disclosure, the recombinant host cell can be a recombinant fungal cell, such as, for example, a recombinant yeast host cell or a recombinant mold host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Hanseniaspora, Kloeckera, Metschnikowia, Schwanniomyces, Wickerhamomyces or Yarrowia. Suitable yeast species can include, for example, S. cerevisiae, S. bulderi, S. barnetti, S. exiguus, S. uvarum, S. diastaticus, S. boulardii, K. lactis, K. marxianus or K. fragilis. In some embodiments, the recombinant yeast host cell is selected from the group consisting of Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some additional embodiments, the recombinant yeast host cell is from Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe, Metschnikowia sinensis, Metschnikowia fructicola, Metschnikowia pulcherima, Metschnikowia zobelli, Metschnikowia shanxiensis, Wickerhamomyces anomalus, Hanseniaspora guilliermondii, Hanseniaspora pseudoguilliermondii and/or Schwanniomyces occidentalis. In some embodiments, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidium, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae. In an embodiment, the recombinant yeast host cell is from the genus Pichia and, in some embodiments, from the species Pichia pastoris.

Suitable fungal host cell can be, for example, from the genus Aspergillus or Trichoderma.

The microbial host cell can be a bacterial host cell. Suitable bacterial host cells that can be genetically modified as described herein can be a Gram-positive or a Gram-negative bacteria. The recombinant bacterial host cell can be, for example, from the phylum Acidobacteria, Actinobacteria, Aquificae, Bacillus, Bacteroidetes, Chlamydiae, Cholorobi, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermotogae or Verrucomicrobia. In some embodiments, the bacterial host cell is from one of the following genus Acidobacterium, Geothrix, Holophaga, Acidimicrobium. Kribella, Atopobium, Collinsella, Coriobacterium, Cryptobacterium, Denitrobacterium, Eggerthella, Slackia, Rubrobacter, Sphaerobacter, Aquifex, Hydrogenivirga, Hydrogenobacter, Hydrogenobaculum, Thermocrinis, Hydrogenothermus, Persephonella, Sulfurihydrogenibium, Venenivibrio, Bacteroides, Acetofilamentum, Acetomicrobium, Acetothermus, Anaerorhabdus, Megamonas, Rikenella, Marinilabilia, Porphyromonas, Dysgonomonas, Prevotella, Chlamydia, Chlamydophila, Simkania, Fritschea, Simkania, Fritschea, Chrysiogenes, Deferribacter, Denitrovibrio, Flexistipes, Geovibrio, Deinococcus, Thermus, Meiothermus, Marinithermus, Oceanithermus, Vulcanithermus, Dictyoglomus, Hepatoplasma, Mycoplasma, Ureaplasma, Entomoplasma, Mesoplasma, Spiroplasma, Anaeroplasma, Asteroleplasma, Erysipelothrix, Holdemania, Acholeplasma, Phytoplasma, Fusobacterium, Gemmatimonas, Nitrospira, Gemmata, Isosphaera, Pirellula, Planctomyces, Brocadia, Kuenenia, Scalindua, Anammoxoglobus, Jettenia, Asticcacaulis, Brevundimonas, Caulobacter, Phenylobacterium, Kordiimonas, Parvularcula, Aurantimonas, Fulvimarina, Bartonella, Beijerinckia, Chelatococcus, Derxia, Methylocella, Afipia, Agromonas, Blastobacter, Bosea, Bradyrhizobium, Nitrobacter, Oligotropha, Photorhizobium, Rhodoblastus, Rhodopseudomonas, Brucella, Mycoplana, Ochrobactrum, Ancalomicrobium, Ancylobacter, Angulomicrobium, Aquabacter, Azorhizobium, Blastochloris, Devosia, Dichotomicrobium, Filomicrobium, Gemmiger, Hyphomicrobium, Labrys, Methylorhabdus, Pedomicrobium, Prosthecomicrobium, Rhodomicrobium, Rhodoplanes, Seliberia, Starkeya, Xanthobacter, Methylobacterium, Microvirga, Protomonas, Roseomonas, Methylocystis, Methylosinus, Methylopila, Aminobacter, Aquamicrobium, Defluvibacter, Hoeflea, Mesorhizobium, Nitratireductor, Parvibaculum, Phyllobacterium, Pseudaminobacter, Agrobacterium, Rhizobium, Sinorhizobium, Liberibacter, Ahrensia, Albidovulum, Amaricoccus, Antarctobacter, Catellibacterium, Citreicella, Dinoroseobacter, Haematobacter, Jannaschia, Ketogulonicigenium, Leisingera, Loktanella, Maribius, Marinosulfonomonas, Marinovum, Maritimibacter, Methylarcula, Nereida, Oceanibulbus, Oceanicola, Octadecabacter, Palleronia, Pannonibacter, Paracoccus, Phaeobacter, Pseudorhodobacter, Pseudovibrio, Rhodobaca, Rhodobacter, Rhodothalassium, Rhodovulum, Roseibacterium, Roseibium, Roseicyclus, Roseinatronobacter, Roseisalinus, Roseivivax, Roseobacter, Roseovarius, Rubrimonas, Ruegeria, Sagittula, Salipiger, Silicibacter, Staleya, Stappia, Sulfitobacter, Tetracoccus, Thalassobacter, Thalassobius, Thioclava, Yangia, Azospirillum, Dechlorospirillum, Defluvicoccus, Inquilinus, Magnetospirillum, Phaeospirillum, Rhodocista, Rhodospira, Rhodospirillum, Rhodovibrio, Roseospira, Skermanella, Thalassospira, Tistrella, Acetobacter, Acidicaldus, Acidiphilium, Acidisphaera, Acidocella, Acidomonas, Asaia, Belnapia, Craurococcus, Gluconacetobacter, Gluconobacter, Kozakia, Leahibacter, Muricoccus, Neoasaia, Oleomonas, Paracraurococcus, Rhodopila, Roseococcus, Rubritepida, Saccharibacter, Stella, Swaminathania, Teichococcus, Zavarzinia, Rickettsia, Orientia, Wolbachia, Aegyptianella, Anaplasma, Cowdria, Ehrlichia, Neorickettsia, Caedibacter, Holospora, Lyticum, Odyssella, Symbiotes, Tectibacter, Blastomonas, Citromicrobium, Erythrobacter, Erythromicrobium, Kaistobacter, Lutibacterium, Novosphingobium, Porphyrobacter, Sandaracinobacter, Sphingobium, Sphingomonas, Sphingopyxis, Zymomonas, Achromobacter, Alcaligenes, Bordetella, Pelistega, Sutterella, Taylorella, Burkholderia, Chitinimonas, Cupriavidus, Lautropia, Limnobacter, Pandoraea, Paucimonas, Polynucleobacter, Ralstonia, Thermothrix, Acidovorax, Aquabacterium, Brachymonas, Comamonas, Curvibacter, Delftia, Hydrogenophaga, Ideonella, Leptothrix, Limnohabitans, Pelomonas, Polaromonas, Rhodoferax, Roseateles, Sphaerotilus, Tepidimonas, Thiomonas, Variovorax, Collimonas, Duganella, Herbaspirillum, Herminiimonas, Janthinospirillum, Massilia, Naxibacter, Oxalobacter, Oxalicibacterium, Telluria, Borrelia, Brevinema, Cristispira, Spirochaeta, Spironema, Treponema, Brachyspira (Serpulina), Leptospira, Leptonema, Thermodesulfobacterium, Thermotoga, Verrucomicrobium, Prosthecobacter, Pediococcus, and Akkermansia. In one particular embodiment, the recombinant bacterial host cell is from the genus Escherichia and, in some additional embodiments, from the species Escherichia coli. In one particular embodiment, the recombinant bacterial host cell is from the genus Bacillus and, in some additional embodiments, from the species Bacillus subtilis. In one specific embodiment, the recombinant bacterial host cell is from the genus Lactobacillus, and in some additional embodiments, from the species Lacticaseibacillus casei (previously known as Lactobacillus casei).

Polypeptides having the fumonisin esterase activity are expressed from one or more heterologous nucleic acid molecules in one or more recombinant microbial host cell. As such, the polypeptide having fumonisin esterase activity are heterologous with respect to the recombinant microbial host cell expressing them. As used herein, the term “heterologous” when used in reference to a nucleic acid molecule (such as a promoter, a terminator or a coding sequence) or a polypeptide refers to a nucleic acid molecule or a polypeptide that is not natively found in the recombinant host cell. “Heterologous” also includes a native coding region/promoter/terminator, or portion thereof, that was introduced into the source organism in a form and/or at a location that is different from the corresponding native gene, e.g., not in its natural location in the organism's genome. The heterologous nucleic acid molecule is purposively introduced into the recombinant microbial host cell. For example, a heterologous element could be derived from a different strain of host cell, or from an organism of a different taxonomic group (e.g., different domain, kingdom, phylum, class, order, family, genus, or species, or any subgroup within one of these classifications).

In some embodiments, the recombinant microbial host cell comprises a genetic modification (e.g., a heterologous nucleic acid molecule) allowing the recombinant expression of the polypeptide having fumonisin esterase activity. In such embodiment, a heterologous nucleic acid molecule encoding the polypeptide having fumonisin esterase activity can be introduced in the microbial host cell to express the polypeptide having fumonisin esterase activity. The expression of the polypeptide having fumonisin esterase activity can be constitutive or induced (for example, by the supplementation of the culture medium with an inducing agent, for example, IPTG). The expression of the polypeptide having fumonisin esterase activity can occur during the propagation phase or aerobic growth of the recombinant microbial host cell and/or the fermentation phase or any other anaerobic growth of the recombinant microbial host cell.

The polypeptide of the present disclosure can be expressed inside the recombinant microbial host cell, e.g., intracellularly or intracellular form. The polypeptides of the present disclosure can be modified to remove, if any, signal peptide sequences present in the native amino acid sequence of the polypeptide to allow for an intracellular expression. In some embodiments, the polypeptides of the present disclosure can be modified to replace the signal sequence with a N-terminus modification to allow for an intracellular expression.

The polypeptide of the present disclosure can be exported and remain physically associated with the recombinant microbial host cell (e.g., a membrane-associated form). In an embodiment, at least one portion (usually at least one terminus) of the heterologous polypeptide is bound, covalently, non-covalently and/or electrostatically for example, to the cell wall (and in some embodiments to the cytoplasmic membrane) of the recombinant microbial host cell. For example, the heterologous polypeptide can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous polypeptide may be tethered and not be directly bound to the cell membrane or cell wall (e.g., such as when binding occurs via a tethering moiety), the protein is nonetheless considered a “cell-associated” heterologous polypeptide according to the present disclosure.

The polypeptide of the present disclosure can be expressed outside the recombinant microbial host cell, e.g., secreted form. The polypeptides of the present disclosure can be modified to add/replace a signal peptide sequences to allow or facilitate secretion. In some embodiments, the recombinant microbial host cell can be further genetically modified to favor the secretion of the polypeptides of the present disclosure. Such modifications include, but are not limited to, those described in U.S. provisional application 63/141,807 filed on Jan. 26, 2021 and herewith incorporated in its entirety.

### Nucleic Acid Molecules for Expressing the Heterologous Polypeptides Having Fumonisin Esterase Activity

In some embodiments, the nucleic acid molecules encoding the polypeptides, fragments or variants that can be introduced into the recombinant microbial host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleotide acid molecule coding for the polypeptide in a manner that allows, under certain conditions, for expression of the peptide from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous polypeptide. In the context of the present disclosure, one or more than one promoter can be included in the nucleic acid molecule. When more than one promoter is included in the nucleic acid molecule, each of the promoters is operatively linked to the nucleic acid sequence coding for the polypeptide. The promoters can be located, in view of the nucleic acid molecule coding for the polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same, or species of the yeast host cell and the polypeptide is derived from different genera that the host cell. One or more promoters can be used to allow the expression of the polypeptides in the recombinant yeast host cell.

In some embodiments, the host is a facultative anaerobe, such as S. cerevisiae. For facultative anaerobes, cells tend to propagate or ferment depending on the availability of oxygen. In a fermentation process, yeast cells are generally allowed to propagate before fermentation is conducted. In some embodiments, the promoter preferentially initiates transcription during a propagation phase such that the polypeptides are expressed during the propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (e.g., under anaerobic conditions) to maximize the production of one or more desired metabolites.

In some embodiments, the promoter or the combination of promoters present in the heterologous nucleic acid is capable of allowing the expression of the recombinant heterologous polypeptide during the propagation phase of the recombinant microbial host cell. This will allow the accumulation of the polypeptide associated with the recombinant microbial host cell prior to any subsequent use, for example in liquefaction or fermentation. In some embodiments, the promoter allows the expression of the polypeptide during the propagation phase.

In other embodiments, the promoter or the combination of promoters present in the heterologous nucleic acid is capable of allowing the expression of the recombinant heterologous polypeptide during the anaerobic growth or culture (for example, in the fermentation phase) of the recombinant microbial host cell.

The promoters that can be included in the heterologous nucleic acid molecule can be constitutive or inducible promoters. Inducible promoters include, but are not limited to glucose-regulated promoters (e.g., the promoter of the hxt7 gene (referred to as hxt7p), a functional variant or a functional fragment thereof; the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), molasses-regulated promoters (e.g., the promoter of the mol1 gene (referred to as mol1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the ctt1 gene (referred to as ctt1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof), nitrogen-regulated promoters (e.g., the promoter of the ygp1 gene (referred to as ygp1p), a functional variant or a functional fragment thereof), promoter of the adh1 gene (referred to as adh1p), a functional variant or a functional fragment thereof and the methanol-inducible promoter of the aox1 gene (referred to as aox1p), a functional variant or a functional fragment thereof.

Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof), of the stl1 gene (referred to as stl1p, a functional variant or a functional fragment thereof) and/or of the tef2 gen (referred to as tef2p, a functional variant or a functional fragment thereof).

Promoters that can be included in the heterologous nucleic acid molecule of the present disclosure include, without limitation, phage-derived promoters, such as the T5 or the T7 promoter. These promoters are particularly useful for the expression of the polypeptide having fumonisin esterase activity in a bacterial host cell, such as Escherichia coli.

In the context of the present disclosure, the expression “functional fragment of a promoter” refers to a shorter nucleic acid sequence than the native promoter which retain the ability to control the expression of the nucleic acid sequence encoding the heterologous polypeptides. Usually, functional fragments are either 5′ and/or 3′ truncation of one or more nucleic acid residue from the native promoter nucleic acid sequence.

In the context of the present disclosure, the expression “functional fragment of a promoter” refers to a nucleic acid sequence which differs in at least one position and still retain the ability to control the expression of the nucleic acid sequence encoding the heterologous polypeptide. In some embodiments, the heterologous nucleic acid molecules include one or a combination of terminator sequence(s) to end the translation of the heterologous protein (or of the chimeric protein comprising same). The terminator can be native or heterologous to the nucleic acid sequence encoding the heterologous protein or its corresponding chimera. In some embodiments, one or more terminators can be used. In some embodiments, the terminator comprises the terminator derived from is from the dit1 gene (dit1t, a functional variant or a functional fragment thereof), from the idp1 gene (idp1t, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), and/or from the ira2 gene (ira2t, a functional variant or a functional fragment thereof). In an embodiment, the terminator comprises or is derived from the dit1 gene (dit1t, a functional variant or a functional fragment thereof). In another embodiment, the terminator comprises or is derived adh3t and/or idp1t. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera. The heterologous nucleic acid molecules of the present disclosure can also include a portion encoding a signal sequence which is operatively linked to the portion encoding the heterologous polypeptide having fumonisin amine oxidase. The nucleic acid portion encoding the signal sequence is usually located 3′ to the promoter and 5′ to the portion encoding the heterologous polypeptide having fumonisin amine oxidase. The heterologous nucleic acid molecules, especially designed to be used in eukaryotic cells, can also include a 5′ untranslated region (UTR) between the one or more promoters and the heterologous polypeptide reading frame. In some embodiments, the 5′ UTR is associated with or derived from the one or more promoters used in the heterologous nucleic acid molecule.

### Compositions and Forms of Polypeptides, Variants and Fragments Having Fumonisin Esterase Activity

The polypeptides, variants, and fragments of the present disclosure are provided in a “recombinant form” because they have been produced by recombinant technology using genetic engineering to express the polypeptides, variants, and fragments in recombinant microbial host cells. As such, the present disclosure provides a composition comprising a recombinant microbial host cell capable of expressing the polypeptide, variant, or fragment of the present disclosure. In some embodiments, the composition further comprises, in addition to the recombinant microbial host cell, the polypeptide, variant, or fragment of the present disclosure. The recombinant microbial host can be a yeast host cell, a fungal host cell or a bacterial host cell. In an embodiment, the recombinant yeast host cell is from the genus Saccharomyces and, in some additional embodiments, from the species Saccharomyces cerevisiae. In an embodiment, the recombinant yeast host cell is from the genus Torulaspora and, in some additional embodiments, from the species Torulaspora delbrueckii. The recombinant yeast host cell can be from the genus Pichia and, in some additional embodiments, from the species Pichia pastoris. In some embodiments, when the composition comprises a recombinant yeast host cell, it can provided as a cream yeast. In alternative embodiments, when the composition comprises a recombinant yeast host cell, it can be provided in a dried form, including, for example, a roller dried form, a spray dried form, a fluid bed dried form or lyophilized form. The recombinant fungal host cell can be from the genus Aspergillus or Trichoderma. The recombinant bacterial host cell can be from the genus Bacillus, and in some additional embodiments, from the species Bacillus subtilis. The recombinant bacterial host cell can be from the genus Escherichia, and in some additional embodiments, from the species Escherichia coli. The recombinant bacterial host cell can be from the genus Pediococcus. The recombinant bacterial host cell can be from the genus Lacticaseibacillus and in some additional embodiments, from the species Lacticaseibacillus casei. In some embodiments, when the composition comprises the bacterial host cell, it is provided in a frozen, spray dried, freeze dried, fluid bed dried or lyophilized form.

The present disclosure provides polypeptides, variants and fragments having fumonisin esterase activity which can be provided in a semi-purified or in a substantially purified form. As used in the context of the present disclosure, the expression “semi-purified form” refers to the fact that the polypeptides, variants, and fragments have been physically dissociated, at least in part, from the components of the recombinant microbial host cell expressing same. The expression “substantially purified form” refers to the fact that the polypeptides, variants, and fragments have been physically dissociated from the majority of the components of the recombinant microbial host cell expressing same. In an embodiment, a composition comprising a polypeptide, variant or fragment in a substantially purified form is at least 90%, 95%, 96%, 97%, 98% or 99% pure. In some embodiments, the composition comprising a polypeptide, variant or fragment lacks a detectable amount of deoxyribonucleic acids from the microbial host cell used to express it.

The compositions of the present disclosure can include, besides the polypeptides, variants, or fragments, a microbial cell (living, inactivated or dead) or at least one component of a microbial cell. In an embodiment, the microbial cell is a recombinant microbial host cell capable of expressing and/or having expressed the polypeptides, variants, or fragments of the present disclosure. The “at least one component of a microbial cell” can be an intracellular component and/or a component associated with the microbial host cell's wall or membrane. The “at least one component of a microbial cell” can include a protein, a peptide or an amino acid, a carbohydrate and/or a lipid. The “at least one component of a microbial cell” can include a microbial host cell organelle. The “at least one component of a microbial cell” can be a microbial extract, such as, for example, a bacterial extract, a fungal extract or a yeast extract. The composition can be an inactive composition (e.g., none of the microbial cell are alive), a semi-active or inactivated composition (e.g., some of the microbial cells are alive) or an active composition (e.g., most of the microbial host cells are alive). The composition can be a liquid or a solid (e.g., dried, frozen and/or lyophilized) product. Inactivated yeast products include, but are not limited to a yeast extract. Active/semi-active yeast products include, but are not limited to, a cream yeast, an instant dried yeast or an active-dried yeast. Inactivated bacterial products, include but are not limited to a bacterial extract. An active/semi-active bacterial products include, but are not limited to, bacterial concentrates. Inactivated fungal products, include but are not limited to a fungal extract. An active/semi-active fungal products include, but are not limited to, fungal concentrates. In some embodiments, the yeast product is a yeast extract produced from recombinant yeast host cells expressing the polypeptides. In some additional embodiment, the bacterial product is a bacterial extract produced from the recombinant microbial host cells expressing the polypeptides. In some additional embodiment, the fungal product is a fungal extract produced from the recombinant microbial host cells expressing the polypeptides.

The composition of the present disclosure can include, in some embodiments, a binder. As used in the present disclosure, a “binder” is a component capable of physically associated (e.g., binding) to a mycotoxin (such as, for example, a fumonisin mycotoxin) to reduce its bioavailability. The binder can include a mineral binder, an organic binder or both. An exemplary mineral binder is a clay, such as a bentonite clay. An exemplary organic binder is a microbial component, such as, for example, a microbial extract (e.g., a bacterial extract, a yeast extract and/or a fungal extract) and/or an algal component.

In an embodiment, the composition of the present disclosure is provided as an additive intended to be combined with at least one food ingredient, at least one feed ingredient, and/or at least one beverage ingredient. The additive can be, in some embodiments, included directly in a food product, a feed product and/or a beverage product. In such embodiments, the polypeptides, variants, and fragments that may be present in the composition can be admixed with a further feed additive, a further food additive, a further beverage additive and/or a binder. When the composition of the present disclosure is intended to be used as an additive, it can be provided in a liquid form, which can be, in some further embodiments, a spray-dryable liquid form. Alternatively or in combination, the composition can be provided in a powder form, which can, in some further embodiments, be a free-flowing powder.

In some embodiments, the composition of the present disclosure can include one or more additional polypeptides capable of detoxifying a mycotoxin (such as a fumonisin mycotoxin). In some specific embodiments, the composition of the present disclosure can include two or more distinct polypeptides, variants, or fragments described herein. In some alternative specific embodiments, the composition of the present disclosure can include one or more polypeptide having fumonisin amine oxidase activity, such as, for example, those described in WO2020047656 which is herewith incorporated in its entirety.

### Methods of Using the Polypeptides Having Fumonisin Esterase Activity

The polypeptides having fumonisin esterase activity, associated variants and fragments as well as compositions comprising same can be used to detoxify a fumonisin mycotoxin. In the context of the present disclosure, the expression “detoxify a fumonisin mycotoxin” refers to the ability of polypeptides having fumonisin esterase activity to cause, under the appropriate conditions, the hydrolysis of one or more tricarballylic acid moiety from the fumonisin mycotoxin which, in return, reduces or eliminates its toxicity.

The methods of the present disclosure include a step of contacting a polypeptide having fumonisin esterase activity (variants, fragments and/or compositions comprising same as the control polypeptide having the amino acid sequence of SEQ ID NO: 3) with a fumonisin mycotoxin so as to cause the hydrolysis of at least one tricarballylic acid moiety from the fumonisin mycotoxin. The polypeptides of the present disclosure (variants, fragments and/or compositions comprising same) are contacted with the fumonisin mycotoxin in a “detoxifying amount” to cause, as indicated herein, the hydrolysis of at least one tricarballylic acid moiety from the fumonisin mycotoxin. The polypeptides of the present disclosure (variants, fragments and/or compositions comprising same) are contact with the fumonisin mycotoxin under temperature, pH, salt conditions allowing the hydrolysis of at least one tricarballylic acid acid moiety from the fumonisin mycotoxin. In some embodiments, the methods of the present disclosure can be used to convert at least 5, 10, 15, 20, 25, 30, 35, 40, 45, 50, 55, 60, 65, 70, 75, 80, 85, 90, 95% or more of the fumonisin mycotoxin into its hydrolyzed (less toxic) form. The fumonisin mycotoxin can be present in a plant or a plant-based material and/or an animal or animal-based material. As such, in some embodiments, the contacting step can occur between the polypeptides of the present disclosure (variants, fragments and/or compositions comprising same) and the plant or the plant-based material. As it is known in the art, fumonisin mycotoxins can be found, for example, in silage (maize (including corn), grass, sorghum, sweet potato vines for example), hay, straw, grains (maize (including corn), oat, wheat, rye, barley, rice for example), grain by-products (distillers grains for examples), legumes (peanut and soybean for example), cottonseed meal, vegetables (cabbage, carrots, corn for example), fruits, milk, milk by-products (whey for example), food products (corn flour, high-fructose corn syrup for example) as well as in animal feed products. The polypeptides of the present disclosure (variants, fragments and/or compositions comprising same) can be used to detoxify such fumonisin mycotoxin-containing materials. Materials suspected of being contaminated with fumonisin mycotoxins which could benefit from being detoxified with the polypeptides of the present disclosure include, without limitation, a food ingredient, a food product, a feed ingredient, a feed product, a beverage ingredient, a beverage product, a biomass intended to be fermented and/or a fermented biomass. As such, in some additional embodiments, the methods of the present disclosure can be used to make a food ingredient (e.g., a component included in a food product), a food product, a feed ingredient (e.g., a component included in a feed product), a feed product, a beverage ingredient (e.g., a component included in a beverage product), a beverage product, a biomass intended to be fermented and/or a fermented biomass. In some embodiments, the polypeptides of the present disclosure (variants, fragments and/or compositions comprising same) are used to detoxify a food ingredient prior to its introduction into a food product. Alternatively, or in combination, the polypeptides of the present disclosure (variants, fragments and/or compositions comprising same) are included in a food product, as a food additive, to detoxify the food product prior to consumption and/or to detoxify the food product during its absorption by a subject. In some embodiments, the polypeptides of the present disclosure (variants, fragments and/or compositions comprising same) are used to detoxify a feed ingredient prior to its introduction into a feed product. Alternatively, or in combination, the polypeptides of the present disclosure (variants, fragments and/or compositions comprising same) are included in a feed product, as a feed additive, to detoxify the feed product prior to consumption and/or to detoxify the feed product during its absorption by a subject. In some embodiments, the polypeptides of the present disclosure (variants, fragments and/or compositions comprising same) are used to detoxify a beverage ingredient prior to its introduction into a beverage product. Alternatively, or in combination, the polypeptides of the present disclosure (variants, fragments and/or compositions comprising same) are included in a beverage product, as a beverage additive, to detoxify the beverage product prior to consumption and/or to detoxify the beverage product during its absorption by a subject. In some embodiments, the polypeptides of the present disclosure (variants, fragments and/or compositions comprising same) are used to detoxify a biomass prior to, during and/or after its fermentation. In some embodiments, the detoxified and fermented biomass (or a component derived therefrom such as brewers' grand and/or distillers' grains) can be used as a feed ingredient, a feed additive and/or a feed product.

In the methods of the present disclosure, the contacting step can be conducted at a temperature higher than 4° C. and lower than 95° C. In an embodiment, the contacting step is conducted at a temperature of at least 5, 10, 15, 20, 25, 30, 35, 37 40, 45, 50, 55, 60, 65, 70, 75, 80, 85 or 90° C. In another embodiment, the contacting step is conducted at a temperature of no more than 90, 85, 80, 75, 70, 65, 60, 55, 50, 45, 40, 35, 30, 25, 20, 15, 10 or 5° C. In yet another embodiment, the contacting step is conducted at a temperature between 5, 10, 15, 20, 25, 30, 35, 40, 45, 50, 55, 60, 65, 70, 75, 80, 85 or 90° C. and 90, 85, 80, 75, 70, 65, 60, 55, 50, 45, 40, 35, 30, 25, 20, 15, 10 or 5° C. In a further embodiment, the contacting step is conducted at a temperature between 4 and 45° C. In a yet further embodiment, the contacting step is conducted at a temperature between 2° and 40° C., for example, at a temperature of 37° C.

In the methods of the present disclosure, the contacting step can be conducted at a certain pH or pH range. For example, the contacting step can be conducted at a pH of at least 2, 3, 4, 5, 6, 7, 8 or higher. In another example, the contacting step can be conducted at a pH of no more than 8, 7, 6, 5, 4, 3 or 2. In still another example, the contacting step can be conducted at a pH between 2 and 8.5, for example, between a pH of at least 2, 3, 4, 5, 6, 7 or 8 and a pH of no more than 8.5, 8 7, 6, 5, 4 or 3. In a specific example, the contacting step can be conducted at a pH between 5 and 7, for example, at a pH of 6. In another embodiment, the contacting step is conducted at a temperature of 37° C. and a pH of 6. The contacting can be done with directly with solid components. The contacting can be done between solid and liquid components. The contacting can be done directly with liquid components.

In an embodiment, the method includes a step of determining if the material is contaminated with the fumonisin mycotoxin either prior to and/or after the contact with the polypeptides of the present disclosure (variants, fragments and/or compositions comprising same).

The methods of the present disclosure include, in some embodiments, administering the polypeptides of the present disclosure (variants, fragments and/or compositions comprising same) directly or indirectly to a subject. In some embodiments, the subject can be a mammal, such as for example a human or an animal (such as a ruminant (including but not limited to, a cattle, a sheep, a goat, a buffalo, a giraffe, a deer, a moose, an elk, a yak, a bison, etc.), a swine (pig, piglet, sow, etc.), etc.). In some embodiments, the subject is an animal such as, for example a horse, a bird (including but not limited to a chicken, a goose, a duck, a fowl, a quail, a turkey, etc.), a fish or an aquatic animal (a shrimp for example).

As it is known in the art, silage is a material which is often contaminated with fumonisin mycotoxins. Such contamination typically occurs in the field prior to harvest and silage preparation. As such, in some embodiments, the polypeptides of the present disclosure (variants, fragments and/or compositions comprising same) can be contacted with silage to decrease and prevent the accumulation of the fumonisin mycotoxin during storage and prior to its administration to animals. In the latter embodiment, the polypeptides of the present disclosure (variants, fragments and/or compositions comprising same) can be used to detoxify silage prior to its administration to animals and/or administered to animals prior to, during and/or after they have consumed a potentially fumonisin mycotoxin-contaminated silage. In the latter embodiment, the detoxification step can occur, at least partially, in situ in the animals' gastro-intestinal tract.

Feed destined for animal consumption can also be contaminated with fumonisin mycotoxins. Such contamination can occur in the feed ingredient prior to its inclusion in the feed product (in the field and/or during silage preparation). As such, in some embodiments, the polypeptides of the present disclosure (variants, fragments and/or compositions comprising same) can be contacted with a feed ingredient and/or a feed product to decrease mycotoxin during storage and/or after storage and prior to its consumption by animals. In the latter embodiment, the polypeptides of the present disclosure (variants, fragments and/or compositions comprising same) can be used to detoxify feed ingredients and feed products prior to their consumption and/or administered to animals prior to, during and/or after they have consumed a potentially fumonisin mycotoxin-contaminated food product. In the latter embodiment, the detoxification step can occur, at least partially, in situ in the animal's gastro-intestinal tract. The methods of the present disclosure can also include crushing, grinding, sieving, filtering and/or pelleting prior to or after the detoxification step.

Silage is also susceptible to be contaminated with fumonisin mycotoxins. As such, the present disclosure comprises contacting silage prior to its administration to the animals with the polypeptides, variants, or fragments of the present disclosure. This contact can occur during the storage of silage. This contact can occur prior to administration to the animals to limit the toxicity of the fumonisin mycotoxins that such animals may consume. In some embodiments, a living recombinant microbial cell of the present disclosure is contacted with silage and expresses the polypeptides, the variants, or the fragments in situ to detoxify silage.

In some further embodiments, the recombinant microbial host cells of the present disclosure can be administered directly to subjects, such as humans or animals, to provide them with a source of the polypeptides, the variants, or the fragments of the present disclosure. In such embodiment, the recombinant microbial host cell may be derived from a probiotic microbial host cell, live microorganisms which when administered in adequate amounts confer a health benefit on the subjected. For example, probiotic microbial host cells were demonstrated to play a key role in restoring and maintaining intestinal flora balance, in supporting immune system but also in affording beneficial effects on the brain-gut axis and metabolic health. In an embodiment, the probiotic microbial host cells can be probiotic bacteria or probiotic yeasts. In some embodiments, the probiotic bacteria are, without being limited to, Lacticaseibacillus species, Pediococcus species, Bifidobacteria species, Lactococcus species, Streptococcus species, Enterococcus species, Escherichia species, Clostridium species, and Bacillus species. In another embodiment, the probiotic yeasts are, without being limited to, Saccharomyces species (i.e., S. cerevisiae, S. cerevisiae var boulardii, etc.), Candida species (i.e., Candida utilis, Candida guilliermondii, etc.), Pichia species (i.e., P. anomala, P. pastoris, etc.), Metschnikowia species (Metschnikowia sinensis, Metschnikowia fructicola, Metschnikowia pulcherima, Metschnikowia zobelli, Metschnikowia shanxiensis, etc.), Wickerhamomyces species (i.e., Wickerhamomyces anomalus) or Hanseniaspora species (i.e., Hanseniaspora guilliermondii, Hanseniaspora pseudoguilliermondii, etc.). In yet another embodiment, the probiotic yeast is S. cerevisiae L60, S. cerevisiae L62, S. cerevisiae L69 or S. cerevisiae L72. In yet another embodiment, the probiotic yeast is the S. cerevisiae var boulardii strain deposited under accession number I-1079 at the CNCM. In yet another embodiment, the probiotic yeast is C. utilis L75.

Food destined for human consumption can also be contaminated with fumonisin mycotoxins. Such contamination can occur in the food ingredient prior to its inclusion in the food product and/or in the stored food product during the storage of same. As such, in some embodiments, the polypeptides of the present disclosure (variants, fragments and/or compositions comprising same) can be contacted with a food ingredient and/or a food product to prevent the accumulation of the fumonisin mycotoxin during storage and/or after storage and prior to its consumption by humans. In the latter embodiment, the polypeptides of the present disclosure (variants, fragments and/or compositions comprising same) can be used to detoxify food ingredients and food products prior to their consumption and/or administered to humans prior to, during and/or after they have consumed a potentially fumonisin mycotoxin-contaminated food product. In the latter embodiment, the detoxification step can occur, at least partially, in situ in the human's gastro-intestinal tract. The methods of the present disclosure can also include crushing, grinding, sieving, filtering, baking, freezing and/or frying prior to or after the detoxification step. The food product can be derived from grains and can be, for example, a flour. The flour can be a corn flour, a wheat flour, a barley flour, a buckwheat flour, a chickpea flour, etc.

In some embodiments, the feed ingredient, the feed product, the food ingredient, the food product, the beverage ingredient and/or the beverage ingredient comprises between 1 to 99% of corn or a product derived from corn.

The methods of the present disclosure can include a step of fermenting the material suspected/known of being contaminated with the fumonisin mycotoxin. In such embodiment, depending on the timing of the addition of the polypeptides of the present disclosure (variants, fragments and/or compositions comprising same) to the material, the detoxification can occur prior to, during and/or after the fermentation.

In an embodiment, the fermentation step is performed to generate the feed ingredient, the feed product, the food ingredient, the food product, the beverage ingredient, and/or the beverage product. In such embodiment, the detoxification step can be performed prior to the fermentation, during the fermentation and/or after the fermentation step. In some specific embodiments, the polypeptides of the present disclosure (variants, fragments and/or compositions comprising same) are contacted with the feed ingredient, the feed product, the food ingredient, the food product, the beverage ingredient, and/or the beverage product prior to and/or during fermentation step to detoxify the fumonisin mycotoxin at least partially during the fermentation step. In some specific embodiments, the beverage ingredient is a beer ingredient, and/or the beverage product is a beer product. In such specific embodiment, the polypeptides of the present disclosure (variants, fragments and/or compositions comprising same) can be contacted with beer ingredient, and/or the beer product to detoxify same. As such, the polypeptides of the present disclosure (variants, fragments and/or compositions comprising same) can be used to make a beer.

Distillers' grains can be obtained during the fermentation of grains (such as corn for example) during the process for making distilled spirits or of biofuels. Brewers' grains during the fermentation of cereal grains (such as malt, barley, wheat, maize, rice, sorghum and/or millet) during the process for making beers. As it is known in the art, distillers' grains as well as brewers' grains have a high nutritional value and can be used as a feed product (alone or combined with other feed product or additives). The method described herein can be applied to distillers' grain (either in a wet or dried form) and/or to brewers' grains (either in a wet or dried forms) to detoxify the fumonisin mycotoxin that may be present. In embodiments in which the polypeptides of the present disclosure (variants, fragments and/or compositions comprising same) are used in the productions of distillers' grains and/or brewers' grains, the polypeptides (variants, fragments and/or compositions comprising same) can be added to biomass intended to be fermented, during fermentation and/or after fermentation.

In an embodiment, distillers' grains and/or brewers' grains can be obtained via a method for making an alcoholic beverage, such as beer or wine or a distilled spirit such as, for example, brandy as well as brandy-based wine, whisky, rum, vodka, gin, tequila, mexcal, sake, or arrack. In such method, a fermenting yeast (which can be the recombinant yeast host cell expressing the polypeptides, variants and/or fragments described herein) contacts the substrate and conducts a fermentation of the substrate. The polypeptides of the present disclosure (variants, fragments and/or compositions comprising same) can be added to the substrate prior to, during and/or after the fermentation. The liquid portion of the fermented substrate can be submitted to a distillation step whereas the solid portion of the fermented substrate can serve as distillers/brewers' grains. When the fermenting agent is a recombinant yeast host cell having expressed and produced the polypeptide, variant or fragment having fumonisin esterase activity, the solid portion of the fermented substrate may have been detoxified during the fermentation and/or can be submitted to a detoxification step directly without the need of adding another source of the heterologous polypeptide having the fumonisin esterase activity. In some embodiments, even when the fermenting agent is a recombinant yeast host cell having expressed and produced the heterologous polypeptide, variant or fragment having fumonisin esterase activity, it may be necessary to add a further source of the heterologous polypeptide having fumonisin esterase activity to the solid portion of the fermented substrate to allow the detoxification of the fermented substrate.

Fermented products, such as alcohols intended to be used biofuels, can be often obtained from fermenting a biomass with a yeast. The fermented product can be an alcohol, such as, for example, ethanol, isopropanol, n-propanol, 1-butanol, methanol, acetone and/or 1, 2 propanediol. In an embodiment, the fermented product is ethanol. The biomass used to produce fermentation products can also be contaminated by fumonisin mycotoxins. Exemplary biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses, or sugar cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and proline-rich proteins). In some embodiments, the substrate comprises starch (in a gelatinized or raw form). In some additional embodiments, the biomass comprises or is derived from corn.

The fermentation step includes contacting a fermenting yeast with an hydrolyzed liquefaction medium or the raw biomass and maintaining this contact under conditions allowing the conversion of the biomass into the fermentation product. In the methods of the present disclosure, the recombinant yeast host cells can be considered as a fermenting yeasts. Alternatively, or in combination, the fermenting yeasts can be used with a composition comprising the polypeptide, variant and/or fragment thereof. Optionally or in combination, the recombinant yeast host cells can be used in a co-culture with fermenting yeasts during the fermentation step. The present disclosure thus provides a fermented biomass comprising the recombinant yeast host cells of the present disclosure or components thereof. The fermenting yeast can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the fermenting yeast is selected from the group consisting of Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the fermenting yeast is of Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the fermenting yeast is Saccharomyces cerevisiae. In some embodiments, the fermenting yeast can be an oleaginous yeast cell. For example, the oleaginous yeast cell can be from the genus Blakeslea, Candida, Cryptococcus, Cunninghamella, Lipomyces, Mortierella, Mucor, Phycomyces, Pythium, Rhodosporidium, Rhodotorula, Trichosporon or Yarrowia. In some alternative embodiment, the fermenting yeast can be an oleaginous microalgae host cell (e.g., for example, from the genus Thraustochytrium or Schizochytrium). In an embodiment, the fermenting yeast is from the genus Saccharomyces and, in some embodiments, from the species Saccharomyces cerevisiae.

The fermentation step can be a batch-fed fermentation, a continuous fermentation or a combination of a plurality of fermentation cycles. In the methods of the present disclosure, the polypeptides, variants and/or fragments as well as combinations thereof can be introduced prior to, during and/or after the fermentation step.

In an embodiment, the polypeptides, variants and/or fragments as well as composition comprising same (such as the recombinant yeast host cells of the present disclosure) are submitted to a plurality of fermentation cycles. The plurality of fermentation cycles in which the recombinant yeast host cells can be submitted comprises at least two distinct fermentation cycles: an initial fermentation cycle and one or more further fermentation cycles. In the initial fermentation cycle, a fermenting population comprising fermenting yeasts (which can be the recombinant yeast host cells or a co-culture of the recombinants yeast host cells and fermenting yeasts) is contacted with a fermentation medium under conditions so as to obtain a fermentation product (and concurrently a fermented medium). The fermenting population obtained at the end of this initial fermentation cycle is recycled for a further fermentation cycle (e.g., substantially isolated and used to inoculate a further fermentation medium). It is recognized that the fermenting population used to inoculate the further fermentation medium can include contaminating wild yeasts which may have been introduced in the fermentation medium of the initial fermentation cycle. The inoculated further fermentation medium is then placed under conditions so as to obtain the fermented product and subsequently substantially isolate a (further) fermenting population (from a further fermented medium). The substantially isolated further fermenting population can be recycled and used to conduct one or more further fermentation cycle. In an embodiment, the polypeptides, variants and/or fragments as well as composition comprising same can be added to the initial cycle, the at least one further cycle or both.

The fermentation step for making the fermented product can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, the fermentation step can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter. Ethanol production can be measured using any method known in the art. For example, the quantity of ethanol in fermentation samples can be assessed using HPLC analysis. Many ethanol assay kits are commercially available that use, for example, alcohol oxidase enzyme-based assays. In some embodiments, the fermenting step is conducted under anaerobic conditions. As described above, yeast tends to undergo fermentation processes while under anaerobic conditions, while it tends to undergo propagation processes while under aerobic conditions. As used herein, “anaerobic conditions” means that the biomass is under an oxygen-poor environment. An oxygen-poor environment may have an oxygen concentration below that of air. For example, the concentration of oxygen may be below 21%, 20%, 15%, 10%, 9%, 8%, 7%, 6%, 5%, 4%, 3%, 2% or 1% by volume.

The methods of the present disclosure can also include a step of separating the fermentation product from other components of the fermented biomass. The methods of the present disclosure can also include a step of obtaining distillers' grains from the fermented biomass.

Optionally, a preliminary liquefaction step can be conducted to hydrolyze, at least in part, the starch molecules which are present, prior to the fermentation step. In the methods of the present disclosure, the polypeptides, variants and/or fragments as well as combinations thereof can be introduced prior to, during and/or after the liquefaction step. A biomass which has been submitted to a liquefaction step can be referred to an hydrolyzed liquefaction medium (which can be an hydrolyzed slurry). The present disclosure thus provides an hydrolyzed liquefaction medium comprising the recombinant yeast host cells described herein or components thereof.

In some embodiments, the polypeptides, variants, fragments and/or compositions comprising same are contacted with a liquefaction medium or a slurry which has not been submitted to a heat treatment step (and in some embodiments which is not intended to be submitted to a heat treatment step). In such instances, the polypeptides, variants, fragments and/or compositions comprising same are contacted with an untreated liquefaction medium or an untreated slurry under a condition so as to generate the hydrolyzed liquefaction medium. In some embodiments, the polypeptides, variants, fragments and/or compositions comprising same are contacted with a liquefaction medium or a slurry which has not yet been submitted to a heat treatment step but is intended to be submitted to such heat treatment step. In such instances, the polypeptides, variants, fragments and/or compositions comprising same are contacted with an untreated liquefaction medium or untreated slurry prior to the heat treatment step. In other embodiments, the polypeptides, variants, fragments and/or compositions comprising same are contacted with a liquefaction medium or a slurry which has already been submitted to a previous heat treatment step. In such instances, the polypeptides, variants, fragments and/or compositions comprising same are contacted with a gelatinized liquefaction medium or gelatinized slurry as the heat treatment would have favored at least partial disruption of the starch molecules which are present in the raw liquefaction medium/raw slurry (to provide a gelatinized liquefaction medium/slurry). In some embodiments, the contact between the gelatinized liquefaction medium and the polypeptides, variants, fragments and/or compositions comprising same can occur during the heat treatment step (at least in part). In some embodiments, the polypeptides, variants, fragments and/or compositions comprising same can be added to the liquefaction medium in the liquefaction process prior to, during and/or after a heat treatment has been applied.

As indicated herein, the liquefaction process can be performed entirely on an untreated liquefaction medium. However, in some embodiments, it may be advantageous to include a heat treatment step to the liquefaction process to liquefy, at least in part, a liquefaction medium comprising gelatinized starch molecules. The heat treatment step can improve the conversion of the starch molecules into dextrins and/or can reduce the time required to complete the liquefaction. The heat treatment step can include submitting the liquefaction medium (which may or may not include the enzyme combination) to a liquefaction temperature and for a liquefaction time period. In some embodiments, the liquefaction of starch occurs in the presence of recombinant microbial host cells and/or the microbial product described herein.

In some embodiments, the liquefaction temperature is at least about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 95° C., 100° C., 105° C. or more can be used. In some further embodiments, the liquefaction temperature is between about 60° C. to 85° C. In some further embodiments, the liquefaction temperature is between about 70° C. to 75° C. In some further embodiments, the liquefaction temperature is between about 80° C. to 85° C. When the liquefaction temperature is between about 60° C. to 85° C. it can be maintained for a liquefaction time of about 60 minutes or more.

In some additional embodiments, a jet cooker can be used to provide the heat treatment step. In such embodiments, the liquefaction temperature can be at least about 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C. or more. Still in such embodiment, the liquefaction temperature can be maintained for a liquefaction time of about 1 minute or more.

### Processes for Making Compositions Comprising Polypeptides, Variants, and Fragments Having Fumonisin Esterase Activity

The present disclosure provides processes for making compositions comprising polypeptides, variants, and fragments having fumonisin esterase activity. In such processes, recombinant microbial host cells comprising a heterologous nucleic acid molecule encoding the polypeptides, variants, and fragments (and thus capable of expressing the polypeptides, variants, and fragments) are propagated to allow the expression and the accumulation of the polypeptides, variants, and fragments. The propagation step is usually conducted in a culture medium allowing the propagation of the recombinant microbial host cell under conditions (agitation, temperature, etc.) to favor the expression of the polypeptides, variants, and fragments having fumonisin oxidase activity. Once the recombinant microbial host cells have been propagated, they can optionally be submitted to an anaerobic growth phase (such as a fermentation step). The processes of the present disclosure also include formulating the propagated and optionally fermented recombinant microbial host cells into a composition comprising the polypeptides, variants, and fragments.

In some embodiments, the processes include a step of separating one or more components of the propagated recombinant microbial host cell from the polypeptides, variants, and fragments to obtain a separated fraction enriched in polypeptides, variants, and fragments. When the polypeptides, variants, and fragments are expressed in a secreted (free) form, the dissociation step can include, for example, a filtration or a centrifugation step to obtain the separated fraction.

When the polypeptides, variants, and fragments are expressed intracellularly or associated with the membrane (and in some embodiments tethered), the polypeptides, variants, and fragments do not necessarily need to be separated from the recombinant microbial host cells (or components thereof). Instead, the propagated recombinant microbial host cells can be submitted to a lysis step to provide a lysed fraction. The lysis step can be achieved, for example, by autolysis, a heat treatment, a pH treatment, a salt treatment, a homogenization step, etc. Alternatively, the propagated recombinant microbial host cells can be submitted to an inactivation step to provide an inactivated or semi-inactive fraction. The inactivation step can be achieved, for example, by autolysis, a heat treatment, a pH treatment, a salt treatment, a homogenization step, etc.

In some embodiments, the propagated recombinant microbial host cell, the separated fraction, the lysed fraction and/or the inactivated or semi-inactive fraction can be submitted to a drying step to obtain a dried fraction. In additional embodiments, the process can include one or more washing steps to obtain the composition. The process can further include, in some embodiments, a heating step to provide the composition. The process can include, in some embodiment, determining the purity and/or the activity of the polypeptides, variants, and fragments having fumonisin esterase activity which are present in the composition.

The processes of the present disclosure can also include a step of introducing one or more copies of a heterologous nucleic acid in a microbial cell to obtain a recombinant microbial host cell capable of expressing the polypeptide, the variant, and the fragment of the present disclosure. The recombinant microbial host can be a yeast host cell, a fungal host cell or a bacterial host cell. In an embodiment, the recombinant yeast host cell is from the genus Saccharomyces and, in some additional embodiments, from the species Saccharomyces cerevisiae. The recombinant yeast host cell can be from the genus Pichia and, in some additional embodiments, from the species Pichia pastoris. The recombinant fungal host cell can be from the genus Aspergillus or Trichoderma. The recombinant bacterial host cell can be from the genus Bacillus, and in some additional embodiments, from the species Bacillus subtilis. The recombinant bacterial host cell can be from the genus Escherichia, and in some additional embodiments, from the species Escherichia coli. The recombinant bacterial host cell can be from the genus Lacticaseibacillus, and in some additional embodiments, from the species Lacticaseibacillus casei. The present disclosure can include of step of determining the presence of the heterologous nucleic acid molecule in the recombinant microbial host cell and/or the ability of the recombinant microbial host cell to express the polypeptide, variant or fragment of the present disclosure.

The composition can be provided in a liquid, semi-liquid or dry form. The composition can be a bacterial composition (e.g., a composition made from a recombinant bacterial host cell having expressed the polypeptide, variant, or fragment of the present disclosure). The composition can be a yeast composition (e.g., a composition made from a recombinant yeast host cell having expressed the polypeptide, variant, or fragment of the present disclosure). The composition can be a fungal composition (e.g., a composition made from a recombinant fungal host cell having expressed the polypeptide, variant, or fragment of the present disclosure).

The process can also be used to make a yeast product (e.g., a composition derived from a recombinant yeast host cell having expressed the polypeptide, variant, or fragment of the present disclosure). When the yeast product is an inactivated yeast product, the process for making the yeast product broadly comprises two steps: a first step of providing propagated recombinant yeast host cells and a second step of lysing the propagated yeast host cells for making the yeast product. The process for making the yeast product can include an optional separating step and an optional drying step. In some embodiments, the propagated recombinant yeast host cells are propagated on molasses. Alternatively, the propagated recombinant yeast host cells are propagated on a medium comprising a yeast extract.

In some embodiments, the recombinant yeast host cells can be lysed using autolysis (which can optionally be performed in the presence of additional exogenous enzymes). For example, the propagated recombinant yeast host cells may be subject to a combined heat and pH treatment for a specific amount of time (e.g., 24 h) in order to cause the autolysis of the propagated recombinant yeast host cells to provide the lysed recombinant yeast host cells. For example, the propagated recombinant yeast host cells can be submitted to a temperature of between about 40° C. to about 70° C. or between about 50° C. to about 60° C. The propagated recombinant yeast host cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant yeast host cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant yeast host cells can be submitted to a pH between about 4.0 and 8.5 or between about 5.0 and 7.5. The propagated recombinant yeast host cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant yeast host cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6 or 4.5.

In some embodiments, the recombinant yeast host cells can be homogenized (for example using a bead-milling technique, a bead-beating or a high-pressure homogenization technique) and as such the process for making the yeast product comprises a homogenizing step.

The process for making the yeast product can also include a drying step. The drying step can include, for example, roller-drying, electrospray-drying, freeze-drying, spray-drying, lyophilization and/or fluid-bed drying. When the yeast product is an autolysate, the process may include directly drying the lysed recombinant yeast host cells after the lysis step without performing an additional separation of the lysed mixture.

The process for making the yeast product can include a heating step. In such embodiment, care should be taken to perform the heating step so as to preserve the residual enzymatic activity of the polypeptides of the present disclosure.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product. When the yeast composition is an active/semi-active product, it can be submitting to a concentrating step, e.g., a step of removing part of the propagation/fermentation medium from the propagated recombinant yeast host cells. The concentrating step can include resuspending the concentrated and propagated/fermented recombinant yeast host cells in the propagation medium (e.g., unwashed preparation) or a fresh medium or water (e.g., washed preparation). The process can also be used to make a bacterial product (e.g., a composition derived from a recombinant bacterial host cell having expressed the polypeptide, variant, or fragment of the present disclosure). When the bacterial product is an inactivated bacterial product, the process for making the bacterial product broadly comprises two steps: a first step of providing propagated recombinant bacterial host cells and a second step of lysing the propagated bacterial host cells for making the yeast product. The process for making the bacterial product can include an optional separating step and an optional drying step. In some embodiments, the recombinant bacterial host cells are propagated on a medium comprising a yeast extract. In some embodiments, the recombinant bacterial host cells can be homogenized (for example using a bead-milling technique, a bead-beating or a high-pressure homogenization technique) and as such the process for making the bacterial product comprises a homogenizing step.

The process for making the bacterial product can also include a drying step. The drying step can include, for example, roller-drying, electrospray-drying, freeze-drying, spray-drying, lyophilization and/or fluid-bed drying. When the bacterial product is an autolysate, the process may include directly drying the lysed recombinant bacterial host cells after the lysis step without performing an additional separation of the lysed mixture.

The process for making the bacterial product can include a heating step. In such embodiment, care should be taken to perform the heating step so as to preserve the residual enzymatic activity of the polypeptides of the present disclosure.

When the bacterial composition is an active/semi-active product, it can be submitting to a concentrating step, e.g., a step of removing part of the propagation/fermentation medium from the propagated recombinant bacterial host cells. The concentrating step can include resuspending the concentrated and propagated recombinant bacterial host cells in the propagation/fermentation medium (e.g., unwashed preparation) or a fresh medium or water (e.g., washed preparation).

The process can also be used to make a fungal product (e.g., a composition derived from a recombinant fungal host cell having expressed the polypeptide, variant, or fragment of the present disclosure). When the fungal product is an inactivated fungal product, the process for making the fungal product broadly comprises two steps: a first step of providing propagated recombinant fungal host cells and a second step of lysing the propagated fungal host cells for making the fungal product. The process for making the fungal product can include an optional separating step and an optional drying step. In some embodiments, the propagated recombinant fungal host cells are propagated on molasses. Alternatively, the recombinant fungal host cells are propagated on a medium comprising a fungal extract.

In some embodiments, the recombinant fungal host cells can be homogenized (for example using a bead-milling technique, a bead-beating or a high-pressure homogenization technique) and as such the process for making the fungal product comprises a homogenizing step.

The process for making the fungal product can also include a drying step. The drying step can include, for example, roller-drying, electrospray-drying, freeze-drying, spray-drying, lyophilization and/or fluid-bed drying. When the fungal product is an autolysate, the process may include directly drying the lysed recombinant fungal host cells after the lysis step without performing an additional separation of the lysed mixture.

The process for making the fungal product can include a heating step. In such embodiment, care should be taken to perform the heating step so as to preserve the residual enzymatic activity of the polypeptides of the present disclosure.

When the fungal composition is an active/semi-active product, it can be submitting to a concentrating step, e.g., a step of removing part of the propagation/fermentation medium from the propagated/fermented recombinant fungal host cells. The concentrating step can include resuspending the concentrated and propagated recombinant fungal host cells in the propagation/fermentation medium (e.g., unwashed preparation) or a fresh medium or water (e.g., washed preparation).

In an aspect, the process can include admixing the polypeptides, variants, and fragments of the present disclosure with a further component such as, for example, a binder (mineral and/or organic), a further polypeptide having the ability to detoxify a mycotoxin, a microbe (bacterium, yeast, fungus), a microbial component, a food product, a feed product and/or a beverage product. The compositions of the present disclosure may be combined with a culture medium (used or intended to be used with the microbial host cell), a liquefaction medium and/or a fermentation medium.

In an embodiment, the process can include admixing the compositions of the present disclosure with a further component. In an embodiment, this further component is a binder. As used in the present disclosure, a “binder” is a component capable of physically associated (e.g., binding) to a mycotoxin to reduce its bioavailability. The binder can include a mineral binder, an organic binder or both. An exemplary mineral binder is a clay, such as a bentonite clay. An exemplary organic binder is a microbial component, such as, for example, a microbial extract (e.g., a bacterial extract, a yeast extract and/or a fungal extract).

In another embodiment, this further component is a microbial cell and/or a component from a microbial cell. The microbial cell can be a bacterial cell, a yeast cell or a fungal cell. The microbial cell can be a recombinant microbial host cell capable of expressing the polypeptide, the variant, or the fragment of the present disclosure. The microbial host cell can be a recombinant microbial host cell having expressed and optionally accumulated the polypeptide, the variant, or the fragment of the present disclosure.

In still another embodiment, this further component is a polypeptide capable of detoxifying a mycotoxin, such as the fumonisin mycotoxin. The polypeptide capable of detoxifying a mycotoxin may be a polypeptide having fumonisin esterase activity, which may be, in still another embodiment, one or more of the polypeptides, variants, or fragments described herein. Additional embodiments having fumonisin esterase activity include, but are not limited to, those described in WO199606175 as well as in U.S. Pat. No. 6,025,188, both of which are incorporated herewith in their entirety. The polypeptide capable of detoxifying a mycotoxin may be, in some embodiments, a polypeptide having fumonisin amine oxidase activity. Embodiments of polypeptides having fumonisin amine oxidase activity include, but are not limited to, the polypeptides described in WO2020047656, which is herewith incorporated in its entirety.

The process of the present disclosure can be used for making a food product. In such embodiment, the composition of the present disclosure can be combined with a food ingredient either to detoxify it or to eventually be combined with other food ingredients to make the food product. The composition of the present disclosure can also be used as a food additive. The process of the present disclosure can include formulating the compositions in a powder form, such as a free-flowing powder, and using this powder form in the preparation of the food product. The process of the present disclosure can include formulating the compositions in a liquid form, which can eventually be dried on or in the food product. The process for making the food product can include a fermenting step, a freezing step, a heat treatment step, etc.

The process of the present disclosure can be used for making a feed product. In such embodiment, the composition of the present disclosure can be combined with a feed ingredient either to detoxify it or to eventually be combined with other feed ingredients to make the feed product. The composition of the present disclosure can also be used as a feed additive. For example, in some embodiments, the compositions can be combined with the feed product prior to consumption by the animals. The process of the present disclosure can include formulating the compositions in a powder form, such as a free-flowing powder, and using the powder form in the preparation of the feed product. The process of the present disclosure can include formulating the compositions in a liquid form, which can eventually be dried on and/or in the feed product. For example, in some embodiments, the process can include forming feed pellets and coating such pellets with a composition in a liquid or semi-liquid form. The process for making the feed product can include a fermenting step, a freezing step, a heat treatment step, etc. In some embodiments, the process for making a feed pellet including applying a heat treatment to the feed pellet (which can include the polypeptide, variant, fragment and/or composition described herein. In an embodiment, the heat treatment used in the process is conducted at a temperature between 5° and 95° C., and in some additional embodiments, at a temperature between 5° and 85° C. In a specific embodiment, the process is conducted at a temperature of at least 50, 55, 60, 65, 70, 75, 80, 85, 90, 95° C. or more. In yet another embodiment, the process is conducted at a temperature of at least 50° C. In another specific embodiment, the process is conducted at a temperature of no more than 95, 90, 85, 80, 75, 70, 65, 60, 55, 50° C. In still yet a further embodiment, the process is conducted at a temperature of no more than 85° C. In still yet a further embodiment, the process is conducted at a temperature of no more than 80° C. In yet another specific embodiment, the process is conducted at a temperature between about 50, 55, 60, 65, 70, 75, 80, 85, or 90° C. and 95, 90, 85, 80, 75, 70, 65, 60, 55° C.

The process of the present disclosure can be used for making a beverage product. In such embodiment, the composition of the present disclosure can be combined with a beverage ingredient either to detoxify it or to eventually be combined with other beverage ingredients to make the beverage product. The composition of the present disclosure can also be used as a beverage additive. The process of the present disclosure can include formulating the compositions in a powder form, such as a free-flowing powder, and using the powder form in the preparation of the beverage product. The process of the present disclosure can include formulating the compositions in a liquid form to include it in the beverage product. The process for making the beverage product can include a fermenting step, a freezing step, a heat treatment step, a distillation step, etc.

### EXAMPLES

### Example 1: Modification, Cloning and Expression of Polynucleotides Encoding Polypeptides Exhibiting Fumonisin Esterase (FE) Activity

The polypeptides exhibiting FE activity including truncations at the N- and/or C-termini were obtained using nested Polymerase Chain Reaction (PCR). Libraries of mutant polypeptides exhibiting FE activity libraries was obtained by introducing random mutations within the nucleotide sequences by error-prone PCR as well as combinatorial methods.

### Example 2: Determination of the Catalytic Activity and Specific Activity of Polypeptides Exhibiting Fumonisin Esterase (FE) Activity

MUA assay: 4-methylumbelliferyl acetate (MUA assay) was developed to increase FE variants screening throughput. MUA contains 4-methylumbelliferone moiety and 7-O-acetyl group. When the acetyl group is cleaved off, the fluorescent 4-methylumbelliferone moiety is measured using a spectrophotometer (ex/em=360/450 nm). The MUA assay detects purified FE variants linearly from 14 to 1000 ng/ul of in a YPD40 supernatant. Fluorescence reading from MUA assay also highly correlates (R2=0.89) with LCMS HFB1 measurement provided by the TCA assays. Briefly, FE-producing host cells in YPD40 culture medium (500 μL) were inoculated in a 96 deep-well plate and were allowed to grow overnight (30° C., 900 rpm). MUA was dissolved in DMSO (200 mM) and then diluted in 25 mM Tris pH7.5 or in HEPES 50 mM pH7-7.5 to reach a final concentration of 200 uM. MUA solution (95 uL) and culture media supernatants (5 uL) were mixed, incubated at 37° C. for 15 min with agitation (250 rpm), and then fluorescence was measured (360 nm excitation and 450 nm emission) with a plate reader. Best candidates (higher fluorescence activity) were retained for further tricarballylic acids (TCA) assays. For experiments including a heat treatment: supernatants (50 uL) were transferred into a 96 well PCR plate for 5 min incubation at the desire temperature (i.e., heat shock for thermostability assays) before taking 5 ul for the MUA assay.

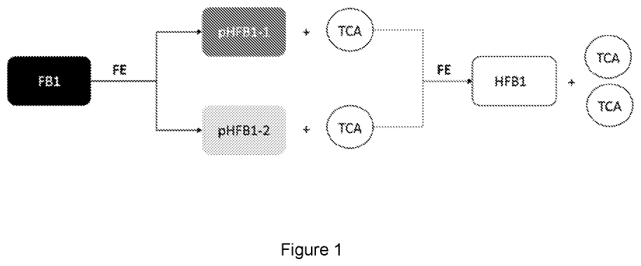

TCA assay: Fumonisin (FB1) is hydrolyzed by the polypeptides having fumonisin esterase activity (FE) disclosed in the invention herein into two tricarballylic acids (TCA) and hydrolyzed fumonisin (HFB1), as depicted in FIG. 1. Since TCA is released during the fumonisin hydrolysis (FIG. 1), the TCA concentration is a direct indicator of FE activity. The enzyme unit (U) is defined as umol of TCA released per min (umol/min) in 20 mM Tris pH 8 with 100 uM FB1. To conduct the FE activity assay, overnight YPD40 supernatant was diluted between 100 and 1000-fold and 10 μL of the diluted supernatant was added into 90 ul of 20 mM Tris-HCl pH 8 with 0.1 mg/mL BSA and 75 ppm (100 uM) of FUM B1 (final concentration). The sample was incubated at 30° C. (unless otherwise indicated) for exactly 10 minutes followed by 99° C. for 15 minutes (to stop the enzyme activity) and then submitted for Q Exactive Orbitrap LC-MS analysis to determine the amount of FB1 and its degradation products (pHFB1-1, pHFB1-2, HFB1, and TCA).

### Example 3: Heterologous Expression of Fumonisin Esterase (FE) in a Recombinant Yeast Host Cell

The polypeptide having a FE activity having the amino acid sequence of SEQ ID NO: 3 was expressed, in various forms in a S. cerevisiae host cell. Table 1 provides the genotype of the various Saccharomyces cerevisiae strains tested in this Example.

Table 1. Genotype of the strains/transformants used in Example 3. All strains/transformants, except X33.1, were all derived from the S. cerevisiae parental (and non-genetically modified) strain M10580. Strains X33.1 was obtained from genetically modifying Komagataella phaffii strain X33 (obtained from Invitrogen).

| Designation | Heterologous polypeptide(s) expressed |
|---|---|
| M10580 | None - this is the parental yeast |
| M22810 | Intracellular form of the fumonisin esterase of SEQ |
| | ID NO: 3 on expression cassette |
| | Genome integration of expression cassette |
| M22939 | Tethered form of the fumonisin esterase of SEQ ID |
| | NO: 3 (using an AGA1/AGA2 tethering moiety |
| | having the amino aid sequence of SEQ ID NO: 34) |
| | on expression cassette |
| | Genome integration of expression cassette |
| M20435 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Genome integration of expression cassette |
| T8612-1 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Expression of expression cassette from a plasmid |
| T8612-2 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Expression of expression cassette from a plasmid |
| T8612-3 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Expression of expression cassette from a plasmid |
| T8612-4 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Expression of expression cassette from a plasmid |
| X-33.1 | Secreted form of the fumonisin esterase of SEQ ID |
| | NO: 3 (using an alpha mating factor 1 signal |
| | sequence having the amino acid sequence of SEQ |
| | ID NO: 39) on expression cassette |
| | Expression of expression cassette from a plasmid |

Strains M10580, M22810, M22939 and M20435 were cultured in YPD40 culture medium for 48 hours to reach saturation (DCW ˜10.5 g/L). 400 μL of culture of strain M22810 was lysed with 20-second bead beating, and 10 μL of the debris-free supernatant was used in the assay, while 10 μL of M22939 cell suspension or 10 ul of culture supernatant of M20435 were used. The sample was added into 90 μL of 25 mM Tris buffer at pH 7.5 with 100 ppm of FB1. The resulting mixture was then incubated at 37° C. for 15 minutes followed by heat inactivation. LCMS analysis of FB1 and its derivatives (pHFB1-1, pHFB1-2 and HFB1) was performed.

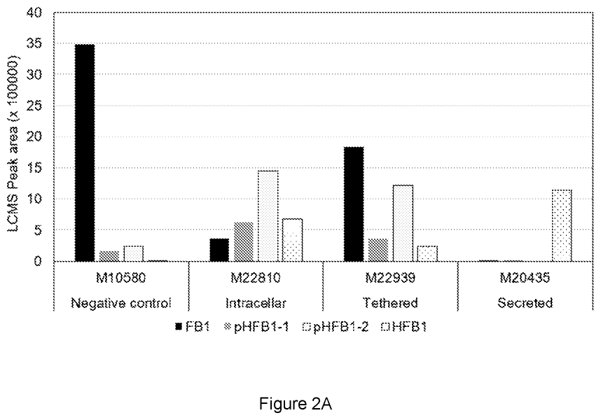

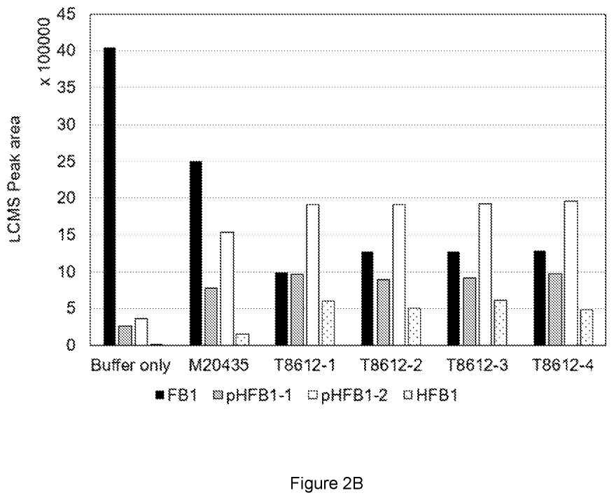

It was estimated that strain M20435 was able to achieve FE expression level at 3.33 mg/g DCW or 6.7 mg/L of supernatant (data not shown). It was also determined that strains M22810 (intracellular form) and M22939 (tethered form) also exhibited fumonisin esterase activity (as determined by LCMS, as shown on FIG. 2A). As shown in FIG. 2A, the FE expressed by strain M20435 completely hydrolyzed FB1 into HFB1 while the FE expressed by strains M22810 and M22939 hydrolyzed 90% and 47% of FB1, respectively. There were also high-level of pHFB1 present in the samples obtained from the FE expressed by strains M22810 and M22939.

Plasmid pMU4761 was constructed with an identical expression cassette as strain M20435. Four clones from a transformation (T8612-1, -2, -3 and -4) were examined using Western blotting and LCMS analysis. Based on the western blot densitometry analysis, four transformants averaged 1.45±0.25 times higher FE3 expression than M20435 (data not shown). LCMS analysis also showed that T8612 strains reduced an additional 32±3% of FB1 compared to M20435 (see FIG. 2B).

FE was cloned into pPICZ-α plasmid containing a-mating factor secretion peptide expression cassette from an Invitrogen Pichia expression kit. The resulting plasmid, pMU4736, was integrated into Komagataella phaffiix-33 to generate strain X-33.1. Methanol-inducible promoter, AOX1, was used to drive FE expression. The FE expression and function were verified via western blotting and LCMS analysis. The expression level of the resulting FE-expressing Pichia strain was estimated at 4 mg/g DCW or 20 mg/L supernatant.

### Example 4: Characterization of Truncated Forms Derived from a Polypeptide Fumonisin Esterase (FE) Activity

Preliminary characterizations of the polypeptide of SEQ ID NO: 35 were undertaken, and it was determined that this polypeptide loses >50% of its activity after 1-hour heat shock at 50° C. or two minutes at 70° C. (data not shown). To improve the enzymatic activity (especially after a heat challenge) of polypeptide of SEQ ID NO: 35, the effect of performing N- and/or C-terminal truncations was investigated. As indicated in Example 1, a series of nested truncated were made to generate such truncated forms of SEQ ID NO: 35. The truncated forms were expressed in S. cerevisiae and the FE activity, at 37° C., of the resulting supernatant was determined. Table 2 summarizes the results that have been obtained.

Table 2. Description of the various deletions made and effect on FE enzymatic activity as measured at 37° C. The truncations were made from a polypeptide having the amino acid sequence of SEQ ID NO: 35.

| | # of amino acid | # of amino acid | |
|---|---|---|---|
| | residues | residues | Effect on activity (when |
| | truncated from | truncated from | compared to activity of |
| Designation | N-terminus | C-terminus | SEQ ID NO: 35) |
| N10 | 10 | None | Similar |
| N12 | 12 | None | Similar |
| N14 | 14 | None | Similar |
| N16 | 15 | None | Reduced |
| N18 | 18 | None | No detectable activity |
| N20 | 20 | None | No detectable activity |
| N22 | 22 | None | No detectable activity |
| N30 | 30 | None | No detectable activity |
| N40 | 40 | None | No detectable activity |
| N50 | 50 | None | No detectable activity |
| C50 | None | 50 | No detectable activity |
| C40 | None | 40 | No detectable activity |
| C30 | None | 30 | No detectable activity |
| C22 | None | 22 | No detectable activity |
| C20 | None | 20 | No detectable activity |
| C18 | None | 18 | Reduced |
| C16 | None | 16 | Reduced |
| C14 | None | 14 | Reduced |
| C12 | None | 12 | Reduced |
| C10 | None | 10 | Reduced |
| C8 | None | 8 | Reduced |
| C6 | None | 6 | Reduced |
| C4 | None | 4 | Similar |
| N10/C4 | 10 | 4 | Similar |
| N12/C4 | 12 | 4 | Similar |

As indicated in Table 2, the 12 amino acid N-terminal and 4 amino acid C-terminal truncation (noted N12/C4) is the smallest variant with the same catalytic activity as the full-length FE having SEQ ID NO: 35 at 37° C. Surprisingly, no thermostability improvement was observed in the N12/C4 variant (data not shown).

### Example 5: Polypeptides Having Increased Fumonisin Esterase Activity

A first library of random mutant polypeptides was generated, as indicated in Example 1, using the control polypeptide of SEQ ID NO: 3 (which includes a 12-amino acid truncation at its N-terminus, when compared to SEQ ID NO: 35). Some of the mutant polypeptides of this first library were expressed in S. cerevisiae in a secreted form, and their respective supernatants were screened for their ability to exhibit fumonisin esterase activity at 37° C. Table 3 summarizes a selection of these mutants characterized in the present example.

Table 3. Description of the control and various mutant polypeptides obtained in the 1^{st} error-prone PCR screen.

| | Designation of mutant | SEQ ID NO of mutant |
|---|---|---|
| | polypeptide having | polypeptide having |
| | fumonisin esterase | fumonisin esterase |
| Strain | activity | activity |
| M20435 | 102101F5 (control) | SEQ ID NO: 3 |
| M23835 | 112502A10 | SEQ ID NO: 8 |
| M23837 | 113002H1 | SEQ ID NO: 36 |
| M23401 | 103002H2 | SEQ ID NO: 7 |
| M23283 | T25 or N10/C4 | SEQ ID NO: 37 |
| M23399 | 102701E2 | SEQ ID NO: 6 |
| M23849 | 120301C6 | SEQ ID NO: 9 |
| M24312 | 120301C6 or R307P | SEQ ID NO: 38 |

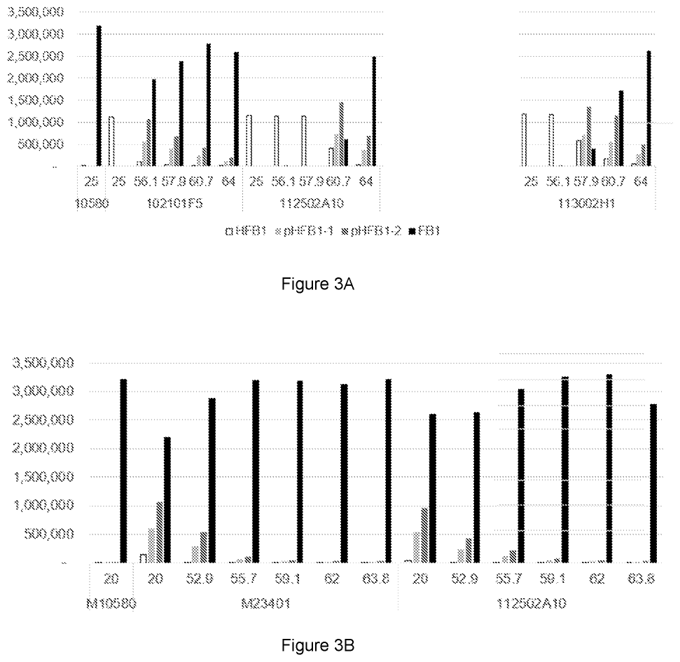

The supernatant of selected strains having expressed mutant polypeptides which did exhibit fumonisin esterase activity at 37° C. was further characterized after having been submitted to a heat challenge. More specifically, the supernatant of the culture of the selected strains was heated at a temperature of 56.1, 57.9, 60.7 or 64° C. for 5 minutes. Then, 4 μL of the heat-treated supernatant was added to 96 μL of 25 mM Tris HCl (pH 7.5) supplemented with fumonisin B1 (100 ppm). The resulting mixture was incubated for 15 minutes at 37° C., followed by a further incubation at 99° C. for a further 15 minutes. The amount of each of the fumonisin metabolites was determined using LCMS. As shown in FIGS. 3A and 3B, the selected mutants (112502A10, 113002H1, 103002H2=M23401) exhibited increased FE activity following a heat challenge compared to the control (102101F5).

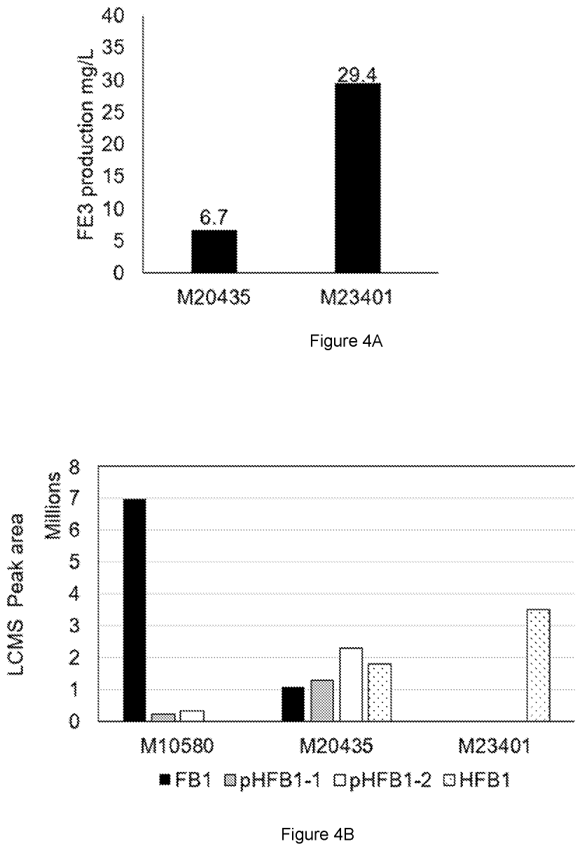

The supernatant of the strains selected having expressed mutant polypeptides which did exhibit fumonisin esterase activity at 37° C. was further characterized to determine the amount (as determined by Western blot) and activity (as determined by LCMS) of the mutant polypeptides. As shown on FIG. 4A, the supernatant of the culture of strains M23401 had an increased amount of the polypeptide having fumonisin esterase activity when compared to the supernatant of the culture of strain M20435. As shown on FIG. 4B, the supernatant of the culture of strains M23401 had an increased fumonisin esterase activity when compared to the supernatant of the culture of strain M20435.

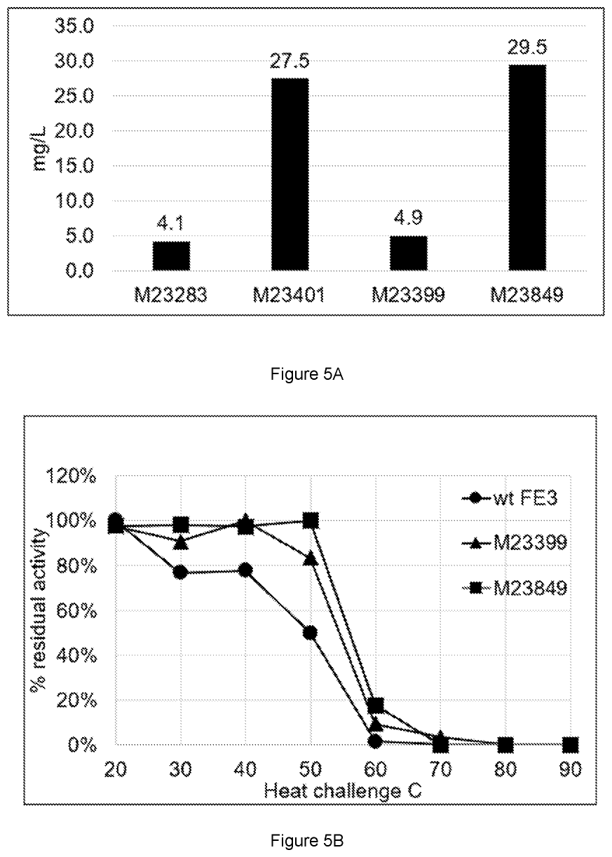

The supernatant of additional strains selected having expressed mutant polypeptides which did exhibit fumonisin esterase activity at 37° C. was further characterized to determine the amount (as determined by Western blot) and activity (as determined by LCMS) of the mutant polypeptides. As shown on FIG. 5A, the supernatant of the culture of strains M23401, M23399 and M23849 had an increased amount of the polypeptide having fumonisin esterase activity when compared to the supernatant of the culture of strain M23283. The mutant polypeptides were submitted to a heat challenge as described above and their activity was determined. As shown on FIG. 5B, the mutant polypeptides present in the supernatants of strains M23399 and M23849 exhibited higher temperature tolerance during the heat challenge when compared to those present in the supernatant of strain M20435 (referred to as wt FE3 on FIG. 5B).

A second library of mutant polypeptides was generated, as indicated in Example 1, using the best polypeptide identified in the first library, namely 120301C6. Some of the mutant polypeptides of this second library were expressed in S. cerevisiae in a secreted form, and their respective supernatants were screened for their ability to exhibit fumonisin esterase activity at 37° C. The top 22 clones, based on their fumonisin esterase activity at 37° C., obtained from this second round of error-prone PCR are presented in Table 4.

Table 4. Description of the mutant polypeptides obtained in the 2^{nd} error-prone PCR screen. The second library was generated using polypeptide 120301C6 as the parent.

| | Designation of mutant | SEQ ID NO of mutant |
|---|---|---|
| | polypeptide having | polypeptide having |
| | fumonisin esterase | fumonisin esterase |
| Strain | activity | activity |
| — | T3A3 | SEQ ID NO: 15 |
| — | S05D6 | SEQ ID NO: 16 |
| — | T5C9 | SEQ ID NO: 17 |
| — | T1B7 | SEQ ID NO: 18 |
| — | T1A6 | SEQ ID NO: 19 |
| — | T1A5 | SEQ ID NO: 21 |
| — | T3G7 | SEQ ID NO: 20 |
| — | T5B9 | SEQ ID NO: 22 |
| — | T1B9 | SEQ ID NO: 23 |
| — | T5G5 | SEQ ID NO: 24 |
| M24869 | S03D2 | SEQ ID NO: 11 |
| — | S01G6 | SEQ ID NO: 26 |
| — | S05B1 | SEQ ID NO: 25 |
| — | T7A3 | SEQ ID NO: 27 |
| — | T4C11 | SEQ ID NO: 28 |
| — | S01C12 | SEQ ID NO: 29 |
| — | T7D7 | SEQ ID NO: 30 |
| M24870 | T4G8 | SEQ ID NO: 12 |
| — | S03C10 | SEQ ID NO: 31 |
| M24868 | S01D3 | SEQ ID NO: 14 |
| — | S01G8 | SEQ ID NO: 32 |
| M24871 | T7F4 | SEQ ID NO: 13 |

The supernatant of S. cerevisiae strains having expressed a secreted form of the mutant polypeptides of Table 4 were submitted to a heat challenge (as described above) at 45, 50, 55, 60, 65 or 70° C. for 5 minutes. The fumonisin esterase activity was determined in the heat-challenged supernatant and compared with the supernatant of a S. cerevisiae strain expressing 120301C6 (referred to as the parent in Table 4). The results of these heat challenges are presented in Table 5.

The occurrence of amino acid substitutions (when compared to 120301C6) of various mutant polypeptides described in Table 4 was determined and is presented in Table 6.

Table 5. Percent of residual active of the mutant polypeptides presented in Table 4 after a 5 min heat shock as determined by MUA assay. Average of four biological replicates normalized to the parent polypeptide (M24312 expressing 120301C6, labelled as P in the table below) having been submitted to a heat challenge. N=negative control.

TABLE 5
Percent of residual active of the mutant polypeptides presented in Table 4 after a 5 min heat shock as
determined by MUA assay. Average of four biological replicates normalized to the parent polypeptide (M24312
expressing 120301C6, labelled as P in the table below) having been submitted to a heat challenge.

| Heat Shock | Average (in %) | | | | | | | | | | | |
|---|---|---|---|---|---|---|---|---|---|---|---|---|
| (° C.) | N | P | T3A3 | S05D6 | T5C9 | T1B7 | T1A6 | T1E5 | T3G7 | T5B9 | T1B9 | T5G5 |
| 45 | 8 | 100 | 89 | 76 | 103 | 97 | 110 | 105 | 116 | 122 | 112 | 124 |
| 50 | 9 | 108 | 95 | 77 | 110 | 98 | 109 | 110 | 134 | 128 | 115 | 128 |
| 55 | 9 | 98 | 91 | 78 | 106 | 101 | 115 | 108 | 123 | 125 | 114 | 128 |
| 60 | 8 | 56 | 72 | 66 | 89 | 83 | 94 | 88 | 101 | 106 | 95 | 109 |
| 65 | 8 | 17 | 39 | 43 | 43 | 46 | 55 | 57 | 59 | 60 | 62 | 64 |
| 70 | 8 | 9 | 10 | 12 | 11 | 10 | 11 | 11 | 14 | 12 | 11 | 12 |

| Heat Shock | Average (in %) | | | | | | | | | | | | | |
|---|---|---|---|---|---|---|---|---|---|---|---|---|---|---|
| (° C.) | N | P | SC3D2 | S01G6 | S05B1 | T7A3 | T4C11 | S01C12 | T7D7 | T4G8 | S03C10 | S01D3 | S01G8 | T7F4 |
| 45 | 8 | 100 | 119 | 116 | 129 | 117 | 113 | 127 | 120 | 123 | 130 | 129 | 134 | 125 |
| 50 | 9 | 108 | 126 | 124 | 135 | 118 | 120 | 135 | 117 | 131 | 138 | 138 | 143 | 121 |
| 55 | 9 | 98 | 126 | 123 | 134 | 121 | 115 | 133 | 120 | 128 | 136 | 135 | 143 | 128 |
| 60 | 8 | 56 | 111 | 110 | 118 | 105 | 104 | 114 | 106 | 112 | 121 | 121 | 127 | 114 |
| 65 | 8 | 17 | 67 | 69 | 73 | 74 | 76 | 76 | 77 | 78 | 78 | 82 | 83 | 89 |
| 70 | 8 | 9 | 14 | 13 | 12 | 15 | 19 | 11 | 16 | 20 | 13 | 14 | 14 | 19 |

N = negative control.

Table 6. Summary of mutations found in the mutant polypeptides of Table 4 clones arranged in order of increased residual activity after a 5 min 65° C. heat challenge (left to right). #=number times sequence is found in the mutant polypeptides characterized.

TABLE 6
Summary of mutations found in the mutant polypeptides of Table 4 clones arranged in order
of increased residual activity after a 5 min 65° C. heat challenge (left to right).

| | | | | | | | | | | | | | | | Occurrence |
|---|---|---|---|---|---|---|---|---|---|---|---|---|---|---|---|
| | T3A3 | S05D6 | T5C9 | T1B7 | T1A6 | T1E5 | T3G7 | T5B9 | T1B9 | T5G5 | T4G8 | S03C10 | S01G8 | T7F4 | Rate |
| # | 1 | 1 | 1 | 1 | 1 | 1 | 1 | 1 | 1 | 1 | 1 | 2 | 5 | 4 | |
| V11 | | | | | | | | | | | | | | | |
| M19 | | I | | | I | I | I | I | I | | | I | I | I | 77% |
| R22 | K | | | K | K | K | | K | K | K | K | | | | 36% |
| G33 | | D | | | D | | D | D | | D | | D | D | D | 73% |
| H41 | | | | | | | | | | | | | | | 0% |
| G87 | D | | D | D | | | D | D | | D | D | D | D | D | 82% |
| F129 | | | | | | | | | | | | | | | 0% |
| L136 | F | F | F | F | F | F | F | F | F | F | F | F | F | F | 100% |
| R173 | I | I | | I | | | I | I | | I | | | | I | 45% |
| A284 | X | | | | D | D | D | D | X | D | | D | D | D | 82% |
| A287 | | | | | | | | V | | V | | | | | 9% |
| R295 | P | P | P | P | P | P | P | P | P | P | P | P | P | P | 100% |
| P313 | | S | | | | | S | | | | | | | S | 27% |
| M314 | L | I | L | L | L | L | | I | L | I | I | I | I | I | 95% |
| G389 | D | D | D | D | D | D | D | D | D | D | D | D | D | D | 100% |
| P424 | | | L | | | | | L | | L | | L | L | | 45% |
| E468 | K | K | | K | | | K | | | | | | | K | 36% |
| P482 | S | | | S | S | S | | | S | | | | S | | 45% |

# = number times sequence is found in the mutant polypeptides characterized.

### Example 6: Treatment of Corn Silage with a Polypeptide Having Fumonisin Esterase Activity

High moisture corn (HMC, 1.35 kg in total) stored at −20° C. was artificially contaminated with fumonisin (SML1286, Sigma Aldrich) at the rate of 2 mg/kg with a sprayer in a big bag. Three different dosages of an isolated polypeptide having a fumonisin esterase activity (having the amino acid sequence of SEQ ID NO: 14 at 3200 U/g) were tested: 0 U/kg (Samples A1-3), 40 U/kg (Samples B1-3) and 80 U/kg (Samples C1-3). For each treatment, one time point was prepared (115 days) and 3 replicates were prepared. For each treatment, 0.242 kg of artificially contaminated HMC was used and split to 3 replicates (0.075 kg/replicate).

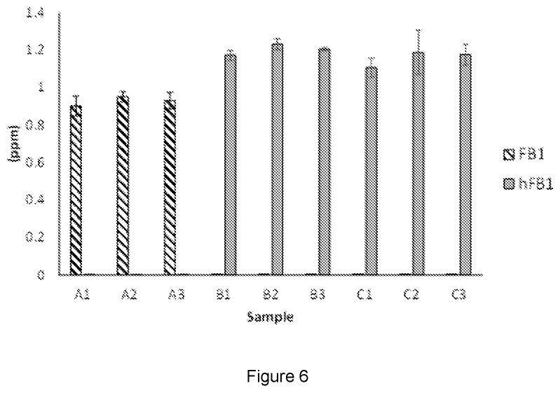

Each sample was mixed well and analyzed in triplicate sub-samples by LC-MS/MS using matrix matched calibration curves. The overall concentrations detected in subsamples supported good sample homogeneity. FB1 concentrations were reduced in samples B and C, coinciding with the appearance of HFB1. There was no significant difference observed in terms of HFB1 concentrations in samples B and C (FIG. 6). A complete detoxification was observed at 40 U/kg.

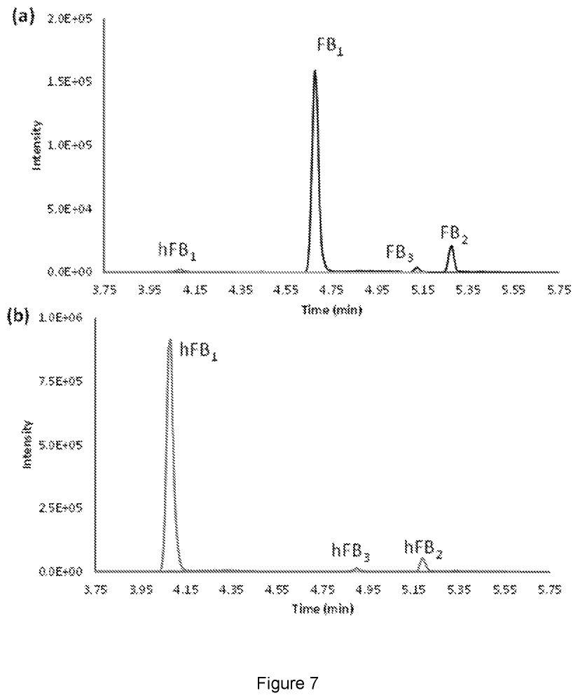

Other Fusarium mycotoxins were also detected in these samples including fusaric acid, FB2 and FB3. The levels of fusaric acid were consistent across samples A, B and C. However, FB2 and FB3 showed a similar pattern to what was observed for FB1; they were detected in A sample, and reduced in samples B and C. The reduction of FB2 and FB3, coincided with the appearance of HFB2 and HFB3, providing evidence that the esterase is removing the tricarballylic side chains of all fumonisins present (FIG. 7).

### Example 7: Recovery of a Polypeptide Having Fumonisin Esterase (FE) Activity Derived from Residual Yeast Biomass

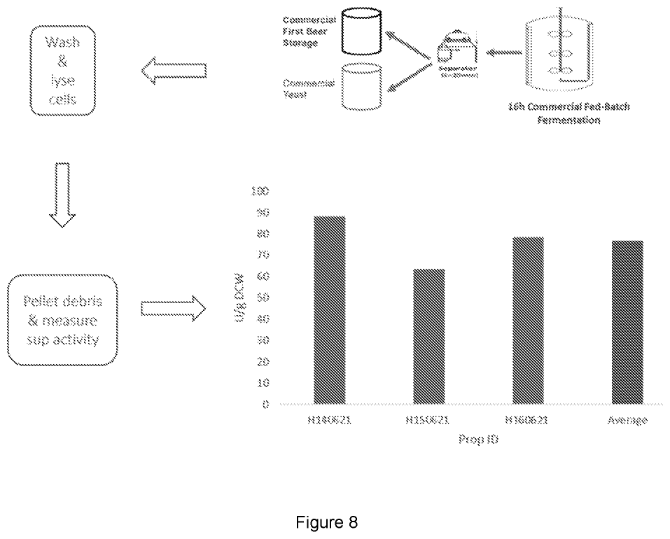

Strain M27750 (a strain of S. cerevisiae engineered to secrete the polypeptide having the amino acid sequence of SEQ ID NO: 14) was aerobically propagated at 20 L scale using a glucose-based media in a fed-batch process. Following completion of the propagation, the yeast biomass (commercial yeast) was physically separated from the spent media (commercial first beer storage). A sample of the yeast biomass was washed twice by diluting in sterile water, pelleting the cells by centrifugation, and removing the water supernatant. A portion of the washed cells were used to determine dry cell weight using a TGM800 moisture analyzer. A separate aliquot of the washed cells was resuspended in 20 mM Tris H 8, 0.1 mg/ml BSA and added to bead beater vial followed by mechanical cell lysis by bead beating for 20 seconds. The cell/beads were pelleted by centrifugation, and the supernatant removed to a new tube and spun again to remove any residual cell debris. The resulting supernatant was diluted 500 to 1000-fold and used in a TCA assay; the fumonisin esterase activity per gram dry cell weight was determined for three different propagations (H140621, H150621, and H160621), FIG. 8.

### Example 8: Use of a Polypeptide Having Fumonisin Esterase (FE) Activity During a Corn Mash Fermentation

The two strains M2390 (wild-type, non-genetically modified biofuel S. cerevisiae yeast) and M23192 (engineered to secrete the polypeptide having the amino acid sequence of SEQ ID NO: 14) where grown overnight in 55 mL YPD (10 g/L yeast extract, 20 g/L peptone, and 40 g/L dextrose) and a 250 mL baffled flask at 32° C. and 200 rpm overnight. The day of the experiment, 50 mL of the overnight cultures was spun at 4000 rpm for 3 minutes in a 50 ml falcon tube. Each strain's supernatant was decanted into a new sterile 50 ml conical tube. To measure the dry cell weight (DCW), 3 mL of each strain's supernatant was added back to the cell pellets and resuspended and measure on a Sautorius LMA200PM. A set of 10 g/L DCW total inoculums were then made up with several blended mixes of 100, 99, 98, 95, and 90% M2390 and 0, 1, 2, 5, 10% M23192 respectively with M2390's supernatant used to dilute. Industrial corn mash samples dosed with penicillin and Lactrol™ and were stored in 1 gallon aliquots at −10° C. until the day of the fermentation. The required frozen mash sample was removed from the freezer and thawed in a 60° C. water bath before being massed out into 6 mL serum vials for a final solids concentration of 30% total solids (TS). Ten (10) mg of fumonisin B1 (FB1) was dissolved into 1 ml ethanol for a 10,000-ppm stock solution. This stock solution was then added to the sterile makeup water for the fermentation at a final concentration of 25 ppm at the start of fermentation along with 0 ppm (added) controls. Yeast-produced FE3_S01D3 was concentrated from the supernatant of an aerobic propagation and spray dried in a matrix of 20% solids maltodextrin. For the single fermentation with the spray dried enzyme, 3 mg of the spray-dried powder was directed added to the serum vial before mash addition and M2390 inoculum.

Each condition was inoculated at a concentration of 0.3 g yeast/kg corn mash, exogenous glucoamylase enzyme loading of 0.45 AGU/g TS, sealed with crimped gray butyl rubber stoppers, and vented with 23-gauge needles. The vials were then incubated at 33° C. and 170 RPM for 24 hours and then decreased to 31° C. for the remainder of the fermentation. CO₂ production was monitored via ACAN (automated CO₂ analysis), and each condition was sampled for sugars, glycerol, and ethanol by HPLC with fumonisin and derivatives quantified by mass spectrometry at hour 66.

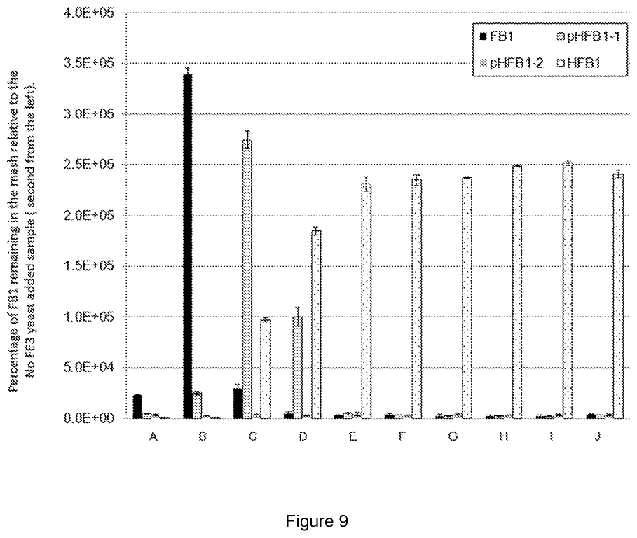

All conditions showed similar CO₂ production, residual sugar, glycerol, and ethanol (data not shown). The addition of 1% of FE3 yeast M23192 decreased the FB1 by >90%, and the addition of 5% FE3 yeast converted >99% of the FB1 to pHFB1 or HFB1. The addition of the spray dried FE3 was also able to convert >99% of FB1 to pHFB1 or HFB1 (FIG. 9).

### Example 9: Inclusion of Polypeptide Having Fumonisin Esterase (FE) Activity in Animal Feed Pellets

A spray-dried preparation of an isolated polypeptide having fumonisin esterase activity (herein referred to as the “enzyme”) having the amino acid sequence of SEQ ID NO: 14 was combined with pig meal (Euronutrition) and used to produce feed pellets according to standard pelleting process. Briefly, the enzyme was incorporated in the meal at the site inclusion rate using a 1 kg laboratory blender (100 rpm). The pelletization was made using an internal Kahl press (Amandus Kahl press pelletizer model 14-175) equipped with a steam conditioner (650 rpm, 1.6 bar, steam valve opening depending on temperature target) allowing steam injection and temperature control.

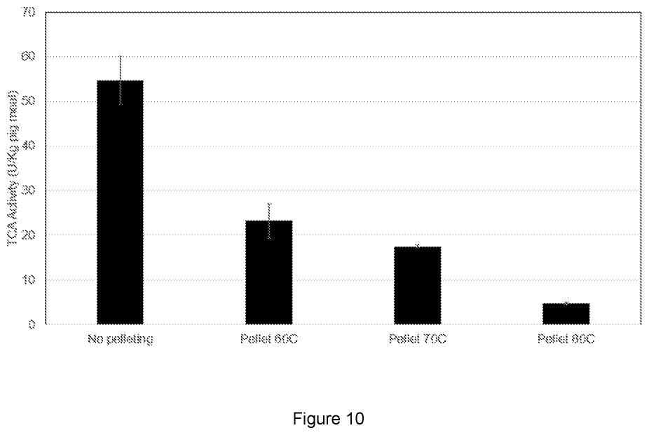

The feed pelleting was performed at three different temperatures 60, 70 and 80° C. A 10 g aliquot of each pellet sample was ground to a fine powder in separate mortar and pestles. From this ground material, triplicate 0.5 g samples were mixed with 1 ml buffer (20 mM Tris/HCL at pH8 with 0.1 mg/ml BSA). Samples were vigorously mixed and incubated at room temperature for 30 minutes. Samples were then spun at 6000 g for 4 minutes. Ten (10) ul of supernatant was added to 90 ul buffer containing 80 ppm fumonisin B1, incubated at 30° C. for 10 minutes followed by 99° C. for 15 minutes. Samples were run over HPLC filter columns and analyzed for TCA by LCMS. The TCA activity was also measured using the same method for a sample of the pig meal with an isolated polypeptide having fumonisin esterase activity that was removed prior to the pelleting process (no pelleting control). As shown on FIG. 10, the samples pelleted at 60, 70 and 80° C. retained 23.25, 17.5, and 4.75 U/kg TCA activity, respectively, compared to the “No pelleting” control at 54.7 U/kg.

### Example 10: Heterologous Expression of a Polypeptide Having a Fumonisin Esterase (FE) Activity in a Recombinant Bacteria Host Cell

The polypeptide having a FE activity having the amino acid sequence of SEQ ID NO: 40 was expressed from a plasmid (pMU4616) in a Lacticaseibacillus casei host strain. Table 7 provides the descriptions of the L. casei strains tested in the Example herein.

Table 7. Description of the Lacticaseibacillus casei strains tested in the present example.

| Strains | Description |
|---|---|
| M17745 | Parental Lacticaseibacillus casei bacteria (not used in |
| | experiment) |
| T4616 | M17745 containing plasmid pMU4616 (for the expression of the |
| | amino acid sequences of SEQ ID NO: 41 (signal sequence) and |
| | 40 (FE3 variants), the mature form of the polypeptide having |
| | the amino acid sequence of SEQ ID NO: 40). |
| T4567 | M17745 containing control plasmid pMU4567 (no polypeptide |
| | having fumonisin esterase is being expressed) |

Strains T4616 (N=4) and T4567 (N=2) were cultured in MRS culture media containing erythromycin (Erm) and 75 ppm fumonisin B1 (FB1) for 72 hours at 37° C. The resulting supernatants are the “culture” samples. The same strains were cultured in MRS culture media containing Erm for 24 hours at 37° C. The supernatants from this second set of cultures were incubated with 75 ppm FB1 for 72 hours at 37° C. (the “sup only” samples). The Erm was the selection to maintain the plasmids in the bacteria.

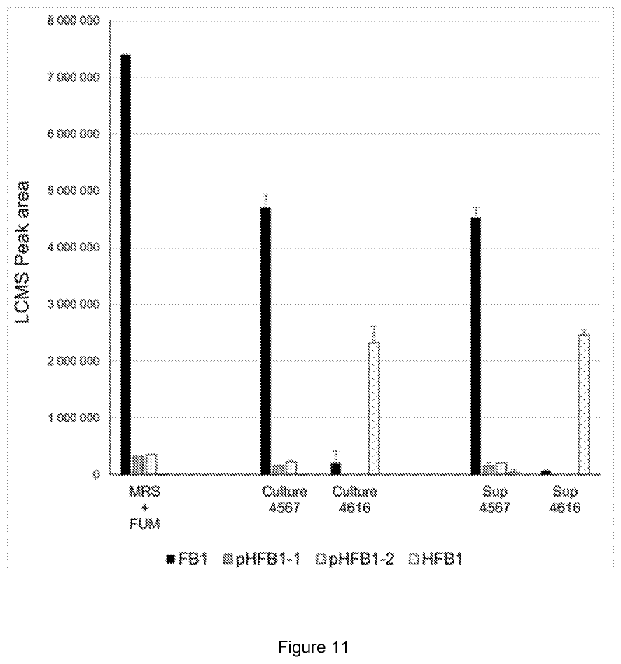

LCMS analysis of FB1 and its derivatives (i.e., pHFB1-1, pHFB1-2 and HFB1) was performed on both the “culture” and “sup only” samples. The T4616 strain exhibited secreted fumonisin esterase activity, as determined by LCMS (FIG. 11). Similar levels of conversion of FB1 to HFB1 were seen for the “culture” and “sup only” samples. The negative control strain (i.e., T4567) did not exhibit any fumonisin esterase activity in the “culture” or “sup only” samples.

### Example 11: In Vivo Efficacy Assessment of a Polypeptide Having a FE Activity when Supplemented in Mycotoxin Contaminated Feed for Post-Weaning Piglets

The effect of two different doses of a polypeptide having a FE activity (e.g., having the amino acid sequence of SEQ ID NO: 14) on the typical markers of fumonisin exposure were evaluated in piglets (28 d of age, 1 week after weaning) as model. Artificially fumonisin (FUM) contaminated feed (5 mg/kg feed) using contaminated corn meal was mixed with 2 different doses of polypeptide having a FE activity, namely 10 U/kg (Dose 1) and 20 U/kg feed (Dose 2), both fed at 2.5 g/kg. In addition, a control group (UUC) was fed with FUM free feed and no polypeptide having a FE activity and a positive control group (IUC) was fed with FUM contaminated feed and no polypeptide having a FE activity. A summary of the different treatment groups is provided in Table 8.

Table 8. Description of the different treatment groups

| | | Polypeptide | | | |
|---|---|---|---|---|---|
| | FUM | having FE | Animals | | Total |
| Group | (mg/kg | activity | per | | animals/ |
| name | feed) | (U/kg feed) | Replicate | Replicates | group |
| UUC | — | — | 2 | 5 | 10 |
| IUC | 5 | — | 2 | 5 | 10 |
| Dose 1 | 5 | 10 | 2 | 5 | 10 |
| Dose 2 | 5 | 20 | 2 | 5 | 10 |

The efficacy was investigated by evaluating general parameters such as the determination of the sphinganine:sphingosine ratio (Sa/So) in serum and the measurement of FUM and its derivatives (HFB1, pHFB1-1a, pHFB1-2b) in faeces.

Sphinganine (Sa) to sphingosine (So) ratio (Sa/So) are sensitive biomarkers of fumonisin B1 (FB1) exposure in animals. They correlate with liver and kidney toxicity and often precede signs of toxicity. In order to perform the Sa/So analysis, blood was collected from each piglet on days 14 and 20 (D14 and D20, respectively). The ratio of Sa/So in serum was determined by LC-MS/MS and the results are provided in Table 9. On both study days, D14 and D20, a significant lower Sa/So ratio was observed for both doses compared to IUC.

Table 9. Overview of the mean Sa/So in blood, standard deviation (SD), significant differences (P<0.05) of the groups compared to the IUC as reference (Pairwise P-value) and differences between all groups (Multiple P-Value).

| | Sa/So | Sa/So | Pairwise | Multiple |
|---|---|---|---|---|
| Groups | Mean | SD | P-value | P-value |
| IUC D 14 | 0.543 | 0.186 | REF | b |
| UUC D 14 | 0.132 | 0.015 | <0.001 | a |
| Dose 1 D 14 | 0.256 | 0.193 | <0.001 | a |
| Dose 2 D 14 | 0.196 | 0.049 | <0.001 | a |
| IUC D 20 | 0.866 | 0.264 | REF | b |
| UUC D 20 | 0.157 | 0.028 | <0.001 | a |
| Dose 1 D 20 | 0.335 | 0.160 | <0.001 | a |
| Dose 2 D 20 | 0.292 | 0.136 | <0.001 | a |

The concentrations of Fumonisins and their hydrolyzed metabolites (i.e., FB1, HFB1, FB2 and FB3) were measured by HPLC vs standards in faeces (about 10 g) collected on day 14 and 20 (D14 and D20, respectively) from each piglet. The results of theses analyses are provided in Table 10. Briefly, a significant lower concentration of FB1, FB2 and FB3 was detected for both treatment groups (dose 1 and dose 2) at both time points, except for FB1, for which a close to significance (P=0.068) lower concentration was found at D20 for dose 1 compared to the IUC.

Table 10. Overview of the mean concentration (μg/kg) FB1, HFB1, FB2 and FB3 in faeces and standard deviation (SD), significant differences (P<0.05) of the groups compared to the IUC as reference (Pairwise P-value) and differences between all groups (Multiple P-Value)

| | | | | | Pair- | |
|---|---|---|---|---|---|---|
| | | Group | | | wise | Multiple |
| Fumonisin | Days | name | Mean | SD | P-value | P-value |
| FB1 | D 14 | IUC | 3627.230 | 1926.789 | REF | b |
| | | UUC | 9.163 | 4.619 | <0.001 | a |
| | | Dose 1 | 1200.624 | 939.824 | 0.002 | a |
| | | Dose 2 | 991.288 | 559.244 | 0.001 | a |
| | D 20 | IUC | 1313.621 | 959.987 | REF | b |
| | | UUC | 144.405 | 424.990 | 0.001 | a |
| | | Dose 1 | 745.655 | 590.718 | 0.068 | ab |
| | | Dose 2 | 591.480 | 330.882 | 0.024 | ab |
| HFB1 | D 14 | IUC | 99.147 | 106.660 | REF | a |
| | | UUC | 14.436 | 34.148 | 0.73 | a |
| | | Dose 1 | 1449.883 | 795.709 | <0.001 | b |
| | | Dose 2 | 1870.570 | 409.889 | <0.001 | b |
| | D 20 | IUC | 223.093 | 290.062 | REF | a |
| | | UUC | 38.448 | 94.847 | 0.486 | a |
| | | Dose 1 | 1446.293 | 718.899 | <0.001 | b |
| | | Dose 2 | 1801.212 | 531.090 | <0.001 | b |
| FB2 | D 14 | IUC | 2266.177 | 587.603 | REF | c |
| | | UUC | 18.303 | 20.380 | <0.001 | a |
| | | Dose 1 | 981.718 | 381.159 | <0.001 | b |
| | | Dose 2 | 627.871 | 258.245 | <0.001 | b |
| | D 20 | IUC | 1255.979 | 578.411 | REF | c |
| | | UUC | 81.632 | 206.289 | <0.001 | a |
| | | Dose 1 | 702.346 | 227.909 | 0.002 | b |
| | | Dose 2 | 447.876 | 168.783 | <0.001 | ab |
| FB3 | D 14 | IUC | 384.298 | 133.031 | REF | c |
| | | UUC | 2.875 | 2.048 | <0.001 | a |
| | | Dose 1 | 148.471 | 80.402 | <0.001 | b |
| | | Dose 2 | 103.263 | 54.024 | <0.001 | ab |
| | D 20 | IUC | 173.609 | 92.894 | REF | c |
| | | UUC | 12.626 | 33.523 | <0.001 | a |
| | | Dose 1 | 90.608 | 41.400 | 0.004 | b |
| | | Dose 2 | 67.183 | 27.899 | 0.001 | ab |

While the invention has been described in connection with specific embodiments thereof, it will be understood that the scope of the claims should not be limited by the preferred embodiments set forth in the examples, but should be given the broadest interpretation consistent with the description as a whole.

1. A polypeptide having fumonisin esterase activity having at least 70% identity with the amino acid sequence of SEQ ID NO: 1, and
wherein the amino residues at positions 487, 488, 489 and 490 are independently present or absent; and
wherein at least one of:
the amino acid residue at position 11 is different from a valine residue;
the amino acid residue at position 19 is different from a methionine residue;
the amino acid residue at position 22 is different from an arginine residue;
the amino acid residue at position 33 is different from a glycine residue;
the amino acid residue at position 41 is different from a histidine residue;
the amino acid residue at position 87 is different from a glycine residue;
the amino acid residue at position 129 is different from a phenylalanine residue;
the amino acid residue at position 136 is different from a leucine residue;
the amino acid residue at position 173 is different from an arginine residue;
the amino acid residue at position 265 is different from an arginine residue;
the amino acid residue at position 269 is different from a threonine residue;
the amino acid residue at position 284 is different from an alanine residue;
the amino acid residue at position 287 is different from an alanine residue;
the amino acid residue at position 295 is different from an arginine residue;
the amino acid residue at position 313 is different from a proline residue;
the amino acid residue at position 314 is different from a methionine residue;
the amino acid residue at position 327 is different from a glutamine residue;
the amino acid residue at position 374 is different from a glutamine residue;
the amino acid residue at position 376 is different from an alanine residue;
the amino acid residue at position 384 is different from an asparagine residue;
the amino acid residue at position 389 is different from a glycine residue;
the amino acid residue at position 424 is different from a proline residue;
the amino acid residue at position 430 is different from a glycine residue;
the amino acid residue at position 468 is different from a glutamic acid residue;
the amino acid residue at position 482 is different from a proline residue; or
the amino acid residue at position 487, when present, is different from an alanine residue; and
wherein the polypeptide has increased residual fumonisin esterase activity, when measured after a heat challenge at a temperature of at least 37° C. for at least 1 minute, when compared to the control polypeptide comprising the amino acid of SEQ ID NO: 3.

2. The polypeptide of claim 1, wherein at least one of:
the amino acid residue at position 11 comprises a hydrophobic side chain;
the amino acid residue at position 19 comprises a hydrophobic side chain;
the amino acid residue at position 22 comprises a positively-charged side chain;
the amino acid residue at position 33 comprises a negatively-charged side chain;
the amino acid residue at position 41 comprises a polar uncharged side chain;
the amino acid residue at position 87 comprises a negatively-charged side chain;
the amino acid residue at position 129 comprises a hydrophobic side chain;
the amino acid residue at position 136 comprises a hydrophobic side chain;
the amino acid residue at position 173 comprises a hydrophobic side chain;
the amino acid residue at position 265 comprises a positively-charged side chain;
the amino acid residue at position 269 comprises a hydrophobic side chain;
the amino acid residue at position 284 comprises a negatively-charged side chain;
the amino acid residue at position 287 comprises a hydrophobic side chain;
the amino acid residue at position 313 comprises a polar uncharged side chain;
the amino acid residue at position 314 comprises a hydrophobic side chain;
the amino acid residue at position 327 comprises a hydrophobic side chain;
the amino acid residue at position 374 comprises a positively-charged side chain;
the amino acid residue at position 376 comprises a hydrophobic side chain;
the amino acid residue at position 384 comprises a hydrophobic side chain;
the amino acid residue at position 389 comprises a negatively-charged side chain;
the amino acid residue at position 424 comprises a hydrophobic side chain;
the amino acid residue at position 468 comprises a positively-charged side chain;
the amino acid residue at position 482 comprises a polar uncharged side chain; or
the amino acid residue at position 487, when present, comprises a hydrophobic side chain.

3. The polypeptide of claim 2, wherein at least one of:
the amino acid residue at position 11 is a leucine residue;
the amino acid residue at position 19 is an isoleucine residue;
the amino acid residue at position 22 is a lysine residue;
the amino acid residue at position 33 is an aspartic acid residue;
the amino acid residue at position 41 is a glutamine residue;
the amino acid residue at position 87 is an aspartic acid residue;
the amino acid residue at position 129 is a tyrosine residue;
the amino acid residue at position 136 is a phenylalanine residue;
the amino acid residue at position 173 is an isoleucine residue;
the amino acid residue at position 265 is a lysine residue;
the amino acid residue at position 269 is a leucine residue;
the amino acid residue at position 284 is an aspartic acid residue;
the amino acid residue at position 287 is a valine residue;
the amino acid residue at position 295 is a proline residue;
the amino acid residue at position 313 is a serine residue;
the amino acid residue at position 314 is a leucine or an isoleucine residue;
the amino acid residue at position 327 is a leucine residue;
the amino acid residue at position 374 is a histidine residue;
the amino acid residue at position 376 is a valine residue;
the amino acid residue at position 384 is an isoleucine residue;
the amino acid residue at position 389 is an aspartic acid residue;
the amino acid residue at position 424 is a leucine residue;
the amino acid residue at position 430 is a cysteine residue;
the amino acid residue at position 468 is a lysine residue;
the amino acid residue at position 482 is a serine residue; or
the amino acid residue at position 487, when present, is a valine residue.

4. (canceled)

5. The polypeptide of claim 1 having the amino acid sequence of one of SEQ ID NO: 6 to 32 or 40.

6. The polypeptide of claim 1, wherein the polypeptide comprises the amino acid sequence of SEQ ID NO: 2,
wherein the amino residues at positions 487, 488, 489 and 490 are independently present or absent; and
wherein at least one of:
the amino acid residue at position 19 is different from a methionine residue;
the amino acid residue at position 33 is different from a glycine residue;
the amino acid residue at position 87 is different from a glycine residue;
the amino acid residue at position 136 is different from a leucine residue;
the amino acid residue at position 265 is different from an arginine residue;
the amino acid residue at position 269 is different from a threonine residue;
the amino acid residue at position 284 is different from an alanine residue;
the amino acid residue at position 295 is different from an arginine residue;
the amino acid residue at position 314 is different from a methionine residue;
the amino acid residue at position 327 is different from a glutamine residue;
the amino acid residue at position 374 is different from a glutamine residue;
the amino acid residue at position 376 is different from an alanine residue;
the amino acid residue at position 384 is different from an asparagine residue;
the amino acid residue at position 389 is different from a glycine residue;
the amino acid residue at position 430 is different from a glycine residue;
the amino acid residue at position 482 is different from a proline residue; or
the amino acid residue at position 487, when present is different from an alanine residue.

7. The polypeptide of claim 6, wherein at least one of:
the amino acid residue at position 19 comprises a hydrophobic side chain;
the amino acid residue at position 33 comprises a negatively-charged side chain;
the amino acid residue at position 87 comprises a negatively-charged side chain;
the amino acid residue at position 136 comprises a hydrophobic side chain;
the amino acid residue at position 265 comprises a positively-charged side chain;
the amino acid residue at position 269 comprises a hydrophobic side chain;
the amino acid residue at position 284 comprises a negatively-charged side chain;
the amino acid residue at position 314 comprises a hydrophobic side chain;
the amino acid residue at position 327 comprises a hydrophobic side chain;
the amino acid residue at position 374 comprises a positively-charged side chain;
the amino acid residue at position 376 comprises a hydrophobic side chain;
the amino acid residue at position 384 comprises a hydrophobic side chain;
the amino acid residue at position 389 comprises a negatively-charged side chain;
the amino acid residue at position 482 comprises a polar uncharged side chain; or
the amino acid residue at position 487, when present, comprises a hydrophobic side chain.

8. The polypeptide of claim 7, wherein at least one of:
the amino acid residue at position 19 is an isoleucine residue;
the amino acid residue at position 33 is an aspartic acid residue;
the amino acid residue at position 87 is an aspartic acid residue;
the amino acid residue at position 136 is a phenylalanine residue;
the amino acid residue at position 265 is a lysine residue;
the amino acid residue at position 269 is a leucine residue;
the amino acid residue at position 284 is an aspartic acid residue;
the amino acid residue at position 295 is a proline residue;
the amino acid residue at position 314 is a leucine or an isoleucine residue;
the amino acid residue at position 327 is a leucine residue;
the amino acid residue at position 374 is a histidine residue;
the amino acid residue at position 376 is a valine residue;
the amino acid residue at position 384 is an isoleucine residue;
the amino acid residue at position 389 is an aspartic acid residue;
the amino acid residue at position 430 is a cysteine residue;
the amino acid residue at position 482 is a serine residue; or
the amino acid residue at position 487, when present, is a valine residue.

9. (canceled)

10. The polypeptide of claim 1, wherein the polypeptide comprises the amino acid sequence of SEQ ID NO: 33 and:
the amino acid residue at position 19 is a methionine or an isoleucine residue;
the amino acid residue at position 22 is a lysine or an arginine residue;
the amino acid residue at position 33 is a glycine or a glutamic acid residue;
the amino acid residue at position 173 is an arginine or an isoleucine residue;
the amino acid residue at position 284 is an alanine or an aspartic acid residue;
the amino acid residue at position 313 is a proline or a serine residue;
the amino acid residue at position 468 is a glutamic acid or a lysine residue; and
the amino acid residue at position 482 is a proline or a serine residue.

11. The polypeptide of claim 6 having the one of the amino acid sequences of SEQ ID NO: 11, 12, 13 or 14.

12. (canceled)

13. (canceled)

14. A recombinant microbial host cell comprising a heterologous nucleic acid molecule encoding the polypeptide having fumonisin esterase activity of claim 1.

15.-23. (canceled)

24. A method of detoxifying a fumonisin mycotoxin, the method comprising contacting the polypeptide having fumonisin esterase activity of claim 1 with the fumonisin mycotoxin so as to cause the hydrolysis of at least one tricarballylic acid moiety from the fumonisin mycotoxin.

25.-33. (canceled)

34. A process for making a composition comprising the polypeptide having fumonisin esterase activity of claim 1, the process comprising:
a) propagating a recombinant microbial host cell comprising a heterologous nucleic acid molecule encoding the polypeptide having fumonisin esterase activity to obtain a propagated recombinant microbial host cell and the polypeptide having fumonisin esterase activity, the variant or the fragment;
b) optionally:
separating the propagated microbial host cell from the polypeptide having fumonisin esterase activity from at least one component of the propagated recombinant microbial host cell to obtain a separated fraction enriched in the polypeptide having fumonisin esterase activity;
inactivating the propagated microbial host cell to obtain an inactivated fraction;
lysing the propagated microbial host cell to obtain a lysed fraction;
drying, at least in part, the propagated microbial host cell, the separated fraction, the inactivated fraction or the lysed fraction to obtain a dried fraction;
c) optionally substantially purifying the polypeptide having fumonisin esterase activity from the separated fraction, the inactivated fraction, the lysed fraction or the dried fraction to obtain a purified fraction; and
d) formulating the propagated recombinant microbial host cell, the optionally separated fraction, the optionally inactivated fraction, the optionally lysed fraction, the optionally dried fraction, or the optionally substantially purified fraction into the composition.

35.-38. (canceled)
